(19) United States
(12) Patent Application Publication
(10) Pub. No.: US 2019/0345483 A1
(43) Pub. Date: Nov. 14, 2019

(54) AAV Split Cas9 Genome Editing and Transcriptional Regulation

(71) Applicant: President and Fellows of Harvard College, Cambridge, MA (US)

(72) Inventors: George M. Church, Brookline, MA (US); Wei Leong Chew, Boston, MA (US)

(21) Appl. No.: 16/099,828

(22) PCT Filed: May 12, 2017
(86) PCT No.: PCT/US17/32362

§ 371 Date: Nov. 8, 2018

Related U.S. Application Data
(60) Provisional application No. 62/335,271, filed on May 12, 2016.

(51) Int. Cl. C12N 15/10; C12N 15/86; C12N 15/90; C12N 9/22

(52) U.S. Cl. CPC C12N 15/1024; C12N 15/86; C12N 2310/20; C12N 9/22; C12N 15/90

(57) ABSTRACT

The invention provides methods of altering a target nucleic acid in a cell using the AAV split Cas9 platform. The methods comprise providing the cell an enzymatically active Cas9 and optionally a transcriptional regulator fused thereto and guide RNA having different spacer sequence lengths wherein the guide RNA directs the enzymatically active Cas9 and optionally a transcriptional regulator fused thereto to either cleave a target nucleic acid or regulate expression of a target nucleic acid.

### RELATED APPLICATION DATA

This application claims priority to U.S. Provisional Application No. 62/335,271 filed on May 12, 2016, which is hereby incorporated by reference in its entirety for all purposes.

### STATEMENT OF GOVERNMENT INTERESTS

This invention was made with government support under Grant No. P50 HG005550 awarded by National Institutes of Health. The government has certain rights in the invention.

### BACKGROUND

The CRISPR type II system is a recent development that has been efficiently utilized in a broad spectrum of species. See Friedland, A. E., et al., Heritable genome editing in C. elegans via a CRISPR-Cas9 system. Nat Methods, 2013. 10(8): p. 741-3, Mali, P., et al., RNA-guided human genome engineering via Cas9. Science, 2013. 339(6121): p. 823-6, Hwang, W X., et al., Efficient genome editing in zebrafish using a CRISPR-Cas system. Nat Biotechnol, 2013, Jiang, W., et al., RNA-guided editing of bacterial genomes using CRISPR-Cas systems. Nat Biotechnol, 2013, Jinek, M., et al., RNA-programmed genome editing in human cells. elife, 2013. 2: p. e00471, Cong, L., et al., Multiplex genome engineering using CRISPR/Cas systems. Science, 2013. 339(6121): p. 819-23, Yin, H., et al., Genome editing with Cas9 in adult mice corrects a disease mutation and phenotype. Nat Biotechnol, 2014. 32(6): p. 551-3. CRISPR is particularly customizable because the active form consists of an invariant Cas9 protein and an easily programmable guide RNA (gRNA). See Jinek, M., et al., A programmable dual-RNA-guided DNA endonuclease in adaptive bacterial immunity. Science, 2012. 337(6096): p. 816-21. Of the various CRISPR orthologs, the Streptococcus pyogenes (Sp) CRISPR is the most well-characterized and widely used. The Cas9-gRNA complex first probes DNA for the protospacer-adjacent motif (PAM) sequence (NGG for Sp Cas9), after which Watson-Crick base-pairing between the gRNA and target DNA proceeds in a ratchet mechanism to form an R-loop. Following formation of a ternary complex of Cas9, gRNA, and target DNA, the Cas9 protein generates two nicks in the target DNA, creating a blunt double-strand break (DSB) that is predominantly repaired by the non-homologous end joining (NHEJ) pathway or, to a lesser extent, template-directed homologous recombination (HR). CRISPR methods are disclosed in U.S. Pat. Nos. 9,023,649 and 8,697,359. See also, Fu et al., Nature Biotechnology, Vol. 32, Number 3, pp. 279-284 (2014). Additional references describing CRISPR-Cas9 systems including nuclease null variants (dCas9) and nuclease null variants functionalized with effector domains such as transcriptional activation domains or repression domains include J. D. Sander and J. K. Joung, Nature biotechnology 32 (4), 347 (2014); P. D. Hsu, E. S. Lander, and F. Zhang, Cell 157 (6), 1262 (2014); L. S. Qi, M. H. Larson, L. A. Gilbert et al., Cell 152 (5), 1173 (2013); P. Mali, J. Aach, P. B. Stranges et al., Nature biotechnology 31 (9), 833 (2013); M. L. Maeder, S. J. Linder, V. M. Cascio et al., Nature methods 10 (10), 977 (2013); P. Perez-Pinera, D. D. Kocak, C. M. Vockley et al., Nature methods 10 (10), 973 (2013); L. A. Gilbert, M. H. Larson, L. Morsut et al., Cell 154 (2), 442 (2013); P. Mali, K. M. Esvelt, and G. M. Church, Nature methods 10 (10), 957 (2013); and K. M. Esvelt, P. Mali, J. L. Braff et al., Nature methods 10 (11), 1116 (2013).

The CRISPR-Cas9 system enables facile genetic and epigenetic manipulations (Mali, P. et al. RNA-guided human genome engineering via Cas9. Science 339, 823-826, doi:10.1126/science.1232033 (2013); Cong, L. et al. Multiplex genome engineering using CRISPR/Cas systems. Science 339, 819-823, doi:10.1126/science.1231143 (2013); Jinek, M. et al. A programmable dual-RNA-guided DNA endonuclease in adaptive bacterial immunity. Science 337, 816-821, doi:10.1126/science.1225829 (2012)), and its simplicity and robustness suggest that personalized genetic therapeutics may be within reach. As CRISPR-Cas9 approaches the clinic, efficacy and safety for the patient would be of paramount importance. Extensive efforts have been employed to deliver CRISPR-Cas9 with adeno-associated viruses (AAVs). AAVs are prevalent and serologically compatible with a large fraction of the human population (Gao, G et al. Clades of Adeno-associated viruses are widely disseminated in human tissues. Journal of virology 78, 6381-6388, doi:10.1128/JVI.78.12.6381-6388.2004 (2004); Boutin, S. et al. Prevalence of serum IgG and neutralizing factors against adeno-associated virus (AAV) types 1, 2, 5, 6, 8, and 9 in the healthy population: implications for gene therapy using AAV vectors. Hum Gene Ther 21, 704-712, doi:10.1089/hum.2009.182 (2010)) and are generally not considered to be pathogenic. Furthermore, AAVs allow both programmable tissue-tropism and systemic delivery (Zincarelli, C., Soltys, S., Rengo, G & Rabinowitz, J. E. Analysis of AAV serotypes 1-9 mediated gene expression and tropism in mice after systemic injection. Mol Ther 16, 1073-1080, doi:10.1038/mt.2008.76 (2008)). The preclinical promise of AAV-CRISPR-Cas9 by correcting genetic defects in mice has been described (Ran, F. A. et al. In vivo genome editing using Staphylococcus aureus Cas9. Nature 520, 186-191, doi:10.1038/nature14299 (2015); Nelson, C. E. et al. In vivo genome editing improves muscle function in a mouse model of Duchenne muscular dystrophy. Science 351, 403-407, doi:10.1126/science.aad5143 (2016); Tabebordbar, M. et al. In vivo gene editing in dystrophic mouse muscle and muscle stem cells. Science 351, 407-411, doi:10.1126/science.aad5177 (2016); Long, C. et al. Postnatal genome editing partially restores dystrophin expression in a mouse model of muscular dystrophy. Science 351, 400-403, doi:10.1126/science.aad5725 (2016); Yang, Y. et al. A dual AAV system enables the Cas9-mediated correction of a metabolic liver disease in newborn mice. Nature biotechnology, doi:10.1038/nbt.3469 (2016); Yin, H. et al. Therapeutic genome editing by combined viral and non-viral delivery of CRISPR system components in vivo. Nature biotechnology, doi:10.1038/nbt.3471 (2016)).

Further application of AAV-CRISPR-Cas9 for modulating postnatal chromatin status or gene expression would vest profound biological control, particularly in treating diseases resulting from epigenetic alterations irresolvable by genome-editing. However, this ability has yet to be realized, in part because the large Cas9 transgenes leave little space for additional function-conferring elements within current designs (AAV payload limit ≤4.7 kb) (Ran, F. A. et al. In vivo genome editing using Staphylococcus aureus Cas9. Nature 520, 186-191, doi:10.1038/nature14299 (2015); Nelson, C. E. et al. In vivo genome editing improves muscle function in a mouse model of Duchenne muscular dystrophy. Science 351, 403-407, doi:10.1126/science.aad5143 (2016); Tabebordbar, M. et al. In vivo gene editing in dystrophic mouse muscle and muscle stem cells. Science 351, 407-411, doi:10.1126/science.aad5177 (2016); Long, C. et al. Postnatal genome editing partially restores dystrophin expression in a mouse model of muscular dystrophy. Science 351, 400-403, doi:10.1126/science.aad5725 (2016); Yang, Y. et al. A dual AAV system enables the Cas9-mediated correction of a metabolic liver disease in newborn mice. Nature biotechnology, doi:10.1038/nbt.3469 (2016); Yin, H. et al. Therapeutic genome editing by combined viral and non-viral delivery of CRISPR system components in vivo. Nature biotechnology, doi:10.1038/nbt.3471 (2016); Senis, E. et al. CRISPR/Cas9-mediated genome engineering: an adeno-associated viral (AAV) vector toolbox. Biotechnology journal 9, 1402-1412, doi:10.1002/biot.201400046 (2014); Swiech, L. et al. In vivo interrogation of gene function in the mammalian brain using CRISPR-Cas9. Nature biotechnology 33, 102-106, doi:10.1038/nbt.3055 (2015)). This obstacle is exacerbated with the most widely used, but larger, Streptococcus pyogenes Cas9 (SpCas9, 4.2 kb), which makes packaging of even the minimum functional cassette extremely challenging (Long, C. et al. Postnatal genome editing partially restores dystrophin expression in a mouse model of muscular dystrophy. Science 351, 400-403, doi:10.1126/science.aad5725 (2016); Senis, E. et al. CRISPR/Cas9-mediated genome engineering: an adeno-associated viral (AAV) vector toolbox. Biotechnology journal 9, 1402-1412, doi:10.1002/biot.201400046 (2014); Swiech, L. et al. In vivo interrogation of gene function in the mammalian brain using CRISPR-Cas9. Nature biotechnology 33, 102-106, doi:10.1038/nbt.3055 (2015)).

### SUMMARY

Aspects of the present disclosure are directed to the use of split Cas9 to perform CRISPR-based methods in cells. According to one aspect, two or more portions or segments of a Cas9 are provided to a cell, such as by being expressed from corresponding nucleic acids introduced into the cell. The two or more portions are combined within the cell to form the Cas9 which has an ability to colocalize with guide RNA at a target nucleic acid. It is to be understood that the Cas9 may have one or more modifications from a full length Cas9 known to those of skill in the art, yet still retain or have the capability of colocalizing with guide RNA at a target nucleic acid. Accordingly, the two or more portions or segments, when joined together, need only produce or result in a Cas9 which has an ability to colocalize with guide RNA at a target nucleic acid.

According to certain general aspects, when a foreign nucleic acid sequence or sequences are expressed by the cell, the two or more portions or segments of an RNA guided DNA binding protein, such as Cas9, are produced and joined together to produce the RNA guided DNA binding protein, such as Cas9. When a foreign nucleic acid sequence or sequences are expressed by the cell, one or more or a plurality of guide RNAs are produced. The RNA guided DNA binding protein, such as Cas9, and a guide RNA produces a complex of the RNA guided DNA binding protein, the guide RNA and a double stranded DNA target sequence. In this aspect, the RNA is said to guide the DNA binding protein to the double stranded DNA target sequence for binding thereto. This aspect of the present disclosure may be referred to as co-localization of the RNA and DNA binding protein to or with the double stranded DNA.

DNA binding proteins within the scope of the present disclosure may include those which create a double stranded break (which may be referred to as a DNA binding protein nuclease), those which create a single stranded break (referred to as a DNA binding protein nickase) or those which have no nuclease activity (referred to as a nuclease null DNA binding protein) but otherwise bind to target DNA. In this manner, a DNA binding protein-guide RNA complex may be used to create a double stranded break at a target DNA site, to create a single stranded break at a target DNA site or to localize a transcriptional regulator or functional group, function-conferring protein or domain, which may be expressed by the cell, at a target DNA site so as to regulate expression of target DNA. According to certain aspects, the foreign nucleic acid sequence may encode one or more of a DNA binding protein nuclease, a DNA binding protein nickase or a nuclease null DNA binding protein. The foreign nucleic acid sequence may also encode one or more transcriptional regulator or functional group, function-conferring proteins or domains or one or more donor nucleic acid sequences that are intended to be inserted into the genomic DNA. According to one aspect, the foreign nucleic acid sequence encoding an RNA guided enzymatically active DNA binding protein further encodes the transcriptional regulator or functional group, function-conferring protein or domain fused to the RNA guided enzymatically active DNA binding protein.

Accordingly, expression of a foreign nucleic acid sequence by a cell may result in a double stranded break, a single stranded break and/or transcriptional activation or repression of the genomic DNA. Donor DNA may be inserted at the break site by cell mechanisms such as homologous recombination or nonhomologous end joining. It is to be understood that expression of a foreign nucleic acid sequence as described herein may result in a plurality of double stranded breaks or single stranded breaks at various locations along target genomic DNA, including one or more or a plurality of gene sequences, as desired.

Aspects of the present disclosure are directed to methods of using an enzymatically active Cas9, such as a Cas9 nuclease or nickase, optionally having a functional group attached thereto, and a guide RNA which is used to guide the enzymatically active Cas9 with the functional group attached thereto to a target nucleic acid. According to one aspect, when a functional group is attached to the enzymatically active Cas9, the functional group is directed to a target nucleic acid to perform the desired function on the target nucleic acid, such as transcriptional regulation. Also, it is to be understood that transcriptional regulation can also be accomplished according to methods described herein where an enzymatically active Cas9 is used without any attached functional group and transcriptional regulation is accomplished by inhibition of transcription due to the Cas9 forming a complex at the target nucleic acid and without cutting the target nucleic acid. According to one aspect, the guide RNA includes a truncated spacer sequence having a length sufficient to bind to a target nucleic acid and form a complex with the enzymatically active Cas9 optionally with the functional group attached thereto, but insufficient for the enzymatically active Cas9 to function to cut or nick the target nucleic acid. Without wishing to be bound by scientific theory, based on the length of the spacer sequence of the guide RNA, the endonucleolytic activity of the enzymatically active Cas9 is blocked or prevented or otherwise inhibited, and the otherwise enzymatically active Cas9 is effectively rendered a nuclease null Cas9. According to this aspect, the functional group when attached to the enzymatically active Cas9 performs the desired function of the functional group, as the enzymatically active Cas9 nuclease does not function to cut or nick the target nucleic acid. Without wishing to be bound by any scientific theory, the enzymatically active Cas9 optionally with the functional group attached thereto forms a co-localization complex with the guide RNA and the target nucleic acid, however, the length of the truncated spacer sequence of the guide RNA results in an inability of the Cas9 to cleave the target nucleic acid substrate.

According to another aspect, the present disclosure are directed to methods of using a enzymatically active Cas9, such as a Cas9 nuclease or nickase, optionally having a functional group attached thereto and a guide RNA with a spacer sequence having a length sufficient to bind to a target nucleic acid and to form a complex with the enzymatically active Cas9 optionally with the functional group attached thereto, and sufficient to allow the enzymatically active Cas9 to function as a nuclease or nickase with respect to the target nucleic acid. According to one aspect, the functional group when optionally attached to the enzymatically active Cas9 does not perform the desired function of the functional group, as the target nucleic acid is either cut or nicked by the enzymatically active Cas9.

Aspects of the present disclosure are directed to methods of using a enzymatically active Cas9, such as a Cas9 nuclease or nickase, optionally having a functional group attached thereto, a first guide RNA with a spacer sequence having a length sufficient to bind to a first target nucleic acid and form a complex with the enzymatically active Cas9 optionally having the functional group attached thereto, but insufficient to allow the enzymatically active Cas9 to function as a nuclease or nickase with respect to the first target nucleic acid, and a second guide RNA with a spacer sequence having a length sufficient to bind to a second target nucleic acid and form a complex with the enzymatically active Cas9 optionally having the functional group attached thereto, such as a Cas9 nuclease or nickase, and sufficient to allow the enzymatically active Cas9 to function as a nuclease or nickase with respect to the second target nucleic acid. According to this aspect, the enzymatically active Cas9 when complexed with the first guide RNA at the first target nucleic acid will function as a nuclease null Cas9 to deliver a functional group if present to the first target nucleic acid and the enzymatically active Cas9 when complexed with the second guide RNA at the second target nucleic acid will also function as a nuclease or nickase to either cut or nick the second target nucleic acid.

Aspects of the present disclosure are directed to programmable genome editing as an enzymatically active Cas9 can be used to cut or nick a target nucleic acid by selection of a first guide RNA sequence and the same Cas9 can be effectively rendered nuclease null by selection of a second guide RNA sequence which allows the Cas9 to complex at the target nucleic acid sequence but not cut or nick the target nucleic acid sequence. Such complex formation can have an inhibitory effect on transcription and therefore can regulate gene expression without using a separate transcription regulator functional group.

Aspects of the present disclosure are directed to programmable genome editing and use of a functional group, such as a transcriptional regulator, using the same species of enzymatically active Cas9 having the functional group attached thereto. Methods described herein are directed to the use of a single species of enzymatically active Cas9 having a transcriptional regulator attached thereto which can be simultaneously used for genome editing of target nucleic acids and transcriptional regulation of genes, based on the spacer sequence length of the particular guide RNA. The length of the guide RNA spacer sequence determines the ability of the enzymatically active Cas9 species having a functional group (such as a transcriptional regulator) attached thereto to either (1) function to deliver the functional group to a target nucleic acid so that the functional group can perform its desired function or (2) function as an enzyme to cut or nick a target nucleic acid.

According to certain aspects, the enzymatically active Cas9 optionally having a functional group attached thereto is present within a cell and two or more guide RNAs are provided to a cell in series or simultaneously wherein each guide RNA is designed to complex with the enzymatically active Cas9 optionally having a functional group attached thereto at respective target nucleic acid sites or sequences. Each guide RNA has a spacer sequence length that determines whether the enzymatically active Cas9 optionally having a functional group attached thereto will function as either an enzyme to cut or nick a nucleic acid or as a nuclease null Cas9 to form a complex at the target nucleic acid and deliver the functional group if present to a target nucleic acid so that the functional group may perform its function on a target nucleic acid. In this manner, enzymatically active Cas9 optionally having a functional group attached thereto may first be used to cut or nick a nucleic acid and then be used to deliver a functional group if present to a nucleic acid sequence so that the functional group may perform the function or vice versa. According to one aspect, a plurality of guide RNAs may be used to target the enzymatically active Cas9 optionally having a functional group attached thereto, such as a single species of an enzymatically active Cas9 optionally having a functional group attached thereto, to a plurality of different target nucleic acid sites to perform either cutting or nicking or functional group delivery.

When the enzymatically active Cas9 optionally having a functional group attached thereto is used for cutting or nicking a target nucleic acid, methods described herein contemplate the use of one or more donor nucleic acids that may be inserted into one or more cut or nick sites through homologous recombination or nonhomologous end joining. Accordingly, methods described herein are directed to methods of genome editing using the enzymatically active Cas9 optionally having a functional group attached thereto and also methods of targeting a functional group when present to a target nucleic acid to perform the function of the functional group using the enzymatically active Cas9 having a functional group attached thereto. One of skill will readily understand that the utility of the enzymatically active Cas9 optionally having a functional group attached thereto is determined by the spacer sequence length of the guide RNA and whether the guide RNA has a spacer sequence length that facilitates enzymatic activity of the enzymatically active Cas9 or not.

According to one aspect, a functional group may be any desired functional group as known to those of skill in the art. An exemplary functional group may be an effector domain, such as a transcriptional activator or transcriptional repressor, or a detectable group, such as fluorescent protein, or a binding functional group, such as an aptamer or a protein-protein binding domain, which can be used to bind to a desired functional group or a nuclear localization signal, which can be used to deliver the Cas9 to a nucleus.

According to certain aspects, a guide RNA that allows enzymatic activity of the enzymatically active Cas9 having a functional group attached thereto includes a spacer sequence having an exemplary nucleotide length of between about 25 and about 15 nucleotides, such as between about 20 and about 16 nucleotides. According to certain aspects, a guide RNA that inhibits enzymatic activity of the enzymatically active Cas9 optionally having a functional group attached thereto includes a spacer sequence having an exemplary nucleotide length of between about 8 and about 16 nucleotides. According to certain aspects, a guide RNA that inhibits enzymatic activity of the enzymatically active Cas9 optionally having a functional group attached thereto includes a spacer sequence having an exemplary nucleotide length of between 8, 9, 10, 11, 12, 13, 14, 15, 16, 17, 18, 19 and 20 nucleotides. A truncated spacer sequence has a nucleotide length that is shorter than the full length spacer sequence of the corresponding guide RNA.

According to certain aspects, a guide RNA includes a spacer sequence and a tracr mate sequence forming a crRNA, as is known in the art. According to certain aspects, a tracr sequence, as is known in the art, is also used in the practice of methods described herein. According to one aspect, the tracr sequence and the crRNA sequence may be separate or connected by the linker, as is known in the art.

According to one aspect, the present disclosure provides a method of altering a target nucleic acid in a cell including providing to the cell a first nucleic acid encoding a first portion of a Cas9 protein and a guide RNA (gRNA), providing to the cell a second nucleic acid encoding a second portion of the Cas9 protein and optionally a transcriptional regulator, wherein the cell expresses the first portion of the Cas9 protein, the gRNA and the second portion of the Cas9 protein or the second portion of the Cas9 and the transcriptional regulator fusion protein, and wherein the first portion of the Cas9 protein and the second portion of the Cas9 protein, or the first portion of the Cas9 protein and the second portion of the Cas9 and the transcriptional regulator fusion protein are joined together to form the Cas9 protein or the Cas9 fusion protein, wherein the gRNA and the Cas9 protein, or the gRNA and the Cas9 fusion protein form a co-localization complex with the target nucleic acid and alter the expression of the target nucleic acid.

According to another aspect, the present disclosure provides a method of altering a target nucleic acid in a cell of a subject including delivering to the cell of the subject a first nucleic acid encoding a first portion of a Cas9 protein and a guide RNA (gRNA) wherein the first nucleic acid is within a first vector, delivering to the cell of the subject a second nucleic acid encoding a second portion of the Cas9 protein and optionally a transcriptional regulator wherein the second nucleic acid is within a second vector, wherein the cell expresses the first portion of the Cas9 protein, the gRNA and the second portion of the Cas9 protein or the second portion of the Cas9 and the transcriptional regulator fusion protein, wherein the first portion of the Cas9 protein and the second portion of the Cas9 protein, or the first portion of the Cas9 protein and the second portion of the Cas9 and the transcriptional regulator fusion protein are joined together to form the Cas9 protein or the Cas9 fusion protein, and wherein the gRNA and the Cas9 protein, or the gRNA and the Cas9 fusion protein form a co-localization complex with the target nucleic acid and alter the expression of the target nucleic acid.

According to still another aspect, the present disclosure provides a method of modulating a target gene expression in a cell including providing to the cell a first recombinant adeno-associated virus comprising a first nucleic acid encoding an N-terminal portion of the Cas9 protein (Cas9^{N}) and a gRNA, providing to the cell a second recombinant adeno-associated virus comprising a second nucleic acid encoding a fusion protein comprising a C-terminal portion of the Cas9 protein (Cas9^{C}) fused with a transcriptional regulator (TR), wherein the cell expresses the Cas9N protein and the Cas9^{C}-TR fusion protein and join them to form a full length Cas9^{FL}-TR fusion protein, and wherein the cell expresses the gRNA, and the gRNA directs the Cas9^{FL}-TR fusion protein to the target gene and modulates target gene expression.

According to yet another aspect, the present disclosure provides a method of imaging a target nucleic acid in a cell including providing to the cell a first recombinant adeno-associated virus comprising a first nucleic acid encoding an N-terminal portion of the Cas9 protein (Cas9^{N}) and a gRNA, providing to the cell a second recombinant adeno-associated virus comprising a second nucleic acid encoding a fusion protein comprising a C-terminal portion of the Cas9 protein (Cas9^{C}) fused with a fluorescent protein, wherein the cell expresses the Cas9N protein and the Cas9^{C} fluorescent fusion protein and join them to form a full length Cas9^{FL} fluorescent fusion protein, and wherein the cell expresses the gRNA, and the gRNA directs the Cas9^{FL} fluorescent fusion protein to the target nucleic acid and produces fluorescent imaging of the target nucleic acid.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIGS. 1A-H depict that split-Cas9 retains full activity of Cas9^{FL} and enables AAV packaging with fusion domains. (FIG. 1A) SpCas9 (canonical PAM: NGG) broadly targets the human exome and transcriptional start sites (TSS), while orthologs suffer from restrictive PAMs (Sa: NNGRRT; St1: NNAGAAW; Nm: NNNNGATT). Sp* and Sa* denote engineered Cas9 variants and include non-canonical PAMs. (FIG. 1B) Schematic of split-Cas9 and AAV-Cas9. (FIG. 1C) Split-Cas9 achieves equivalent editing frequencies as Cas9^{FL} (one-way ANOVA). Each data point depicts mean±s.e.m. of 6 means (source data in FIG. 2C). (FIG. 1D) AAV-Cas9-gRNAs gene-edited myotubes. At each functional Cas9^{N}:Cas9^{C}, mutation frequency increased with AAV dose (one-way ANOVA), but began to plateau at ˜6% (n.s., not significant between 1E11 and 1E12). (FIG. 1E) AAV-Cas9-gRNAs (black) edited the Mstn gene in GC-1 spermatogonial cells, while AAV-Cas9-VPR-gRNAs (cyan) exhibited reduced endonucleolytic activity (Cas9^{N}:Cas9^{C}, 1:1) (n-way ANOVA). (FIG. 1F) Schematic of genome-editing and transcriptional regulation within a single system. A nuclease-active Cas9 is fused to a transcriptional activator domain. Cas9-mediated endonucleolytic DNA cleavage is programmed with a full-length gRNA, whereas Cas9-mediated transcriptional activation is programmed with a truncated gRNA. (FIG. 1G) AAV-Cas9-VPR-gRNAs upregulated target genes, as assessed by qRT-PCR (top, GC-1 cells; bottom, C2C12 myotubes; one-way ANOVA). (FIG. 1H) Transcriptional activation levels correlate inversely with basal gene expression levels. Data from GC-1 cells in red, data from C2C12 myotubes in black; closed dots denote with single-gRNA, and open circles denote with dual-gRNAs. *, P<0.05, ***, P<0.001, ANOVA followed by Holm-S̆idák test. Error bars denote s.e.m.

FIGS. 2A-D depict that split-Cas9 retains full biological activity of full-length Cas9. (FIG. 2A) SpCas9 consists of a bi-lobed structure (PDB IDs: 4OO8 and 4CMP) (Nishimasu, H. et al. Crystal structure of Cas9 in complex with guide RNA and target DNA. Cell 156, 935-949, doi:10.1016/j.cell.2014.02.001 (2014); Jinek, M. et al. Structures of Cas9 endonucleases reveal RNA-mediated conformational activation. Science 343, 1247997, doi:10.1126/science.1247997 (2014)), with the N- and C-termini of the disordered linker indicated. Cas9 is shown bound to the gRNA (red ribbon) and target DNA (blue ribbon). (FIG. 2B) Schematic of plasmids encoding split-Cas9. SMVP=promoter; IntN/IntC=split-inteins; NLS=nuclear localization signal; polyA=SV40 polyadenylation signal. (FIG. 2C) Split-Cas9 was tested against full-length Cas9 (Cas9^{FL}) on three endogenous genes, with or without co-translating P2A-turboGFP, by transfecting C2C12 cells with equal total mass amounts of Cas9 plasmids. Mutation frequencies induced by split-Cas9 and Cas9^{FL} are not significantly different across all three genes (one-way ANOVA) (400 ng of total Cas9 plasmids and 400 ng of total gRNAs plasmids). Left panels: Cas9 without P2A-turboGFP (n=3 independent transfections); Right panels: Cas9 with P2A-turboGFP (n=2 independent transfections). Error bars denote s.e.m. (FIG. 2D) Split-Cas9 targets Ai9 fibroblasts equivalently to Cas9^{FL}, activating tdTomato fluorescence by excision of the 3×Stop terminators cassette. Sparse tdTomato+ cells were observed with single-gRNA, or paired-gRNAs both targeting one side of 3×Stop (n=2). Td5 and TdL target 5′ of 3×Stop; Td3 and TdR target 3′ of 3×Stop. Gray=tdTomato. Scale bar, 200 μm.

FIGS. 3A-D depict that transduction of AAV-Cas9-gRNAs directs gene-editing in differentiated myotubes and tail-tip fibroblasts. (FIG. 3A) Schematic of AAV-Cas9-gRNAs. ITR=AAV inverted terminal repeat; SMVP and CASI=promoters; IntN/IntC=split-inteins; NLS=nuclear localization signal; polyA=SV40 polyadenylation signal. (FIG. 3B) Epifluorescence time course of AAV-Cas9^{C}-P2A-turboGFP expression, with onset by 1-2 days post-transduction. (FIG. 3C) Both unpurified AAV-Cas9-gRNAs-containing lysates (100 μl per well) or 1E10 (vg, vector genomes) of chloroform-ammonium sulfate purified AAV-Cas9-gRNAs edited the targeted endogenous loci in differentiated C2C12 myotubes. Cas9^{N}:Cas9c ratio of 1:1 was used. Each dot represents the mutation frequency detected per transduction per condition (P-values, one-tailed Wilcoxon rank-sum against no-gRNA controls, Bonferroni corrected). Red lines denote means±s.e.m. (FIG. 3D) Transduction of Ai9 tail-tip fibroblasts with 1E12 (total vg) of AAV-Cas9-gRNAs targeting the 3×Stop cassette induced excision-dependent fluorescence activation (n=2 transductions). gRNA pairs and Cas9^{N}:Cas9^{C}-P2A-turboGFP ratios are indicated. Td5 and TdL target 5′ of 3×Stop; Td3 and TdR target 3′ of 3×Stop. TdTomato was not observed in negative controls transduced with 6.7E11 (total vg) of Cas9^{C}-P2A-turboGFP only. Images were taken 7 days post-transduction. Scale bars, 500 μm.

FIGS. 4A-D depict postnatal genome-editing with AAV9-Cas9-gRNAs and transcriptional activation with AAV9-Cas9-VPR-gRNAs. (FIG. 4A) AAV9-Cas9-gRNAs targeting the endogenous Mstn gene or the 3×Stop cassette in neonatal mice. (FIG. 4B) Mutation frequency correlates with AAV transduction efficiency (Pearson's R=0.73, Spearman's ρ=0.74, P<0.05) (n=4 mice, 4E12 of AAV9-Cas9-gRNAs^{M3+4}). Horizontal dashed line=sequencing error rate; vertical dashed line=qPCR false positive rate. Error bars denote s.e.m. for sequencing and qPCR replicates. (FIG. 4C) AAV9-Cas9-gRNAs^{TdL+TdR}-edited tdTomato+ cells were detected in multiple organs of Ai9 mice (2 upper rows) (n=3 mice at 4E12), and absent in Ai9 mice injected with AAV9-Cas9-gRNAs^{M3+M4} (2 lower rows) (n=4 mice at 4E12). Gray=tdTomato. Scale bar, 5 mm. (FIG. 4D) AAV9-Cas9-VPR-gRNAs activated the target Pd-l1 and Cd47 genes in adult mice (FDR=0.05). Volcano plot shows total mRNA-sequencing of the same muscle samples used for qRT-PCR in FIG. 4E (n=3 mice per condition). (FIG. 4E) AAV9-Cas9-VPR-gRNAs activated the target Pd-l1 and Cd47 genes in adult skeletal muscles, as assessed by qRT-PCR and calculated as 2^{-ΔΔCt} (n=3 mice per group). Fold-change in gene expression was quantified between AAV9-Cas9-VPR-gRNAs-treated samples that differed only in the gRNA spacer sequences (one-tailed t-test). Samples treated with AAV9-Cas9-VPR-gRNAs and AAV9-turboRFP showed transcriptional alterations against samples treated with AAV9-turboRFP only, due to immunity-associated transcriptome perturbation. AAV-Cas9^{N}-gRNAs:AAV-Cas9^{C}-VPR ratio of 1:1 was used in all experiments. Error bars denote s.e.m.

FIGS. 5A-H depict that systemically delivered AAV9-Cas9-gRNAs genetically modify multiple organs, with editing frequency reflecting viral transduction efficiency. (FIG. 5A) Deep-sequencing of tissues indicates mean Mstn gene-targeting rates ranging from 7.8% to 0.25% (n=4 mice, 4E12 of AAV9-Cas9-gRNAs^{M3+M4}) (*, P<0.05, Wilcoxon rank-sum, Bonferroni corrected). Error bars denote s.e.m. Black dashed line denotes sequencing error. (FIG. 5B) Putative off-target sites were assessed by deep-sequencing. The bona fide off-target locus (chr16:+3906202) contains two mismatches (in red) against the on-target sequence. (n=4 mice, 4E12 of AAV9-Cas9-gRNAs^{M3+M4}; and n=2 control mice, 4E12 of AAV9-Cas9-gRNAs^{TdL+TdR} for determination of baseline sequencing error rates). (FIG. 5C) Recombinase-activated tdTomato fluorescence by AAV9-GFP-Cre (n=2, 2.5E11). Mean vg/dg shown. All examined cells within the liver, heart and muscle recombined, indicating ˜100% transduction efficiency within these organs. Within the testis, absence of tdTomato+ cells in the germline-residing seminiferous tubules argues against AAV9 transmission to the male germline. (FIG. 5D) Dual-AAV9s co-transduced multiple organs (n=2, 2E12 each of AAV9-GFP and AAV9-mCherry). (FIG. 5E) Triple-AAV9s co-transduction generated double edits on the same chromatin (n=4 mice co-injected with 2E12 of AAV9-Cas9^{C}-P2A-turboGFP, 1E12 of AAV9-Cas9N-gRNA^{M3}, and 1E12 of AAV9-Cas9N-gRNA^{M4}). M3 or M4, single-site edits; M3+M4, double-site edits; Precise excision, subset of M3+M4 with deletions delimited by the Cas9-gRNAs cut-sites. (FIG. 5F) AAV9-Cas9-gRNAs preferentially transduced the liver, heart and skeletal muscle (gastrocnemius and diaphragm) (***, P<0.001; Wilcoxon rank-sum, Bonferroni corrected) (n=7, 4E12). Red, means±s.e.m.; black dashed line with gray box, qPCR false positive rate (2.5 vg/dg) with s.d. (FIG. 5G) Transduction efficiency with 5E11 of AAV9-Cas9-gRNAs (**, P<0.01; ***, P<0.001; Wilcoxon rank-sum, Bonferroni corrected) (n=9). (FIG. 5H) Correlation of gene-targeting rates with vg/dg is maintained at lower dosage (n=2, 5E11 of AAV9-Cas9-gRNAs^{M3+M4}). Data from mice injected with 4E12 vg of AAV9-Cas9-gRNAs^{M3+M4}, as shown in FIG. 4B, is reproduced here for comparison.

FIG. 6 depicts whole-mount epifluorescence images from neonatal Ai9 mice injected systemically with AAV9-Cas9-gRNAs (5E11 vg) targeting the 3×Stop cassette and controls. Numerous tdTomato+ cells were observed in mice injected with AAV9-Cas9-gRNAs targeting the genomic 3×Stop cassette, but not in negative control vehicle-injected mice, indicating that fluorescence activation resulted from 3×Stop excision. TdTomato+ cells were also observed, at lower frequencies, in mice injected with AAV9s encoding two gRNAs both targeting one side of the 3×Stop cassette (AAV9-Cas9-gRNAs^{Td5+TdL} or AAV9-Cas9-gRNAs^{Td3+TdR}), suggesting the introduction of large deletions that removed the 3×Stop terminators. Gray=tdTomato.

FIGS. 7A-C depict that tissue sections from Ai9 mice injected with AAV9-Cas9-gRNAs. AAV9-Cas9-gRNAs^{TdL+TdR} (4E12 vg) transduced multiple organs, excising the 3×Stop genomic locus, as indicated by tdTomato activation in (FIG. 7A) liver, (FIG. 7B) heart, and (FIG. 7C) skeletal muscle. TdTomato+ cells were not detected in control mice injected with AAV9-Cas9-gRNAs^{M3+M4}. Scale bars, 500 μm.

FIGS. 8A-I depict that AAV9 and Cas9 activate immune responses. (FIG. 8A) Intramuscular Cas9-expression via AAV9-split-Cas9 injection or plasmid-Cas9^{FL} electroporation. (FIG. 8B) Intramuscular Cas9 expression induces lymphocyte infiltration in the draining inguinal and popliteal lymph nodes (n=4 mice per condition) (*, P<0.05; n-way ANOVA). Checkmarks denote injected vectors and conditions. (FIG. 8C) TCR-β CDR3 repertoires converged after Cas9-exposure, indicating Cas9-induced expansion of T-cell subsets (n=4; 6 pair-wise comparisons) (Welch's t-test, Bonferroni corrected). (FIG. 8D) Vβ16 CDR3 CASSLDRGQDTQYF is a public Cas9-responsive T-cell clonotype (Welch's t-test). Numbers in parentheses denote clonotypic rank within each TCR-β CDR3 repertoire after Cas9 re-stimulation. (FIG. 8E) Epitope mapping by M13 phage display (all Ig subclasses). (FIG. 8F) Cas9 epitopes from Cas9-exposed animals (top, n=4 electroporated; bottom, n=4 AAV9-delivered). Immunodominant epitopes reside in REC1 and PI domains (vertical dotted lines), and represented on Cas9 structure (PDB ID: 4CMP). Red, immunodominant epitopes; Black, private epitopes; Cyan, REC1; Pink, PI. P-values from Wald test, Benjamini-Hochberg adjusted for FDR=0.1. (FIG. 8G) Capsid epitopes from AAV9-exposed animals (n=8). Counts denote number of animals with capsid-specific antibodies, and red bars denote immunodominant epitopes. AAV9 capsid expresses as three isoforms (VP1/2/3). (FIG. 8H) Capsid residues within identified epitopes preferentially confer loss of viral blood persistency when mutated (Adachi, K., Enoki, T., Kawano, Y., Veraz, M. & Nakai, H. Drawing a high-resolution functional map of adeno-associated virus capsid by massively parallel sequencing. Nat Commun 5, 3075, doi:10.1038/ncomms4075 (2014)), suggesting their association with maintaining blood persistency. Each dot represents a double-alanine mutated AAV9 capsid variant, plotted according to its measured blood persistency and antigenicity of the residue. Red bar, mean. (FIG. 8I) Capsid residues within identified epitopes preferentially de-targets the liver when mutated (Adachi, K., Enoki, T., Kawano, Y., Veraz, M. & Nakai, H. Drawing a high-resolution functional map of adeno-associated virus capsid by massively parallel sequencing. Nat Commun 5, 3075, doi:10.1038/ncomms4075 (2014)), suggesting their association with hepatotropism. Each dot represents a double-alanine mutated AAV9 capsid variant, plotted according to its measured tropism and antigenicity of the residue. Blue bar, mean liver transduction efficiency; Magenta bar, mean global transduction efficiency, excluding the liver. Antigenicity, ranging from 0 to 8, denotes number of animals in which a particular residue is part of a linear epitope.

FIGS. 9A-D depict additional data for epitope-mapping and recoding of AAV9-CRISPR-Cas9. (FIG. 9A) Mapped epitopes for monoclonal (mAb) and polyclonal (pAb) Cas9-specific antibodies titrated at 200, 20, and 2 μg ml⁻¹. P-values from Wald test, Benjamini-Hochberg adjusted for FDR=0.1. (FIG. 9B) Known functional variants of Cas9 (Jinek, M. et al. Structures of Cas9 endonucleases reveal RNA-mediated conformational activation. Science 343, 1247997, doi:10.1126/science.1247997 (2014); Kleinstiver, B. P. et al. Engineered CRISPR-Cas9 nucleases with altered PAM specificities. Nature 523, 481-485, doi:10.1038/nature14592 (2015)) can be combined to recode identified epitopes. Recoded Cas9 retains endonucleolytic function. Ai9 fibroblasts were lipofected with wild-type or variant Cas9-encoding plasmids, programmed with the indicated gRNAs, and genomic excision-dependent tdTomato fluorescence was assayed 4 days post-transfection. Deletion of the epitope (Δ1126-1135) abolishes Cas9 activity. Scale bar, 500 μm. (FIG. 9C) AAV9-specific antibodies were elicited by two weeks post-injection, as determined by fluorescent immunoassay (FIAX). Two groups of mice injected with 4E12 vg AAV9-Cas9-VPR-gRNAs are shown, differing only in the gRNA spacers employed (**, P<0.01; one-way ANOVA, followed by Dunnett's test against vehicle-injected mice). (FIG. 9D) AAV9 capsid-specific epitopes reside predominantly on the capsid surface. Red bar, mean. Antigenicity, ranging from 0 to 8, denotes number of animals in which a particular residue is part of a linear epitope.

FIGS. 10A-D depict that AAV-CRISPR-Cas9 does not induce effector cytolysis seen with DNA electroporation. (FIG. 10A) AAV9-Cas9-VPR-gRNAs treatment does not elicit intramuscular IL-2 secretion or perforin release (n=3 mice per condition). Mice were targeted with 7 gRNAs against Mstn, Fst, PD-L1 and CD47 (gRNAs set 1) or with 3 gRNAs against Mstn and Fst (gRNAs set 2), all at 4E12 vg total of 1:1 AAV9-Cas9N-gRNAs:AAV-Cas9^{C}-VPR. All injections included 1E11 vg of AAV9-turboRFP to demarcate transduction. (FIG. 10B) IL-2 and perforin protein levels were elevated in muscles electroporated with Cas9-encoding DNA (n=3 mice per condition). (FIG. 10C) AAV9-Cas9-VPR-gRNAs did not induce detectable myofiber damage, as assessed by quantifying the fraction of centrally nucleated myofibers (n=3 mice per condition) (one-way ANOVA, followed by Dunnett's test against mice injected with 1E11 vg of AAV9-turboRFP). (FIG. 10D) Cellular damage that causes myofiber degeneration and repair typically results in centrally nucleated myofibers under histological examination. Part of a histological section is shown to depict the quantification method. Delivery of minicircle-Cas9^{FL} or pCAG-GFP via DNA electroporation induced an increase in the fraction of centrally nucleated myofibers, compared to controls electroporated with vehicle only. FK506 reduced but did not fully mitigate the elevated fraction of centrally nucleated myofibers (n=4 mice per condition) (*, P<0.05; **, P<0.01; ***, P<0.001; one-way ANOVA, followed by Tukey-Kramer test).

### DETAILED DESCRIPTION

Embodiments of the present disclosure are directed to a method of altering a target nucleic acid in a cell. In one embodiment, the method includes providing to the cell a first nucleic acid encoding a first portion of a Cas9 protein and a guide RNA (gRNA), providing to the cell a second nucleic acid encoding a second portion of the Cas9 protein and optionally a transcriptional regulator, wherein the cell expresses the first portion of the Cas9 protein, the gRNA and the second portion of the Cas9 protein or the second portion of the Cas9 and the transcriptional regulator fusion protein, wherein the first portion of the Cas9 protein and the second portion of the Cas9 protein, or the first portion of the Cas9 protein and the second portion of the Cas9 and the transcriptional regulator fusion protein are joined together to form the Cas9 protein or the Cas9 fusion protein, and wherein the gRNA and the Cas9 protein, or the gRNA and the Cas9 fusion protein form a co-localization complex with the target nucleic acid and alter the expression of the target nucleic acid. In some embodiments, the Cas9 protein is enzymatically active and the enzymatically active Cas9 protein cleaves the target nucleic acid in a site specific manner. In other embodiments, the gRNA can have full length or truncated spacer sequence. In certain embodiments, the gRNA having a truncated spacer sequence guides the Cas9 protein or the Cas9 fusion protein to the target nucleic acid and regulate the expression of the target nucleic acid without cleaving the target nucleic acid.

In some embodiments, the first nucleic acid and the second nucleic acid are delivered to the cell by separate vectors. In one embodiment, the first nucleic acid is delivered to the cell by a plasmid or adeno-associated virus. In another embodiment, the second nucleic acid is delivered to the cell by a plasmid or an adeno-associated virus.

In some embodiments, the first nucleic acid esters a first portion of the Cas9 protein having a first split-mtein and wherein the second nucleic acid encodes a second portion of the Cas9 protein having a second split-intein complementarity to the first split-intein and wherein the first portion of the Cas9 protein and the second portion of the Cas9 protein are joined together to form the Cas9 protein. In other embodiments, the first nucleic acid encodes a first portion of the Cas9 protein having a N-split-intein RmaIntN and wherein the second nucleic acid encodes a second portion of the Cas9 protein having a C-split-intein RmaIntC and wherein the first portion of the Cas9 protein and the second portion of the Cas9 protein are joined together to form the Cas9 protein. In certain embodiments, the first portion of the Cas9 protein is the N-terminal lobe of the Cas9 protein and the second portion of the Cas9 protein is the C-terminal lobe of the Cas9 protein. In certain embodiments, the first portion of the Cas9 protein is the N-terminal lobe of the Cas9 protein up to amino acid V713 and the second portion of the Cas9 protein is the C-terminal lobe of the Cas9 protein beginning at D714.

Embodiments of the present disclosure are directed to a method of altering a target nucleic acid in a cell of a subject. In one embodiment, the method includes delivering to the cell of the subject a first nucleic acid encoding a first portion of a Cas9 protein and a guide RNA (gRNA) wherein the first nucleic acid is within a first vector, delivering to the cell of the subject a second nucleic acid encoding a second portion of the Cas9 protein and optionally a transcriptional regulator wherein the second nucleic acid is within a second vector, wherein the cell expresses the first portion of the Cas9 protein, the gRNA and the second portion of the Cas9 protein or the second portion of the Cas9 and the transcriptional regulator fusion protein, wherein the first portion of the Cas9 protein and the second portion of the Cas9 protein, or the first portion of the Cas9 protein and the second portion of the Cas9 and the transcriptional regulator fusion protein are joined together to form the Cas9 protein or the Cas9 fusion protein, and wherein the gRNA and the Cas9 protein, or the gRNA and the Cas9 fusion protein form a co-localization complex with the target nucleic acid and alter the expression of the target nucleic acid. In certain embodiment, the Cas9 protein is enzymatically active and the enzymatically active Cas9 protein cleaves the target nucleic acid in a site specific manner. In some embodiments, the gRNA can have full length or truncated spacer sequence. In certain embodiments, the gRNA having a truncated spacer sequence guides the Cas9 protein or the Cas9 fusion protein to the target nucleic acid and regulate the expression of the target nucleic acid without cleaving the target nucleic acid.

In one embodiment, the first vector is a plasmid or adeno-associated virus. In another embodiment, the second vector is a plasmid or adeno-associated virus.

In some embodiments, the first nucleic acid encodes a first portion of the Cas9 protein having a first split-intein and wherein the second nucleic acid encodes a second portion of the Cas9 protein basing a second split-intein complementary to the first split-intein and wherein the first portion of die Cas9 protein and the second portion of the Cas9 protein are joined together to form the Cas9 protein. In other embodiments, the first nucleic acid encodes a first portion of the Cas9 protein having a N-split-intein RmaIntN and wherein the second nucleic acid encodes a second portion of the Cas9 protein having a C-split-intein RmaIntC and wherein the first portion of the Cas9 protein and the second portion of the Cas9 protein are joined together to form die Cas9 protein. In one embodiment, the first portion of the Cas9 protein is the N-terminal lobe of the Cas9 protein and the second portion of die Cas9 protein is the C-terminal lobe of the Cas9 protein. In another embodiment, the first portion of the Cas9 protein is the N-terminal lobe of die Cas9 protein up to amino acid V713 and the second portion of the Cas9 protein is the C-terminal lobe of the Cas9 protein beginning at D714.

In some embodiments, the vectors are delivered to the cell of the subject via various routes known to a skilled in the art including systemic, local, intravenous, intraperitoneal, intramuscular routes or via injection or electroporation. The subject of the disclosure includes human, patients or an animal. No overt cellular or tissue damage is observed when the vectors are adeno-associated viruses.

Embodiments of the present disclosure are directed to a method of modulating a target gene expression in a cell. In one embodiment, the method includes providing to the cell a first recombinant adeno-associated virus comprising a first nucleic acid encoding an N-terminal portion of the Cas9 protein (Cas9^{N}) and a gRNA, providing to the cell a second recombinant adeno-associated virus comprising a second nucleic acid encoding a fusion protein comprising a C-terminal portion of the Cas9 protein (Cas9^{C}) fused with a transcriptional regulator (TR), wherein the cell expresses the Cas9^{N} protein and the Cas9^{C}-TR fusion protein and join them to form a full length Cas9^{FL}-TR fusion protein, and wherein the cell expresses the gRNA and the gRNA directs the Cas9^{FL}-TR fusion protein to the target gene and modulates target gene expression.

In some embodiments, the transcriptional regulator is a transcriptional activator or a transcriptional repressor. In one embodiment, the transcriptional activator is VPR. In certain embodiments, the transcriptional regulator is a recruiter protein that recruits epigenetic modulators to the target gene. In exemplary embodiments, the gRNA has truncated spacer sequence and directs Cas9^{FL}-TR fusion protein binding to target DNA without cleaving the target DNA.

Embodiments of the present event disclosure are directed to a method of imaging a target nucleic acid in a cell. In one embodiment, the method includes providing to the cell a first recombinant adeno-associated virus comprising a first nucleic acid encoding an N-terminal portion of the Cas9 protein (Cas9^{N}) and a gRNA, providing to the cell a second recombinant adeno-associated virus comprising a second nucleic acid encoding a fusion protein comprising a C-terminal portion of the Cas9^{N} protein (Cas9^{C}) fused with a fluorescein protein, wherein the cell expresses the Cas9^{N} protein and the Cas9^{C} fluorescent fusion protein and join them to form a full length Cas9^{FL} fluorescent fusion protein, and wherein the cell expresses the gRNA, and the gRNA directs the Cas9^{FL} fluorescein fusion protein to the target nucleic acid and produces fluorescent imagine of the target nucleic acid.

In an exemplary embodiment, the Cas9 is a Type II CRISPR system Cas9. In some embodiments, the Cas9 protein is an enzymatically active Cas9 protein, a Cas9 protein nickase or a nuclease null Cas9 protein.

In some embodiments, the Cas9^{N} protein and the Cas9^{C}-TR fusion protein are joined by split inteins.

The cell according to some embodiments of the present disclosure is a eukaryotic cell or a prokaryotic cell. In certain embodiments, the cell is a bacteria cell, a yeast cell, a mammalian cell, a human cell, a plant cell or an animal cell. In some embodiments, the cell is in vitro, in vivo or ex vivo.

The split Cas9 methods described herein are useful in CRISPR-related methods where Cas9 and a guide RNA are used to colocalize the Cas9 and the guide RNA to a target nucleic acid sequence. Accordingly, embodiments of the present disclosure are based on the use of RNA guided DNA binding proteins, such as Cas9, to co-localize with guide RNA at a target DNA site. Such DNA binding proteins are readily known to those of skill in the art to bind to DNA for various purposes. Such DNA binding proteins may be naturally occurring. DNA binding proteins included within the scope of the present disclosure include those which may be guided by RNA, referred to herein as guide RNA. According to one aspect, the guide RNA is between about 10 to about 500 nucleotides. According to one aspect, the RNA is between about 20 to about 100 nucleotides. According to this aspect, the guide RNA and the RNA guided DNA binding protein form a co-localization complex at the DNA.

DNA binding proteins having nuclease activity are known to those of skill in the art, and include naturally occurring DNA binding proteins having nuclease activity, such as Cas9 proteins present, for example, in Type II CRISPR systems. Such Cas9 proteins and Type II CRISPR systems are well documented in the art. See Makarova et al., Nature Reviews, Microbiology, Vol. 9, June 2011, pp. 467-477 including all supplementary information hereby incorporated by reference in its entirety. Exemplary Cas include S. pyogenes Cas9 (SpCas9), S. aureus Cas9 (SaCas9) and S. thermophilus Cas9 (StCas9). Additional exemplary CRISPR systems include Cpf1 proteins for RNA-guided genome-editing. See Zetsche, B. et al. Cpf1 is a single RNA-guided endonuclease of a class 2 CRISPR-Cas system. Cell, 2015, 163, 759-771. Additional exemplary nucleic-acid guided systems include argonaute proteins for DNA-guided genome-editing. See Gao F, Shen X Z, Jiang F, Wu Y, Han C, DNA-guided genome editing using the Natronobacterium gregoryi Argonaute, Nat Biotechnol., 2016 May 2. doi: 10.1038/nbt.3547 hereby incorporated by reference in its entirety.

Bacterial and archaeal CRISPR-Cas systems rely on short guide RNAs in complex with Cas proteins to direct degradation of complementary sequences present within invading foreign nucleic acid. See Deltcheva, E. et al. CRISPR RNA maturation by trans-encoded small RNA and host factor RNase III. Nature 471, 602-607 (2011); Gasiunas, G., Barrangou, R., Horvath, P. & Siksnys, V. Cas9-crRNA ribonucleoprotein complex mediates specific DNA cleavage for adaptive immunity in bacteria. Proceedings of the National Academy of Sciences of the United States of America 109, E2579-2586 (2012); Jinek, M. et al. A programmable dual-RNA-guided DNA endonuclease in adaptive bacterial immunity. Science 337, 816-821 (2012); Sapranauskas, R. et al. The Streptococcus thermophilus CRISPR/Cas system provides immunity in Escherichia coli. Nucleic acids research 39, 9275-9282 (2011); and Bhaya, D., Davison, M. & Barrangou, R. CRISPR-Cas systems in bacteria and archaea: versatile small RNAs for adaptive defense and regulation. Annual review of genetics 45, 273-297 (2011). A recent in vitro reconstitution of the S. pyogenes type II CRISPR system demonstrated that crRNA (“CRISPR RNA”) fused to a normally trans-encoded tracrRNA (“trans-activating CRISPR RNA”) is sufficient to direct Cas9 protein to sequence-specifically cleave target DNA sequences matching the crRNA. Expressing a gRNA complementary to a target site results in Cas9 recruitment and degradation of the target DNA. See H. Deveau et al., Phage response to CRISPR-encoded resistance in Streptococcus thermophilus. Journal of Bacteriology 190, 1390 (February, 2008).

Two classes of CRISPR systems are generally known and are referred to as class 1 and class 2. Class 1 systems can be further classified into types I, III, and IV, while class 2 systems can be further classified into type II and V. According to one aspect, a particular useful enzyme according to the present disclosure to cleave dsDNA is the single effector enzyme, Cas9, common to Type II. See K. S. Makarova et al., Evolution and classification of the CRISPR-Cas systems. Nature reviews. Microbiology 9, 467 (June, 2011) hereby incorporated by reference in its entirety. Within bacteria, the Type II effector system consists of a long pre-crRNA transcribed from the spacer-containing CRISPR locus, the multifunctional Cas9 protein, and a tracrRNA important for gRNA processing. The tracrRNAs hybridize to the repeat regions separating the spacers of the pre-crRNA, initiating dsRNA cleavage by endogenous RNase III, which is followed by a second cleavage event within each spacer by Cas9, producing mature crRNAs that remain associated with the tracrRNA and Cas9. TracrRNA-crRNA fusions are contemplated for use in the present methods.

According to one aspect, the enzyme of the present disclosure, such as Cas9 unwinds the DNA duplex and searches for sequences matching the crRNA to cleave. Target recognition occurs upon detection of complementarity between a “protospacer” sequence in the target DNA and the remaining spacer sequence in the crRNA. Importantly, Cas9 cuts the DNA only if a correct protospacer-adjacent motif (PAM) is also present at the 3′ end. According to certain aspects, different protospacer-adjacent motif can be utilized. For example, the S. pyogenes system requires an NGG sequence, where N can be any nucleotide. S. thermophilus Type II systems require NGGNG (see P. Horvath, R. Barrangou, CRISPR/Cas, the immune system of bacteria and archaea. Science 327, 167 (Jan. 8, 2010) hereby incorporated by reference in its entirety and NNAGAAW (see H. Deveau et al., Phage response to CRISPR-encoded resistance in Streptococcus thermophilus. Journal of bacteriology 190, 1390 (February, 2008) hereby incorporated by reference in its entirety), respectively, while different S. mutans systems tolerate NGG or NAAR (see J. R. van der Ploeg, Analysis of CRISPR in Streptococcus mutans suggests frequent occurrence of acquired immunity against infection by M102-like bacteriophages. Microbiology 155, 1966 (June, 2009) hereby incorporated by reference in its entirety. Bioinformatic analyses have generated extensive databases of CRISPR loci in a variety of bacteria that may serve to identify additional useful PAMs and expand the set of CRISPR-targetable sequences (see M. Rho, Y. W. Wu, H. Tang, T. G. Doak, Y. Ye, Diverse CRISPRs evolving in human microbiomes. PLoS genetics 8, e1002441 (2012) and D. T. Pride et al., Analysis of streptococcal CRISPRs from human saliva reveals substantial sequence diversity within and between subjects over time. Genome research 21, 126 (January, 2011); Kleinstiver B P, Prew M S, Tsai S Q, Topkar V V, Nguyen N T, Zheng Z, Gonzales A P, Li Z, Peterson R T, Yeh J R, Aryee M J, Joung J K, Engineered CRISPR-Cas9 nucleases with altered PAM specificities, Nature. 2015 Jul. 23; 523(7561):481-5; Kleinstiver B P, Prew M S, Tsai S Q, Nguyen N T, Topkar V V, Zheng Z, Joung J K, Broadening the targeting range of Staphylococcus aureus CRISPR-Cas9 by modifying PAM recognition, Nat Biotechnol. 2015 December; 33(12):1293-1298, each of which are hereby incorporated by reference in their entireties. According to one aspect, a particular useful enzyme according to the present disclosure to cleave dsDNA is the single effector enzyme, Cpf1, belonging to Type V. See Zetsche, B. et al. Cpf1 is a single RNA-guided endonuclease of a class 2 CRISPR-Cas system. Cell, 2015, 163, 759-771.

Exemplary DNA binding proteins having nuclease activity function to nick or cut double stranded DNA. Such nuclease activity may result from the DNA binding protein having one or more polypeptide sequences exhibiting nuclease activity. Such exemplary DNA binding proteins may have two separate nuclease domains with each domain responsible for cutting or nicking a particular strand of the double stranded DNA. Exemplary polypeptide sequences having nuclease activity known to those of skill in the art include the McrA-HNH nuclease related domain and the RuvC-like nuclease domain. Accordingly, exemplary DNA binding proteins are those that in nature contain one or more of the McrA-HNH nuclease related domain and the RuvC-like nuclease domain.

In S. pyogenes, Cas9 generates a blunt-ended double-stranded break 2-4 bp upstream of the protospacer-adjacent motif (PAM) via a process mediated by two catalytic domains in the protein: an HNH domain that cleaves the complementary strand of the DNA and a RuvC-like domain that cleaves the non-complementary strand. See Jinek et al., Science 337, 816-821 (2012) hereby incorporated by reference in its entirety. Cas9 proteins are known to exist in many Type II CRISPR systems including the following as identified in the supplementary information to Makarova et al., Nature Reviews, Microbiology, Vol. 9, June 2011, pp. 467-477: Methanococcus maripaludis C7; Corynebacterium diphtheriae; Corynebacterium efficiens YS-314; Corynebacterium glutamicum ATCC 13032 Kitasato; Corynebacterium glutamicum ATCC 13032 Bielefeld; Corynebacterium glutamicum R; Corynebacterium kroppenstedtii DSM 44385; Mycobacterium abscessus ATCC 19977; Nocardia farcinica IFM10152; Rhodococcus erythropolis PR4; Rhodococcus jostii RHA1; Rhodococcus opacus B4 uid36573; Acidothermus cellulolyticus 11B; Arthrobacter chlorophenolicus A6; Kribbella flavida DSM 17836 uid43465; Thermomonospora curvata DSM 43183; Bifidobacterium dentium Bd1; Bifidobacterium longum DJO10A; Slackia heliotrinireducens DSM 20476; Persephonella marina EX H1; Bacteroides fragilis NCTC 9434; Capnocytophaga ochracea DSM 7271; Flavobacterium psychrophilum JIP02 86; Akkermansia muciniphila ATCC BAA 835; Roseiflexus castenholzii DSM 13941; Roseiflexus RS1; Synechocystis PCC6803; Elusimicrobium minutum Pei191; uncultured Termite group 1 bacterium phylotype Rs D17; Fibrobacter succinogenes S85; Bacillus cereus ATCC 10987; Listeria innocua; Lactobacillus casei; Lactobacillus rhamnosus GG; Lactobacillus salivarius UCC118; Streptococcus agalactiae A909; Streptococcus agalactiae NEM316; Streptococcus agalactiae 2603; Streptococcus dysgalactiae equisimilis GGS 124; Streptococcus equi zooepidemicus MGCS10565; Streptococcus gallolyticus UCN34 uid46061; Streptococcus gordonii Challis subst CH1; Streptococcus mutans NN2025 uid46353; Streptococcus mutans; Streptococcus pyogenes M1 GAS; Streptococcus pyogenes MGAS5005; Streptococcus pyogenes MGAS2096; Streptococcus pyogenes MGAS9429; Streptococcus pyogenes MGAS10270; Streptococcus pyogenes MGAS6180; Streptococcus pyogenes MGAS315; Streptococcus pyogenes SSI-1; Streptococcus pyogenes MGAS10750; Streptococcus pyogenes NZ131; Streptococcus thermophiles CNRZ1066; Streptococcus thermophiles LMD-9; Streptococcus thermophiles LMG 18311; Clostridium botulinum A3 Loch Maree; Clostridium botulinum B Eklund 17B; Clostridium botulinum Ba4 657; Clostridium botulinum F Langeland; Clostridium cellulolyticum H10; Finegoldia magna ATCC 29328; Eubacterium rectale ATCC 33656; Mycoplasma gallisepticum; Mycoplasma mobile 163K; Mycoplasma penetrans; Mycoplasma synoviae 53; Streptobacillus moniliformis DSM 12112; Bradyrhizobium BTAi1; Nitrobacter hamburgensis X14; Rhodopseudomonas palustris BisB18; Rhodopseudomonas palustris BisB5; Parvibaculum lavamentivorans DS-1; Dinoroseobacter shibae DFL 12; Gluconacetobacter diazotrophicus Pal 5 FAPERJ; Gluconacetobacter diazotrophicus Pal 5 JGI; Azospirillum B510 uid46085; Rhodospirillum rubrum ATCC 11170; Diaphorobacter TPSY uid29975; Verminephrobacter eiseniae EF01-2; Neisseria meningitides 053442; Neisseria meningitides alpha14; Neisseria meningitides Z2491; Desulfovibrio salexigens DSM 2638; Campylobacter jejuni doylei 269 97; Campylobacter jejuni 81116; Campylobacter jejuni; Campylobacter lari RM2100; Helicobacter hepaticus; Wolinella succinogenes; Tolumonas auensis DSM 9187; Pseudoalteromonas atlantica T6c; Shewanella pealeana ATCC 700345; Legionella pneumophila Paris; Actinobacillus succinogenes 130Z; Pasteurella multocida; Francisella tularensis novicida U112; Francisella tularensis holarctica; Francisella tularensis FSC 198; Francisella tularensis tularensis; Francisella tularensis WY96-3418; and Treponema denticola ATCC 35405. The Cas9 protein may be referred by one of skill in the art in the literature as Csn1. An exemplary S. pyogenes Cas9 protein sequence is shown below. See Deltcheva et al., Nature 471, 602-607 (2011) hereby incorporated by reference in its entirety.

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKIEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

According to one aspect, the specificity of gRNA-directed Cas9 cleavage is used as a mechanism for genome engineering. According to one aspect, hybridization of the gRNA need not be 100 percent in order for the enzyme to recognize the gRNA/DNA hybrid and affect cleavage. Some off-target activity could occur. For example, the S. pyogenes system tolerates mismatches in the first 6 bases out of the 20 bp mature spacer sequence in vitro. According to one aspect, greater stringency may be beneficial in vivo when potential off-target sites matching (last 14 bp) NGG do not exist within the human reference genome for the gRNAs.

According to certain aspects, specificity may be improved. When interference is sensitive to the melting temperature of the gRNA-DNA hybrid, AT-rich target sequences may have fewer off-target sites. Carefully choosing target sites to avoid pseudo-sites with at least 14 bp matching sequences elsewhere in the genome may improve specificity. According to certain aspect, the gRNAs can be designed to include 16-18 nucleotide spacers, which increases specificity while retaining Cas9 endonucleolytic activity (Fu Y, Sander J D, Reyon D, Cascio V M, Joung J K, Improving CRISPR-Cas nuclease specificity using truncated guide RNAs, Nat Biotechnol., 2014 March; 32(3):279-84, hereby incorporated by reference in its entirety). The use of a Cas9 variant requiring a longer PAM sequence may reduce the frequency of off-target sites. Directed evolution may improve Cas9 specificity to a level sufficient to completely preclude off-target activity, ideally requiring a perfect 20 bp gRNA match with a minimal PAM. Accordingly, modification to the Cas9 protein is a representative embodiment of the present disclosure. CRISPR systems useful in the present disclosure are described in R. Barrangou, P. Horvath, CRISPR: new horizons in phage resistance and strain identification. Annual review of food science and technology 3, 143 (2012) and B. Wiedenheft, S. H. Sternberg, J. A. Doudna, RNA-guided genetic silencing systems in bacteria and archaea. Nature 482, 331 (Feb. 16, 2012) each of which are hereby incorporated by reference in their entireties.

Guide RNAs useful in the disclosed methods include those having a spacer sequence, a tracr mate sequence and a tracr sequence, with the spacer sequence being between about 16 to about 20 nucleotides in length and with the tracr sequence being between about 60 to about 500 nucleotides in length and with a portion of the tracr sequence being hybridized to the tracr mate sequence and with the tracr mate sequence and the tracr sequence being linked by a linker nucleic acid sequence of between about 4 to about 6 nucleotides. crRNA-tracrRNA fusions are contemplated as exemplary guide RNA.

According to certain aspects, the DNA binding protein is altered or otherwise modified to inactivate the nuclease activity. Such alteration or modification includes altering one or more amino acids to inactivate the nuclease activity or the nuclease domain. Such modification includes removing the polypeptide sequence or polypeptide sequences exhibiting nuclease activity, i.e. the nuclease domain, such that the polypeptide sequence or polypeptide sequences exhibiting nuclease activity, i.e. nuclease domain, are absent from the DNA binding protein. Other modifications to inactivate nuclease activity will be readily apparent to one of skill in the art based on the present disclosure. Accordingly, a nuclease-null DNA binding protein includes polypeptide sequences modified to inactivate nuclease activity or removal of a polypeptide sequence or sequences to inactivate nuclease activity. The nuclease-null DNA binding protein retains the ability to bind to DNA even though the nuclease activity has been inactivated. Accordingly, the DNA binding protein includes the polypeptide sequence or sequences required for DNA binding but may lack the one or more or all of the nuclease sequences exhibiting nuclease activity. Accordingly, the DNA binding protein includes the polypeptide sequence or sequences required for DNA binding but may have one or more or all of the nuclease sequences exhibiting nuclease activity inactivated.

According to one aspect, a DNA binding protein having two or more nuclease domains may be modified or altered to inactivate all but one of the nuclease domains. Such a modified or altered DNA binding protein is referred to as a DNA binding protein nickase, to the extent that the DNA binding protein cuts or nicks only one strand of double stranded DNA. When guided by RNA to DNA, the DNA binding protein nickase is referred to as an RNA guided DNA binding protein nickase.

An exemplary DNA binding protein is an RNA guided DNA binding protein nuclease of a Type II CRISPR System, such as a Cas9 protein or modified Cas9 or homolog of Cas9 or ortholog of Cas9. An exemplary DNA binding protein is an RNA guided DNA binding protein nuclease of a Type V CRISPR System, such as a Cpf1 protein or modified Cpf1 or homolog of Cpf1 or ortholog of Cpf1. An exemplary DNA binding protein is a Cas9 protein nickase. An exemplary DNA binding protein is an RNA guided DNA binding protein of a Type II CRISPR System which lacks nuclease activity. An exemplary DNA binding protein is a nuclease-null Cas9 protein. An exemplary DNA binding protein is an RNA guided DNA binding protein of a Type V CRISPR System which lacks nuclease activity. An exemplary DNA binding protein is a nuclease-null Cpf1 protein.

According to one aspect, the Cas9 protein, Cas9 protein nickase or nuclease null Cas9 includes homologs and orthologs thereof which retain the ability of the protein to bind to the DNA and be guided by the RNA. According to one aspect, the Cas9 protein includes the sequence as set forth for naturally occurring Cas9 from S. pyogenes and protein sequences having at least 30%, 40%, 50%, 60%, 70%, 80%, 90%, 95%, 98% or 99% homology thereto and being a DNA binding protein, such as an RNA guided DNA binding protein.

According to one aspect, an engineered Cas9-gRNA system is provided wherein one or more of functional groups or function-conferring domains such as FokI heterodimers (see Tsai, S. Q., et al., Dimeric CRISPR RNA-guided FokI nucleases for highly specific genome editing. Nat Biotechnol, 2014. 32(6): p. 569-76, and Guilinger, J. P., D. B. Thompson, and D. R. Liu, Fusion of catalytically inactive Cas9 to FokI nuclease improves the specificity of genome modification. Nat Biotechnol, 2014. 32(6): p. 577-82.), transcriptional regulators (see Gilbert, L. A., et al., CRISPR-mediated modular RNA-guided regulation of transcription in eukaryotes. Cell, 2013. 154(2): p. 442-51, Mali, P., et al., CAS9 transcriptional activators for target specificity screening and paired nickases for cooperative genome engineering. Nat Biotechnol, 2013. 31(9): p. 833-8, Perez-Pinera, P., et al., RNA-guided gene activation by CRISPR-Cas9-based transcription factors. Nat Methods, 2013. 10(10): p. 973-6, and Cheng, A. W., et al., Multiplexed activation of endogenous genes by CRISPR-on, an RNA-guided transcriptional activator system. Cell Res, 2013. 23(10): p. 1163-71), fluorescent proteins (see Gilbert, L. A., et al., CRISPR-mediated modular RNA-guided regulation of transcription in eukaryotes. Cell, 2013. 154(2): p. 442-51 and Chen, B., et al., Dynamic imaging of genomic loci in living human cells by an optimized CRISPR/Cas system. Cell, 2013. 155(7): p. 1479-91), protein-protein interacting-domains (see Tanenbaum, M. E., et al., A protein-tagging system for signal amplification in gene expression and fluorescence imaging. Cell, 2014. 159(3): p. 635-46), nucleotide base editors (see Komor, A. C., Kim, Y. B., Packer, M. S., Zuris, J. A. & Liu, D. R. Programmable editing of a target base in genomic DNA without double-stranded DNA cleavage. Nature, doi:10.1038/nature17946 (2016)), and degradation tags are attached to either the Cas9 protein or the gRNA or both for delivery to a target nucleic acid.

According to one aspect, the transcriptional regulator protein or domain is a transcriptional activator. In an exemplary embodiment, the transcriptional activator is VPR. According to one aspect, the transcriptional regulator protein or domain upregulates expression of the target nucleic acid. According to one aspect, the transcriptional regulator protein or domain is a transcriptional repressor. According to one aspect, the transcriptional regulator protein or domain downregulates expression of the target nucleic acid. Transcriptional activators and transcriptional repressors can be readily identified by one of skill in the art based on the present disclosure.

According to one aspect, two or more guide RNAs are provided with each guide RNA being complementary to an adjacent site in the DNA target nucleic acid. At least one RNA guided DNA binding protein nickase is provided and being guided by the two or more RNAs, wherein the at least one RNA guided DNA binding protein nickase co-localizes with the two or more RNAs to the DNA target nucleic acid and nicks the DNA target nucleic acid resulting in two or more adjacent nicks. According to certain aspects, the two or more adjacent nicks are on the same strand of the double stranded DNA. According to one aspect, the two or more adjacent nicks are on the same strand of the double stranded DNA and result in homologous recombination. According to one aspect, the two or more adjacent nicks are on different strands of the double stranded DNA. According to one aspect, the two or more adjacent nicks are on different strands of the double stranded DNA and create double stranded breaks. According to one aspect, the two or more adjacent nicks are on different strands of the double stranded DNA and create double stranded breaks resulting in nonhomologous end joining. According to one aspect, the two or more adjacent nicks are on different strands of the double stranded DNA and are offset with respect to one another. According to one aspect, the two or more adjacent nicks are on different strands of the double stranded DNA and are offset with respect to one another and create double stranded breaks. According to one aspect, the two or more adjacent nicks are on different strands of the double stranded DNA and are offset with respect to one another and create double stranded breaks resulting in nonhomologous end joining. According to one aspect, the two or more adjacent nicks are on different strands of the double stranded DNA and create double stranded breaks resulting in fragmentation of the target nucleic acid thereby preventing expression of the target nucleic acid.

According to certain aspects, binding specificity of the RNA guided DNA binding protein may be increased according to methods described herein. According to one aspect, off-set nicks are used in methods of genome-editing (see Mali, P. et al. CAS9 transcriptional activators for target specificity screening and paired nickases for cooperative genome engineering. Nature biotechnology, 31, 833-838, doi:10.1038/nbt.2675 (2013) hereby incorporated by reference in its entirety). A large majority of nicks seldom result in NHEJ events, (see Certo et al., Nature Methods 8, 671-676 (2011) hereby incorporated by reference in its entirety) thus minimizing the effects of off-target nicking. In contrast, inducing off-set nicks to generate double stranded breaks (DSBs) is highly effective at inducing gene disruption. According to certain aspects, 5′ overhangs generate more significant NHEJ events as opposed to 3′ overhangs. Similarly, 3′ overhangs favor HR over NHEJ events, although the total number of HR events is significantly lower than when a 5′ overhang is generated. Accordingly, methods are provided for using nicks for homologous recombination and off-set nicks for generating double stranded breaks to minimize the effects of off-target Cas9-gRNA activity.

Target nucleic acids include any nucleic acid sequence to which a co-localization complex as described herein can be useful to either cut, nick, regulate or bind. Target nucleic acids include genes. For purposes of the present disclosure, DNA, such as double stranded DNA, can include the target nucleic acid and a co-localization complex can bind to or otherwise co-localize with the DNA at or adjacent or near the target nucleic acid and in a manner in which the co-localization complex may have a desired effect on the target nucleic acid. Such target nucleic acids can include endogenous (or naturally occurring) nucleic acids and exogenous (or foreign) nucleic acids. One of skill based on the present disclosure will readily be able to identify or design guide RNAs and Cas9 proteins which co-localize to a DNA including a target nucleic acid. One of skill will further be able to identify transcriptional regulator proteins or domains which likewise co-localize to a DNA including a target nucleic acid. DNA includes genomic DNA, mitochondrial DNA, viral DNA or exogenous DNA. CRISPR Cas9 system can also be programmed to target RNA (See, O'Connell M R, Oakes B L, Sternberg S H, East-Seletsky A, Kaplan M, Doudna J A, Programmable RNA recognition and cleavage by CRISPR/Cas9, Nature, 2014, December 11; 516(7530):263-6; Nelles D A, Fang M Y, O'Connell M, Xu J L, Markmiller S J, Doudna J A, Yeo G W, Programmable RNA Tracking in Live Cells with CRISPR/Cas9, Cell, 2016, April 7:165(2) 488-96, each of which is hereby incorporated by reference in its entirety).

Foreign nucleic acids (i.e. those which are not part of a cell's natural nucleic acid composition) may be introduced into a cell using any method known to those skilled in the art for such introduction. Such methods include transfection, transduction, viral transduction, microinjection, electroporation, lipofection, nucleofection, nanoparticle bombardment, transformation, conjugation and the like One of skill in the art will readily understand and adapt such methods using readily identifiable literature sources.

Transcriptional regulator proteins or domains which are transcriptional activators or transcriptional repressors may be readily identifiable, by those skilled in the art based on the present disclosure

Vectors used to deliver the nucleic acids to cells as described herein include vectors known to those of skill in the art and used for such purposes. Certain exemplary vectors may be plasmids or adeno-associated viruses known to those of skill in the art. AAVs are highly prevalent within the human population (see Gao, G., et al., Clades of Adeno-associated viruses are widely disseminated in human tissues J Virol. 2004. 78(12): p. 6381-8, and Boutin. S., et al., Prevalence of serum IgG and neutralizing factors against adeno-associated virus (AAV) types 1, 2, 5, 6, 8, and 9 in the healthy population, implications forgone therapy using AAV vectors. Hum Gene Ther. 2010. 21(6): p. 704-12) and are useful as viral vectors. Many serotypes exist, each with different tropism for tissue types (see Zincarelli, C., et al., Analysis of AAV serotypes 1-9 mediated gene expression and tropism in mice after systemic injection. Mol Ther, 2008. 16(6): p. 1073-80), which allows specific tissues to be preferentially targeted with appropriate pseudotyping. Some serotypes, such as serotypes 8, 9, and rh10, transduce the mammalian body. See Zincarelli, C., et al. Analysis of AAV serotypes 1-9 mediated gene expression and tropism in mice after systemic injection. Mol Ther, 2008. 16(6): p. 1073-80, Inagaki, K., et al., Robust systemic transduction with AAV9 vectors in mice: efficient global cardiac gene transfer superior to that of AAV8. Mol Ther, 2006. 14(1): p. 45-53, Keeler, A. M., et al., Long-term correction of very long-chain acyl-coA dehydrogenase deficiency in mice using AAV9 gene therapy. Mol Ther, 2012. 20(6): p. 1131-8, Gray, S. J., et al., Preclinical differences of intravascular AAV9 delivery to neurons and glia: a comparative study of adult mice and nonhuman primates. Mol Ther, 2011. 19(6): p. 1058-69, Okada, H., et al., Robust Long-term Transduction of Common Marmoset Neuromuscular Tissue With rAAV1 and rAAV9. Mol Ther Nucleic Acids, 2013. 2: p. e95, and Foust, K. D., et al., Intravascular AAV9 preferentially targets neonatal neurons and adult astrocytes. Nat Biotechnol, 2009. 27(1): p. 59-65. AAV9 has been demonstrated to cross the blood-brain barrier (see Foust, K. D., et al., Intravascular AAV9 preferentially targets neonatal neurons and adult astrocytes. Nat Biotechnol, 2009. 27(1): p. 59-65, and Rahim, A. A., et al., Intravenous administration of AAV2/9 to the fetal and neonatal mouse leads to differential targeting of CNS cell types and extensive transduction of the nervous system. FASEB J, 2011. 25(10): p. 3505-18) that is inaccessible to many viral vectors and biologics. Certain AAVs have a payload of 4.7-5.0 kb (including viral inverted terminal repeats (ITRs), which are required in cis for viral packaging). See Wu, Z., H. Yang, and P. Colosi, Effect of genome size on AAV vector packaging. Mol Ther, 2010. 18(1): p. 80-6 and Dong, J. Y., P. D. Fan, and R. A. Frizzell, Quantitative analysis of the packaging capacity of recombinant adeno-associated virus. Hum Gene Ther, 1996. 7(17): p. 2101-12.

Delivery methods commonly used in research, such as lentiviruses, adenoviruses, or nucleic-acid-complexes, exhibit substantial immunogenic and cytotoxic properties, which can further compound the immunogenicity from ectopic transgene-expression. Furthermore, these approaches generally lack the capacity for targeting of specific tissues and for robust full-body delivery. To simultaneously minimize pathological impacts and enable systemic genome editing, CRISPR was delivered via adeno-associated viruses (AAVs). AAVs are prevalent within human populations (see Gao, G., et al., Clades of Adeno-associated viruses are widely disseminated in human tissues. J Virol, 2004. 78(12): p. 6381-8), and there have been no established cases of pathology associated with AAV infection, making them one of the most promising vectors currently used in clinical trials. Moreover, tissue-targeting is easily accomplished by pseudotyping to AAV serotypes with suitable tropism. Of interest is AAV serotype 9, which robustly transduces multiple cell types in the body (see Zincarelli, C., et al., Analysis of AAV serotypes 1-9 mediated gene expression and tropism in mice after systemic injection. Mol Ther, 2008. 16(6): p. 1073-80. and Foust, K. D., et al., Intravascular AAV9 preferentially targets neonatal neurons and adult astrocytes. Nat Biotechnol, 2009. 27(1): p. 59-65.) and crosses endothelial barriers (e.g. blood-brain barrier, see Foust, K. D., et al., Intravascular AAV9 preferentially targets neonatal neurons and adult astrocytes. Nat Biotechnol, 2009. 27(1): p. 59-65 and Zhang, H. et al. Several rAAV vectors efficiently cross the blood-brain barrier and transduce neurons and astrocytes in the neonatal mouse central nervous system. Mol Ther, 2011 19(8): p. 1440-1448) that block access by other delivery vectors. Together, AAV and CRISPR present an enticing combination for achieving systemic gene-editing, but a key obstacle has been the limited capacity of AAV for packaging exogenous sequences (<4.7 kb). Dong, J. Y., P. D. Fan, and R. A. Frizzell, Quantitative analysis of the packaging capacity of recombinant adeno-associated virus. Hum Gene Ther, 1996. 7(17): p. 2101-12. Of the various Cas9 orthologs that have been co-opted for genome engineering (ST1, Nm, Sa) (see Ran, F. A. et al. In vivo genome editing using Staphylococcus aureus Cas9. Nature, 2015. 520: p. 186-91 and Esvelt, K. M., et al., Orthogonal Cas9 proteins for RNA-guided gene regulation and editing. Nat Methods, 2013. 10(11): p. 1116-21), Sp Cas9 has a least restrictive PAM and most consistent efficacy, but its size (4.2 kb) makes packaging into AAV challenging, necessitating use of a limited repertoire of compact regulatory elements (<500 bp) (see Swiech, L., et al., In vivo interrogation of gene function in the mammalian brain using CRISPR-Cas9. Nat Biotechnol, 2014 and Senis, E. et al. CRISPR/Cas9-mediated genome engineering: an adeno-associated viral (AAV) vector toolbox. Biotechnology journal, 2014, 9: p. 1402-1412; Long C, Amoasii L, Mireault A A, McAnally J R, Li H, Sanchez-Ortiz E, Bhattacharyya S, Shelton J M, Bassel-Duby R, Olson E N, Postnatal genome editing partially restores dystrophin expression in a mouse model of muscular dystrophy, Science, 2016 January 22; 351(6271):400-3; Yang Y, Wang L, Bell P, McMenamin D, He Z, White J, Yu H, Xu C, Morizono H, Musunuru K, Batshaw M L, Wilson J M, A dual AAV system enables the Cas9-mediated correction of a metabolic liver disease in newborn mice, Nat Biotechnol., 2016, March; 34(3):334-8; each of which are hereby incorporated by reference in its entirety), and precluding the fusion of function-conferring domains. The Cas9 protein was re-engineered to eliminate this obstacle.

According to certain aspects, two or more portions of a Cas9 protein are provided within a cell or are otherwise expressed within a cell and are combined together to form the Cas9 protein. This structure-guided design is essential since splitting Cas9 at ordered protein regions significantly impacts function. See Nishimasu, H. et al. Crystal structure of Cas9 in complex with guide RNA and target DNA. Cell 156, 935-949 (2014), Jinek, M. et al. Structures of Cas9 endonucleases reveal RNA-mediated conformational activation. Science 343, 1247997 (2014), Zetsche, B., Volz, S. E. & Zhang, F. A split-Cas9 architecture for inducible genome editing and transcription modulation. Nature biotechnology 33, 139-142 (2015), Nihongaki, Y., Kawano, F., Nakajima, T. & Sato, M. Photoactivatable CRISPR-Cas9 for optogenetic genome editing. Nature biotechnology 33, 755-760 (2015), Nishimasu, H. et al. Crystal Structure of Staphylococcus aureus Cas9. Cell 162, 1113-1126 (2015), Truong, D. J. et al. Development of an intein-mediated split-Cas9 system for gene therapy. Nucleic Acids Res 43, 6450-6458, doi:10.1093/nar/gkv601 (2015), and Fine, E. J. et al. Trans-spliced Cas9 allows cleavage of HBB and CCR5 genes in human cells using compact expression cassettes. Scientific reports 5, 10777 (2015).

According to one aspect, two portions of a Cas9 protein are provided within a cell or are otherwise expressed within a cell and are combined together to form the Cas9 protein. The two portions of the Cas9 protein are sufficient in length such that when they are combined into the Cas9 protein, the Cas9 protein has the function of co-localizing at a target nucleic acid with a guide RNA as described above. According to certain aspects, various methods known to those of skill in the art may be used to combine the two or more portions of a Cas9 protein together. Exemplary methods and linkers include split-intein protein trans-splicing for reconstituting the Cas9 protein as is known in the art and as described herein. Other methods include protein-protein interacting domains (see Zakeri, B., et al., Peptide tag forming a rapid covalent bond to a protein, through engineering a bacterial adhesion. Proc Natl Acad Sci USA, 2012. 109(12): p. E690-7 and Fierer, J. O., G. Veggiani, and M. Howarth, SpyLigase peptide-peptide ligation polymerizes affibodies to enhance magnetic cancer cell capture. Proc Natl Acad Sci USA, 2014. 111(13): p. E1176-81) or small molecule dependent interactions. See Los, G. V., et al., HaloTag: a novel protein labeling technology for cell imaging and protein analysis. ACS Chem Biol, 2008. 3(6): p. 373-82 and Keppler, A., et al., A general method for the covalent labeling of fusion proteins with small molecules in vivo. Nat Biotechnol, 2003. 21(1): p. 86-9; Mootz, H. D., Blum, E. S., Tyszkiewicz, A. B. & Muir, T. W. Conditional protein splicing: a new tool to control protein structure and function in vitro and in vivo. J Am Chem Soc 125, 10561-10569, doi:10.1021/ja0362813 (2003).

Embodiments of the present disclosure are directed to the use of a CRISPR/Cas system and, in particular, an enzymatically active Cas9 protein optionally having a functional group attached thereto, and one or more guide RNAs which includes a spacer sequence, a tracr mate sequence and a tracr sequence. According to certain aspects, a guide RNA which facilities enzymatic activity of the Cas9 protein has an exemplary spacer sequence including between 25 and 15 nucleotides in length. According to certain aspects, a guide RNA which inhibits enzymatic activity of the Cas9 protein has an exemplary spacer sequence including between 14 and 8 nucleotides in length. According to certain methods, two or more or a plurality of guide RNAs may be used in the practice of certain embodiments based on whether one of skill desires the species of enzymatically active Cas9 protein optionally having a functional group attached thereto to cut or nick a desired nucleic acid or to deliver the functional group to a desired nucleic acid so that the functional group can perform the function.

The term spacer sequence is understood by those of skill in the art and may include any polynucleotide having sufficient complementarity with a target nucleic acid sequence to hybridize with the target nucleic acid sequence and direct sequence-specific binding of a CRISPR complex to the target sequence. A CRISPR complex may include the guide RNA and the Cas9 protein. The guide RNA may be formed from a spacer sequence covalently connected to a tracr mate sequence (which may be referred to as a crRNA) and a separate tracr sequence, wherein the tracr mate sequence is hybridized to a portion of the tracr sequence. According to certain aspects, the tracr mate sequence and the tracr sequence are connected or linked such as by covalent bonds by a linker sequence, which construct may be referred to as a fusion of the tracr mate sequence and the tracr sequence. The linker sequence referred to herein is a sequence of nucleotides, referred to herein as a nucleic acid sequence, which connect the tracr mate sequence and the tracr sequence. Accordingly, a guide RNA may be a two component species (i.e., separate crRNA and tracr RNA which hybridize together) or a unimolecular species (i.e., a crRNA-tracr RNA fusion, often termed an sgRNA).

Tracr mate sequences and tracr sequences are known to those of skill in the art, such as those described in US 2014/0356958. The tracr mate sequence and tracr sequence used in the present disclosure is N20 or more to N8-gtatagagctagaaatagcaagttaaaataaggctagtccgttatcaacttgaaaaagtggcaccgagtcggtgc with N20-8 being the number of nucleotides complementary to a target locus of interest.

According to certain aspects, the guide RNA spacer sequence length determines whether the enzymatically active Cas9 optionally having a functional group attached thereto will function to cut or nick the target nucleic acid or to act as a nuclease null Cas9 and deliver the functional group if present to the target nucleic acid so that the functional group can perform the desired function. A guide RNA having a spacer sequence length where the enzymatically active Cas9 will cut or nick the target nucleic acid may be termed an “enzymatic guide RNA” to the extent that such a guide RNA facilitates enzymatic activity of the Cas9. An enzymatic guide RNA has an exemplary spacer sequence length of 25 to 15 nucleotides. A guide RNA having a spacer sequence length where the enzymatically active Cas9 will function as a nuclease null Cas9 and may be termed a “nonenzymatic guide RNA” to the extent that such a guide RNA will inhibit enzymatic activity of the Cas9. A nonenzymatic guide RNA has an exemplary spacer sequence length of 16 to 8 nucleotides. It is to be understood that the enzymatically active Cas9 may still be referred to as such even though it is used with a nonenzymatic guide RNA and where the enzymatically active Cas9 does not cut or nick the target nucleic acid. The enzymatically active Cas9 can be programmed to cut or operate as a nuclease null Cas9 based on the selected spacer sequence length. It is to be understood that for particular target nucleic acids, an exemplary enzymatic guide RNA length or an exemplary nonenzymatic guide RNA length may include 1 or two nucleotides outside of the exemplary ranges described herein.

According to certain aspects, the tracr mate sequence is between about 17 and about 27 nucleotides in length. According to certain aspects, the tracr sequence is between about 65 and about 75 nucleotides in length. According to certain aspects, the linker nucleic acid sequence is between about 4 and about 6.

The functional group or function conferring protein or domain may be joined, fused, connected, linked or otherwise tethered such as by covalent hoods to the enzymatically active Cas9 protein using methods known to those of skill in the art.

Functional groups or function conferring proteins or domains within the scope of the present disclosure include transcriptional modulators or effector domains known to those of skill in the art. Suitable transcriptional modulators include transcriptional activators. According to one aspect, the transcriptional regulator protein or domain upregulates expression of the target nucleic acid. Suitable transcriptional modulators include transcriptional repressors. According to one aspect, the transcriptional regulator protein or domain downregulates expression of the target nucleic acid. Exemplary transcriptional activators include VP64, VP16, VP160, VP48, VP96, p65, Rta, VPR, hsf1, and p300 Suitable transcriptional repressors include KRAB. Transcriptional activators and transcriptional repressors can be readily identified by one of skill in the art based on the present disclosure

Functional groups, function conferring proteins or domains within the scope of the present disclosure include detectable groups or markers or labels. Such detectable groups or markers or labels can be detected or imaged using methods known to those of still in the art to identify the location of the target nucleic acid sequence. Indirect attachment of a detectable label or maker is contemplated by aspects of the present disclosure. Detectable labels or markers can be readily identified by one of skill in the art based on the present disclosure. Detectable groups include fluorescent proteins such as GFP, RFP, BFP, EYFP, sfGFP, mcherry, iRFP, citrine, morange, cerulean, mturquoise, EBFP, EBFP2, Azurite, mKalama1, ECP, CYPET, mturquoise2, YFP, Venus, and Ypet and the like. Other useful detectable groups include spytag, spycatcher, snap tags, biotin, streptavidin, and suntag and the like.

Functional groups, function conferring proteins or domains within the scope of the present disclosure include binding functional groups which may function to bind to desired molecules. Such binding functional groups include aptamers ms2 to MCP, pp7 to PCP, com to Com binding protein, inteins, FKBP to FRB, pMAG to nMAG and Cry2 and the like.

According to one aspect, embodiments described herein include guide RNA having a length including the sum of the lengths of a spacer sequence, tracr mate sequence, tracr sequence, and linker sequence (if present). Accordingly, such a guide RNA may be described by its total length which is a sum of its spacer sequence, tracr mate sequence, tracr sequence, and linker sequence. According to this aspect, all of the ranges for the spacer sequence, tracr mate sequence, tracr sequence, and linker sequence (if present) are incorporated herein by reference and need not be repeated. One of skill will readily be able to sum each of the portions of a guide RNA to obtain the total length of the guide RNA sequence. Aspects of the present disclosure are directed to methods of making such guide RNAs as described herein by expressing constructs encoding such guide RNA using promoters and terminators and optionally other genetic elements as described herein.

According to certain aspects, the guide RNA and the enzymatically active Cas9 optionally having a functional group attached thereto which interacts with the guide RNA are foreign to the cell into which they are introduced or otherwise provided. According to this aspect, the guide RNA and the enzymatically active Cas9 optionally having a functional group attached thereto are nonnaturally occurring in the cell in which they are introduced, or otherwise provided. To this extent, cells may be genetically engineered or genetically modified to include the CRISPR/Cas systems described herein.

One such CRISPR/Cas system uses the S. pyogenes Cas9 nuclease (Sp. Cas9), an extremely high-affinity (see Sternberg, S. H., Redding, S., Jinek, M., Greene, E. C. & Doudna, J. A. DNA interrogation by the CRISPR RNA-guided endonuclease Cas9. Nature 507, 62-67 (2014) hereby incorporated by reference in its entirety), programmable DNA-binding protein isolated from a type II CRISPR-associated system (see Garneau, J. E. et al. The CRISPR/Cas bacterial immune system cleaves bacteriophage and plasmid DNA. Nature 468, 67-71 (2010) and Jinek, M. et al. A programmable dual-RNA-guided DNA endonuclease in adaptive bacterial immunity. Science 337, 816-821 (2012) each of which are hereby incorporated by reference in its entirety). The DNA locus targeted by Cas9 precedes a three nucleotide (nt) 5′-NGG-3′ “PAM” sequence, and matches a 15-22-nt guide or spacer sequence within a Cas9-bound RNA cofactor, referred to herein and in the art as a guide RNA. Altering this guide RNA is sufficient to target Cas9 to a target nucleic acid. In a multitude of CRISPR-based biotechnology applications, the guide is often presented in a so-called sgRNA (single guide RNA), wherein the two natural Cas9 RNA cofactors (crRNA and tracrRNA) are fused via an engineered loop.

Embodiments of the present disclosure are directed to a method of delivering a functional group or moiety attached to a enzymatically active Cas9 protein to a target nucleic acid in a cell comprising providing to the cell the enzymatically active Cas9 protein having the functional group or moiety attached thereto and a guide RNA having spacer sequence between 16 and 8 nucleotides in length wherein the guide RNA and the Cas9 protein form a co-localization complex with the target nucleic acid and where the enzymatically active Cas9 protein is rendered non-endonucleolytically active and where the functional group or moiety is delivered to the target nucleic acid. Methods described herein can be performed in vitro, in vivo or ex vivo. According to one aspect, the cell is a eukaryotic cell or a prokaryotic cell. According to one aspect, the cell is a bacteria cell, a yeast cell, a mammalian cell, a plant cell or an animal cell. According to one aspect, the Cas9 protein is an enzymatically active Cas9 protein, a Cas9 protein wild-type protein, or an enzymatically active Cas9 nickase. Additional exemplary Cas9 proteins include Cas9 proteins attached to, bound to or fused with functional proteins such as transcriptional regulators, such as transcriptional activators or repressors VPR, a Fok-domain, such a Fok 1, an aptamer, a binding protein, PP7, MS2 and the like.

Embodiments of the present disclosure are directed to a method of delivering an enzymatically active Cas9 protein optionally having a functional group attached thereto to cells within a subject comprising administering to the subject, such as systemically administering to the subject, such as by intravenous administration or injection, intraperitoneal administration or injection, intramuscular administration or injection, intracranial administration or injection, intraocular administration or injection, subcutaneous administration or injection, a enzymatically active Cas9 protein optionally having a functional group attached thereto or a nucleic acid encoding the enzymatically active Cas9 protein optionally having a functional group attached thereto.

Embodiments of the present disclosure are directed to a method of delivering a guide RNA to cells within a subject comprising administering to the subject, such as systemically administering to the subject, such as by intravenous administration or injection, intraperitoneal administration or injection, intramuscular administration or injection, intracranial administration or injection, intraocular administration or injection, subcutaneous administration or injection, a guide RNA or a nucleic acid encoding the guide RNA.

Embodiments of the present disclosure are directed to a method of delivering an enzymatically active Cas9 protein optionally having a functional group attached thereto and a guide RNA to cells within a subject comprising administering to the subject, such as systemically administering to the subject, such as by intravenous administration or injection, intraperitoneal administration or injection, intramuscular administration or injection, intracranial administration or injection, intraocular administration or injection, subcutaneous administration or injection, an enzymatically active Cas9 optionally having a functional group attached thereto or a nucleic acid encoding the enzymatically active Cas9 protein optionally having a functional group attached thereto and a guide RNA or a nucleic acid encoding the guide RNA.

Methods of non-viral delivery of nucleic acids or native DNA binding protein, native guide RNA or other native species include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term native includes the protein, enzyme or guide RNA species itself and not the nucleic acid encoding the species.

Regulatory elements are contemplated for use with the methods and constructs described herein. The term “regulatory element” is intended to include promoters, enhancers, internal ribosomal entry sites (IRES), and other expression control elements (e.g. transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. Regulatory elements may also direct expression in an inducible manner, such as in a small-molecule dependent or light-dependent manner. In some embodiments, a vector may comprise one or more pol III promoter (e.g. 1, 2, 3, 4, 5, or more pol III promoters), one or more pol II promoters (e.g. 1, 2, 3, 4, 5, or more pol II promoters), one or more pol I promoters (e.g. 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see, e.g., Boshart et al, Cell, 41:521-530 (1985)), the SV40 promoter, the dihydrofolate reductase promoter, the β-actin promoter, the phosphoglycerol kinase (PGK) promoter, and the EF1α promoter and Pol II promoters described herein. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (Proc. Natl. Acad. Sci. USA., Vol. 78(3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., clustered regularly interspersed short palindromic repeats (CRISPR) transcripts, proteins, enzymes, mutant forms thereof, fusion proteins thereof, etc.).

Aspects of the methods described herein may make use of terminator sequences. A terminator sequence includes a section of nucleic acid sequence that marks the end of a gene or operon in genomic DNA during transcription. This sequence mediates transcriptional termination by providing signals in the newly synthesized mRNA that trigger processes which release the mRNA from the transcriptional complex. These processes include the direct interaction of the mRNA secondary structure with the complex and/or the indirect activities of recruited termination factors. Release of the transcriptional complex frees RNA polymerase and related transcriptional machinery to begin transcription of new mRNAs. Terminator sequences include those known in the art and identified and described herein.

Aspects of the methods described herein may make use of epitope tags and reporter gene sequences. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, betaglucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

The following examples are set forth as being representative of the present disclosure. These examples are not to be construed as limiting the scope of the present disclosure as these and other equivalent embodiments will be apparent in view of the present disclosure, figures and accompanying claims.

### Examples

CRISPR-Cas9 holds tremendous promise in correcting genetic defects, and its delivery by adeno-associated viruses (AAVs) is thought to be exceptionally safe. However, immunological reactions against encoded transgenes and/or the viral capsid have sometimes been observed (reviewed in Mays, L. E. & Wilson, J. M. The complex and evolving story of T cell activation to AAV vector-encoded transgene products. Mol Ther 19, 16-27, doi:10.1038/mt.2010.250 (2011)). Hence, in this study, it has been sought to first establish a flexible AAV-CRISPR-Cas9 platform that enables the wide spectrum of unrealized applications in vivo. Second, the host response to the system has been tracked. This is important because the exogenous nature of AAV-CRISPR-Cas9 might incite detrimental host reactions against the encoded transgenes and/or viral capsid (reviewed in Mays, L. E. & Wilson, J. M. The complex and evolving story of T cell activation to AAV vector-encoded transgene products. Mol Ther 19, 16-27, doi:10.1038/mt.2010.250 (2011)). Understanding the host responses towards AAV-CRISPR-Cas9 would identify confounding factors that impact experimental rigor, highlight relevant considerations for clinical translation, and provide a roadmap for engineering efficient genome manipulation systems. Specifically, this example describes the immunogenicity of AAV-CRISPR-Cas9 in mice, specifically that of AAV-split-Cas9, a platform capable of postnatal genome-editing, transcriptional regulation, and further domain fusions. AAV elicits a humoral immune response, inducing antibodies targeting motifs associated with viral functions. Cas9 elicits both humoral and cellular immune responses, but its delivery by AAV mitigates overt tissue or cellular damage seen with alternative delivery methods. This study provides the first demonstration of postnatal CRISPR-Cas9 applications beyond genome-editing, and elucidates the AAV-CRISPR-Cas9 safety profile necessary for bringing it into the clinic.

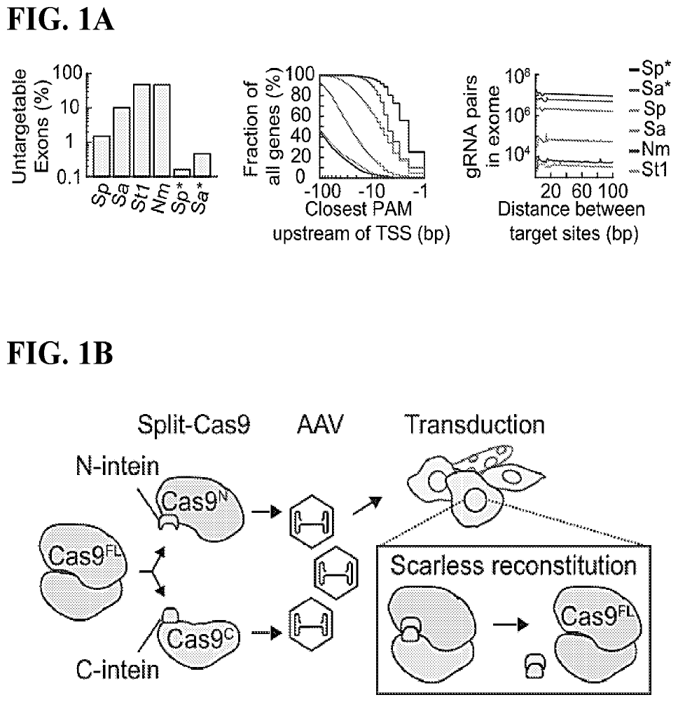

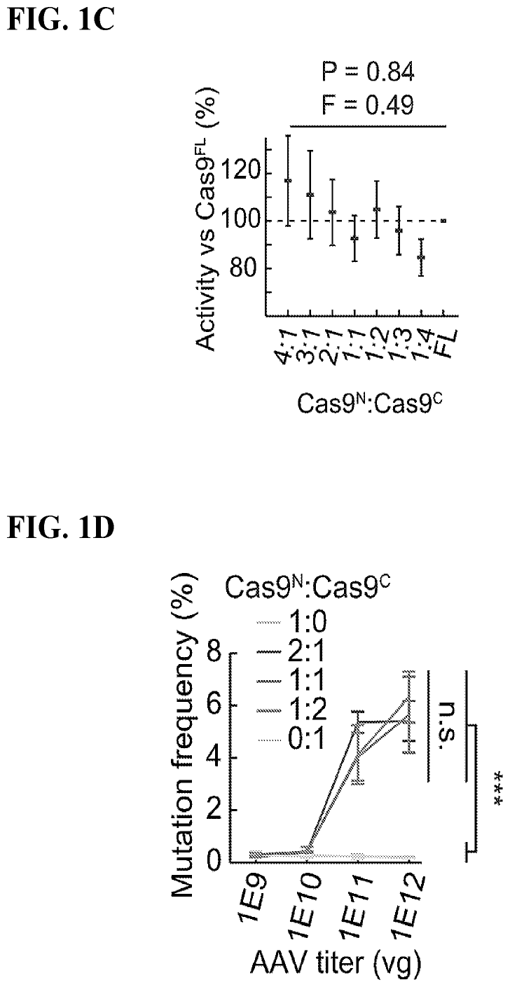

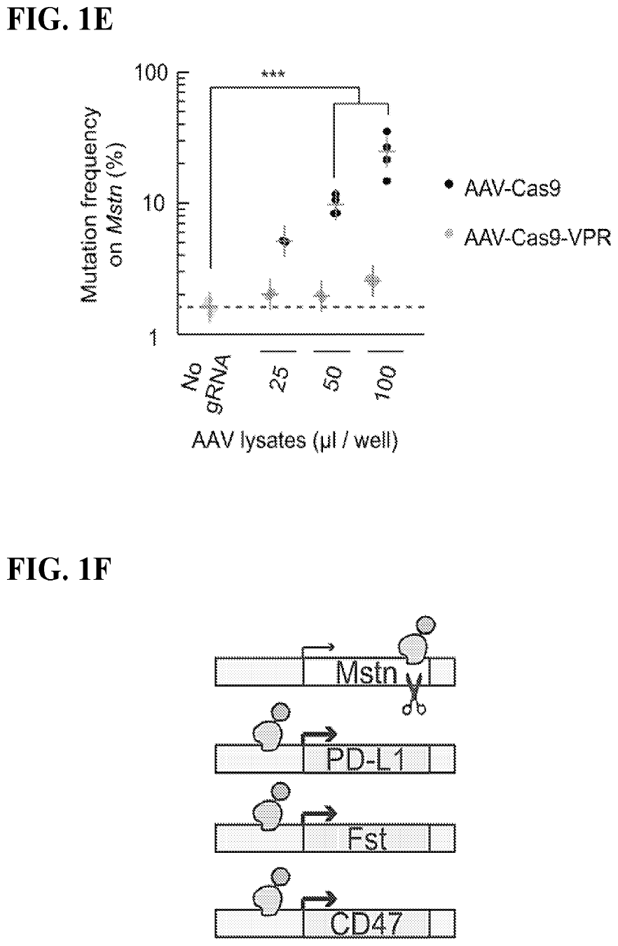

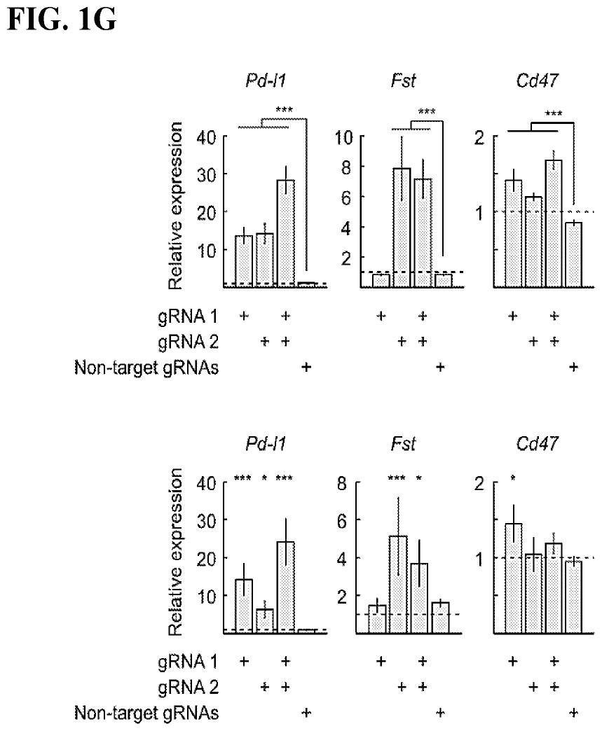

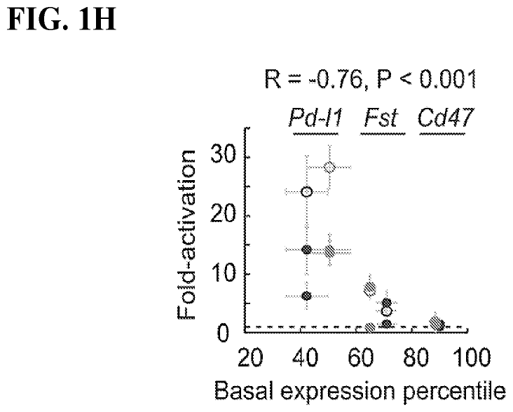

Of the various CRISPR-Cas9 (Mali, P. et al. RNA-guided human genome engineering via Cas9. Science 339, 823-826, doi:10.1126/science.1232033 (2013); Cong, L. et al. Multiplex genome engineering using CRISPR/Cas systems. Science 339, 819-823, doi:10.1126/science.1231143 (2013); Jinek, M. et al. A programmable dual-RNA-guided DNA endonuclease in adaptive bacterial immunity. Science 337, 816-821, doi:10.1126/science.1225829 (2012); Ran, F. A. et al. In vivo genome editing using Staphylococcus aureus Cas9. Nature 520, 186-191, doi:10.1038/nature14299 (2015); Esvelt, K. M. et al. Orthogonal Cas9 proteins for RNA-guided gene regulation and editing. Nature methods 10, 1116-1121, doi:10.1038/nmeth.2681 (2013)) and recently characterized CRISPR-Cpf1 (Zetsche, B. et al. Cpf1 is a single RNA-guided endonuclease of a class 2 CRISPR-Cas system. Cell 163, 759-771, doi:10.1016/j.cell.2015.09.038 (2015)) orthologs, this example chose to examine the Streptococcus pyogenes Cas9 (SpCas9) due to multiple attractive features. SpCas9 has the least restrictive protospacer adjacent motif (PAM) requirement, which fundamentally dictates the density of possible target sites per given genome (FIG. 1A). Conversely, more restrictive PAM requirements (e.g., St1 (Esvelt, K. M. et al. Orthogonal Cas9 proteins for RNA-guided gene regulation and editing. Nature methods 10, 1116-1121, doi:10.1038/nmeth.2681 (2013)); Nm (Esvelt, K. M. et al. Orthogonal Cas9 proteins for RNA-guided gene regulation and editing. Nature methods 10, 1116-1121, doi:10.1038/nmeth.2681 (2013)); and Sa (Ran, F. A. et al. In vivo genome editing using Staphylococcus aureus Cas9. Nature 520, 186-191, doi:10.1038/nature14299 (2015)) Cas9s; As and Lb Cpf1s (Zetsche, B. et al. Cpf1 is a single RNA-guided endonuclease of a class 2 CRISPR-Cas system. Cell 163, 759-771, doi:10.1016/j.cell.2015.09.038 (2015)) render significant numbers of target sites inaccessible Combining SpCas9 with AAV is hence highly enticing to access the broadest targeting range in vivo. Furthermore, the array of CRISPR-Cas9 applications beyond genome-editing has yet to be realized in vivo, in part due to the large sizes of Cas9 transgenes that leave little space for additional function-conferring elements within current AAV designs (Ran. F A et al. In vivo genome editing using Staphylococcus aureus Cas9. Nature 520, 186-191, doi:10.1038/nature14299 (2015); Nelson, C. E. et al. In vivo genome editing improves muscle function in a mouse model of Duchenne muscular dystrophy. Science 351, 403-407, doi:10.1126/science.aad5143 (2016); Tabebordbar, M. et al. In vivo gene editing in dystrophic mouse muscle and muscle stem cells. Science 351, 407-411, doi:10.1126/science.aad5177 (2016); Long, C. et al. Postnatal genome editing partially restores dystrophin expression in a mouse model of muscular dystrophy. Science 351, 400-403, doi:10.1126/science.aad5725 (2016); Yang, Y. et al. A dual AAV system enables the Cas9-mediated correction of a metabolic liver disease in newborn mice. Nature biotechnology, doi:10.1038/nbt.3469 (2016); Yin, H. et al. Therapeutic genome editing by combined viral and non-viral delivery of CRISPR system components in vivo. Nature biotechnology, doi:10.1038/nbt.3471 (2016); Senis, E. et al. CRISPR/Cas9-mediated genome engineering: an adeno-associated viral (AAV) vector toolbox. Biotechnology journal 9, 1402-1412, doi:10.1002/biot.201400046 (2014); Swiech, L. et al. In vivo interrogation of gene function in the mammalian brain using CRISPR-Cas9. Nature biotechnology 33, 102-106. doi: 10.1038/nbt.3055 (2015)) (pay load limit ≤4.7 kb). This obstacle is exacerbated with the most widely used, but larger SpCas9 (4.2 kb), which makes packaging of even the minimum functional cassette challenging (Long. C. et. al. Postnatal genome editing partially restores dystrophin expression in a mouse model of muscular dystrophy. Science 351, 400-403, doi:10.1126/science.aad5725 (2016); Senis, E. et al. CRISPR/Cas9-mediated genome engineering: an adeno-associated viral (AAV) vector toolbox. Biotechnology journal 9, 1402-1412, doi:10.1002/biot.201400046 (2014); Swiech, L. et al. In vivo interrogation of gene function in the mammalian brain using CRISPR-Cas9. Nature biotechnology 33, 102-106, doi:10.1038/nbt.3055 (2015)). Resolving this challenge would enable novel applications currently unachievable.

Hence, in this example, it is sought to firstly, combine AAV, SpCas9, and fusion domains for a platform capable of postnatal genome-editing and epigenetic modulation, and secondly, to investigate the immunogenicity of the therapeutic modality.

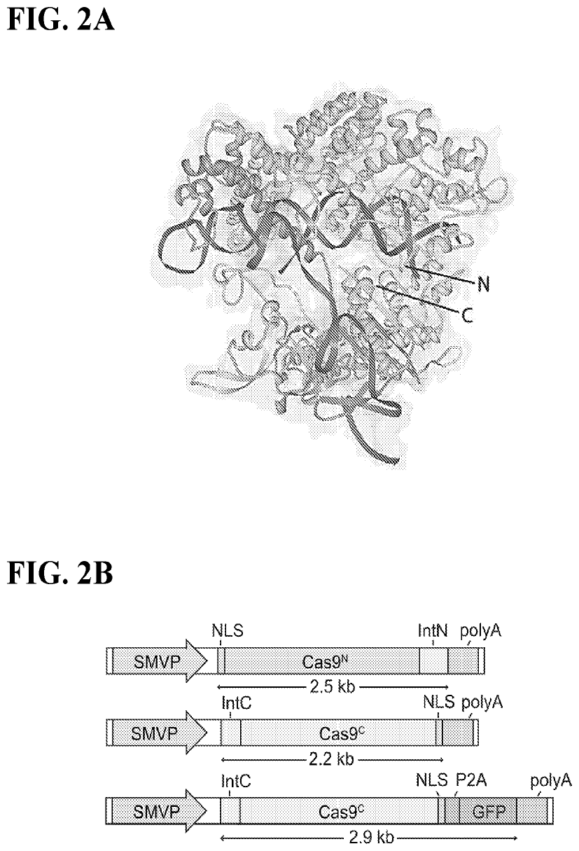

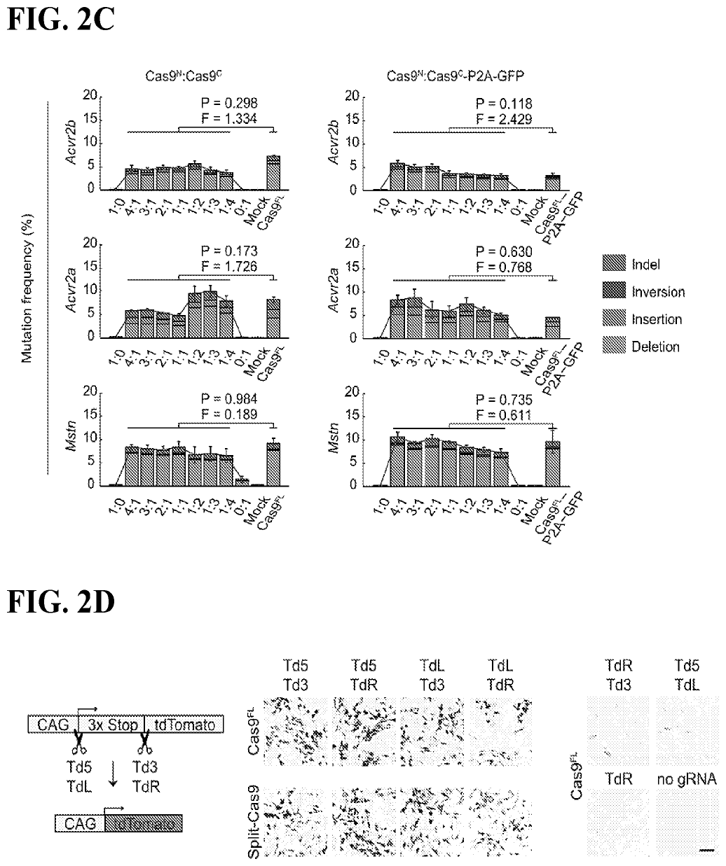

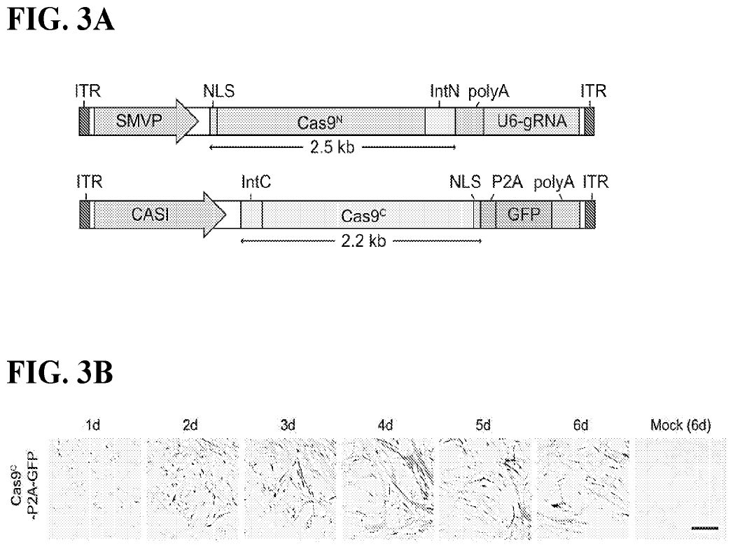

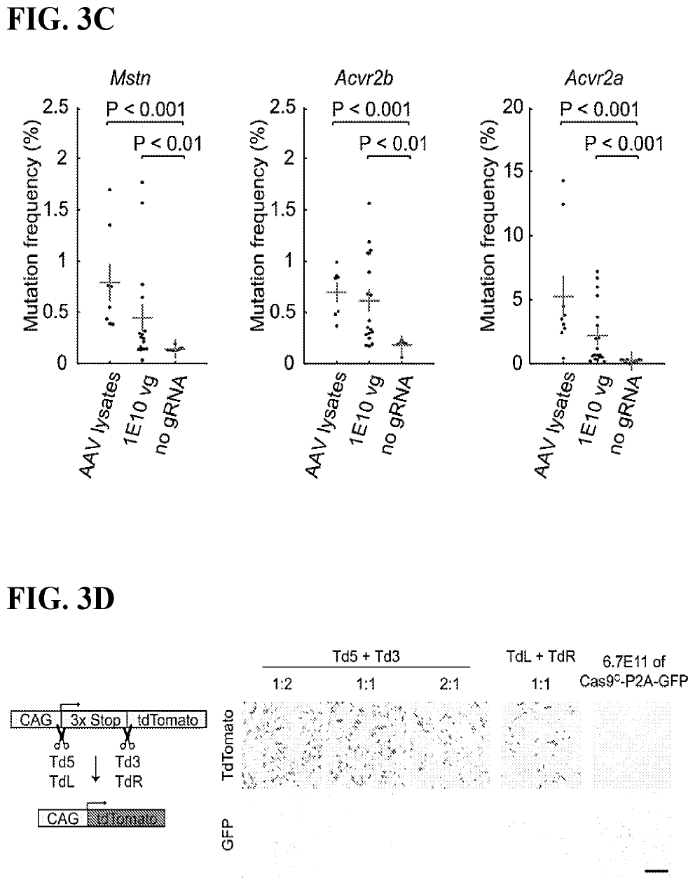

To date, determined structures of Cas9 and Cpf1 proteins generally adopt a bi-lobed architecture (SpCas9 (Nishimasu, H. et al. Crystal structure of Cas9 in complex with guide RNA and target DNA. Cell 156, 935-949, doi:10.1016/j.cell.2014.02.001 (2014); Jinek, M. et al. Structures of Cas9 endonucleases reveal RNA-mediated conformational activation. Science 343, 1247997, doi:10.1126/science.1247997 (2014)), SaCas9 (Nishimasu, H. et al. Crystal Structure of Staphylococcus aureus Cas9. Cell 162, 1113-1126, doi:10.1016/j.cell.2015.08.007 (2015)), AnaCas9 (Jinek, M. et al. Structures of Cas9 endonucleases reveal RNA-mediated conformational activation. Science 343, 1247997, doi:10.1126/science.1247997 (2014)), LbCpf1 (Dong D, Ren K, Qiu X, Zheng J, Guo M, Guan X, Liu H, Li N, Zhang B, Yang D, Ma C, Wang S, Wu D, Ma Y, Fan S, Wang J, Gao N, Huang Z, The crystal structure of Cpf1 in complex with CRISPR RNA, Nature, 2016 April 28; 532(7600):522-6) AsCpf1 (Yamano T, Nishimasu H, Zetsche B, Hirano H, Slaymaker I M, Li Y, Fedorova I, Nakane T, Makarova K S, Koonin E V, Ishitani R, Zhang F, Nureki O, Crystal Structure of Cpf1 in Complex with Guide RNA and Target DNA, Cell, 2016 May 5; 165(4):949-62), but not FnCas9 (Hirano, H. et al. Structure and Engineering of Francisella novicida Cas9. Cell 164. 950-961. doi:10.1016/j.cell.2016.01.039 (2016))), offering the clue that they can be split into two well-folded halves. Indeed, previous reports have demonstrated the feasibility of split-Cas9, albeit all in cell cultures and with different design principles (Nishimasu, H. et al. Crystal Structure of Staphylococcus aureus Cas9. Cell 162, 1113-1126, doi:10.1016/j.cell.2015.08.007 (2015); Zetsche, B., Volz, S. E. & Zhang, F. A split-Cas9 architecture for inducible genome editing and transcription modulation. Nature biotechnology 33, 139-142, doi:10.1038/nbt.3149 (2015); Wright, A. V. et al. Rational design of a split-Cas9 enzyme complex. Proceedings of the National Academy of Sciences of the United States of America 112, 2984-2989, doi:10.1073/pnas.1501698112 (2015); Nihongaki, Y., Kawano, F., Nakajima, T. & Sato, M. Photoactivatable CRISPR-Cas9 for optogenetic genome editing. Nature biotechnology 33, 755-760, doi:10.1038/nbt.3245 (2015); Truong, D. J. et al. Development of an intein-mediated split-Cas9 system for gene therapy. Nucleic Acids Res 43, 6450-6458, doi:10.1093/nar/gkv601 (2015); Fine, E. J. et al. Trans-spliced Cas9 allows cleavage of HBB and CCR5 genes in human cells using compact expression cassettes. Scientific reports 5, 10777, doi:10.1038/srep10777 (2015)). This example describes as an exemplary embodiment where SpCas9 is specifically split at its disordered linker between amino-acid residues V713 and D718, hypothesizing that this maintains protein folding for each lobe, such that the full-length Cas9 (Cas9^{FL}) can be reconstituted seamlessly in vivo by split-intein protein trans-splicing (Li, J., Sun, W., Wang. B., Xiao, X. & Liu, X. Q. Protein trans-splicing as a means for viral vector-mediated in vivo gene therapy. Hum Gene Ther 19, 958-964, doi:10.1089/hum.2008.009 (2008)) (FIG. 1B). Thus, the Cas9 N-terminal lobe is fused with the Rhodothermus marinus N-split-intein (Cas9N) (2.5 kb), and the C-terminal lobe is fused with C-split-intein (Cas9c) (2.2 kb) (FIGS. 2A and 2B), and each is designed to be individually packaged into a separate AAV vector (FIG. 3A), thereby liberating >2 kb within each AAV vector for additional elements. In transfected cells, split-Cas9 was fully active, targeting all endogenous genes tested at efficiencies 85% to 115% of Cas9^{FL} (FIG. 1C and FIGS. 2C and 2D). Full activity from structure-guided split-intein reconstitution (Truong, D. J. et al. Development of an intein-mediated split-Cas9 system for gene therapy. Nucleic Acids Res 43, 6450-6458, doi:10.1093/nar/gkv601 (2015)) contrasts with sub-optimal activity from non-covalent heterodimerization (Nishimasu, H. et al. Crystal Structure of Staphylococcus aureus Cas9. Cell 162, 1113-1126, doi:10.1016/j.cell.2015.08.007 (2015); Zetsche, B., Volz, S. E. & Zhang, F. A split-Cas9 architecture for inducible genome editing and transcription modulation. Nature biotechnology 33, 139-142, doi:10.1038/nbt.3149 (2015); Wright, A. V. et al. Rational design of a split-Cas9 enzyme complex. Proceedings of the National Academy of Sciences of the United States of America 112, 2984-2989, doi:10.1073/pnas.1501698112 (2015); Nihongaki, Y., Kawano, F., Nakajima, T. & Sato, M. Photoactivatable CRISPR-Cas9 for optogenetic genome editing. Nature biotechnology 33, 755-760, doi:10.1038/nbt.3245 (2015)), suggesting that scarless protein ligation preserves Cas9 structure and function. Next, Cas9^{C}-P2A-turboGFP and Cas9N-U6-gRNAs were packaged into AAV serotype DJ (AAV-Cas9-gRNAs) (FIG. 3A) and the viruses were to cultured cells. AAV-Cas9-gRNAs modified all targeted genes in differentiated myotubes (FIG. 1D, FIGS. 3B and 3C), tail-tip fibroblasts (FIG. 3D) and spermatogonial cells (FIG. 1E), demonstrating robustness in three distinct cell types of proliferative and terminally differentiated states. Therefore, split-Cas9 retains full activity of Cas9^{FL} and opens the spectrum of AAV-CRISPR-Cas9 applications previously unattainable, such as the programmable targeting of fusion domains towards DNA.

CRISPR-Cas9-mediated epigenetic regulation has not been demonstrated in mice, but this ability would address a whole spectrum of human epigenetic diseases irresolvable by genome-editing. Hence the next experiment capitalized on the extra viral capacity of AAV-split-Cas9 to incorporate transcription-activator fusion domains (1.6 kb tripartite VPR (Chavez, A. et al. Highly efficient Cas9-mediated transcriptional programming. Nature methods 12, 326-328, doi:10.1038/nmeth.3312 (2015))) for gene expression upregulation (AAV-Cas9-VPR). This experiment further harnessed the findings that nuclease-active Cas9 can be programmed with truncated gRNAs to bind genomic loci without inducing DNA breaks, thereby allowing a single Cas9 fusion protein to simultaneously effect genome-editing and epigenetic regulation (Kiani, S. et al. Cas9 gRNA engineering for genome editing, activation and repression. Nature methods 12, 1051-1054, doi:10.1038/nmeth.3580 (2015); Dahlman, J. E. et al. Orthogonal gene knockout and activation with a catalytically active Cas9 nuclease. Nature biotechnology 33, 1159-1161, doi:10.1038/nbt.3390 (2015)) (FIG. 1F). Following transduction, nuclease-active AAV-Cas9-VPR programmed with truncated gRNAs (14-15 nt spacers) upregulated gene expression of the targeted PD-L1, FST, and CD47 genes (FIG. 1G) As with nuclease-inactive ‘dead’ Cas9^{FL} (dCas9)-activators (Chavez. A. et al. Highly efficient Cas9-mediated transcriptional programming Nature methods 12. 326-328. doi 10.1038/nmeth.3312 (2015); Konermann, S. et al. Genome-scale transcriptional activation by an engineered CRISPR-Cas9 complex. Nature 517. 583-588, doi:10.1038/nature14136 (2015)), gene activation by AAV-Cas9-VPR inversely correlated with the basal expression levels of the target genes (FIG. 1H). It is noted that when programmed with full-length gRNAs, AAV-Cas9-VPR does not retain full endonucleolytic function of AAV-Cas9 (FIG. 1E) Hence, split-Cas9 enables fusion of function-conferring domains within AAV₈, and untethered split-Cas9 offers more robust DNA-cleavage. Importantly, AAV-Cas9^{FL} design as previously described are unable to accommodate the 16 kb VPR domain fusion.

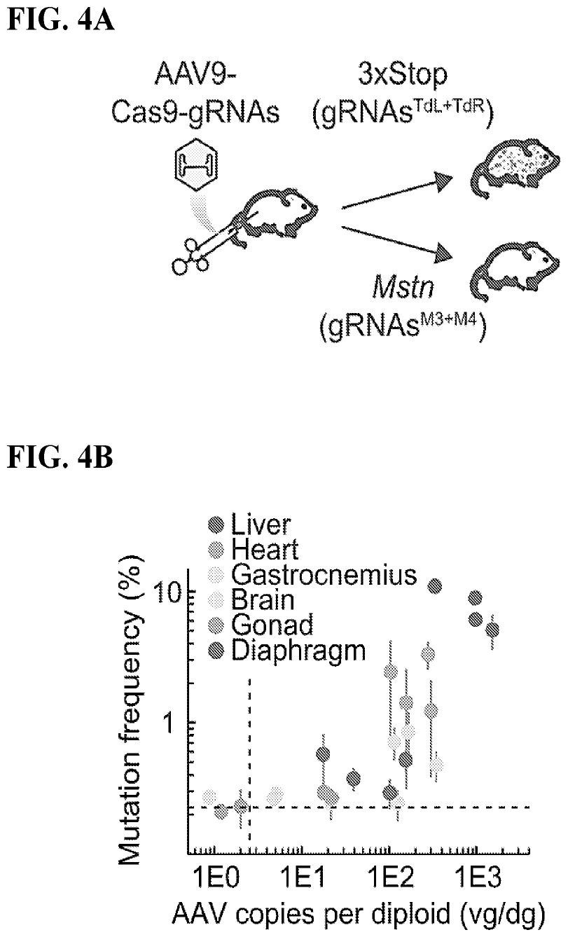

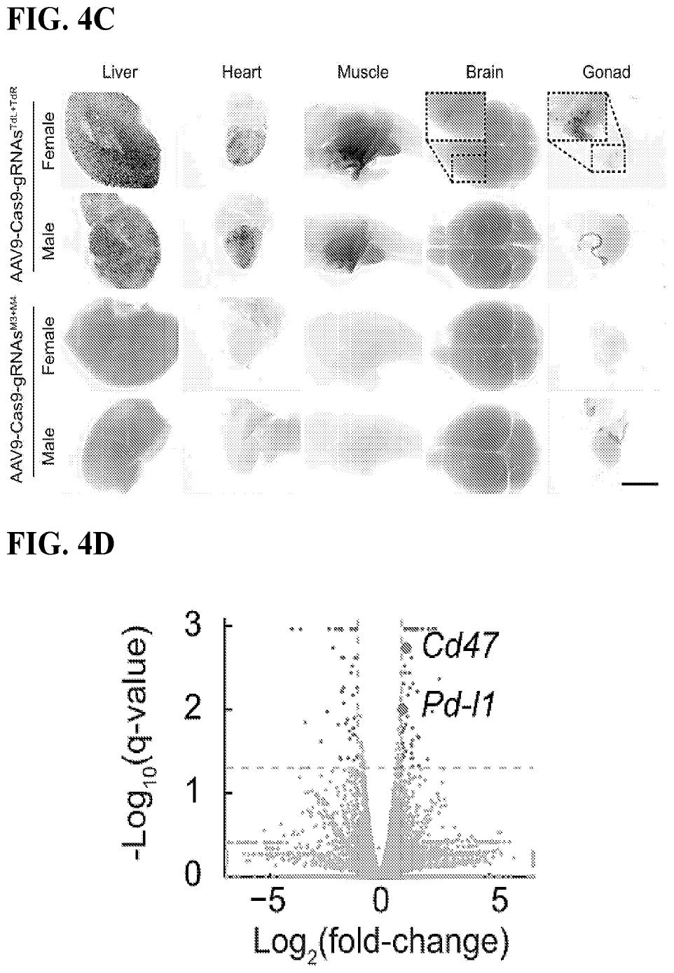

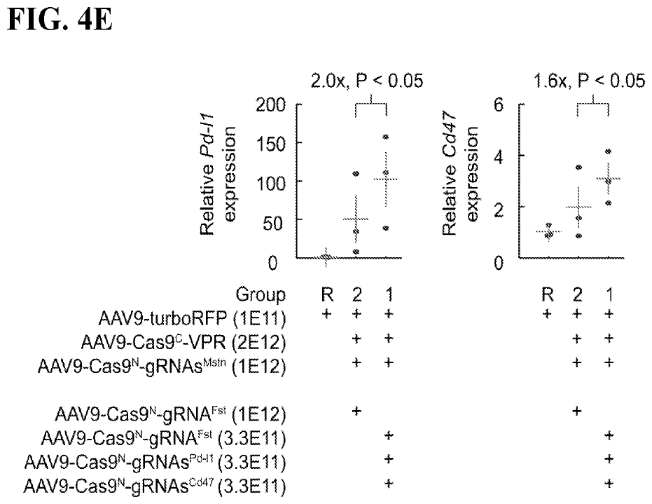

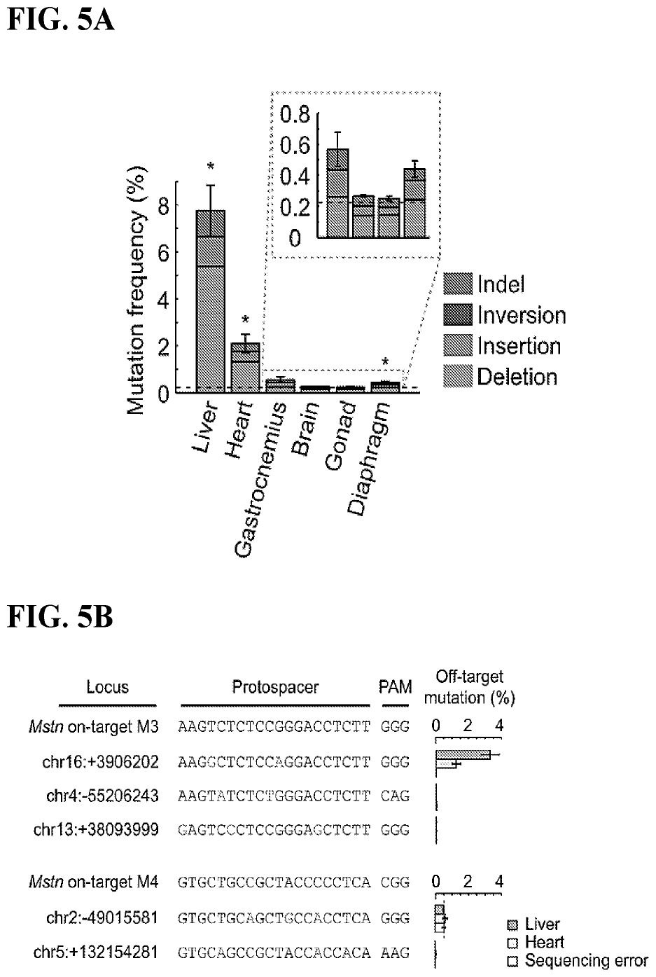

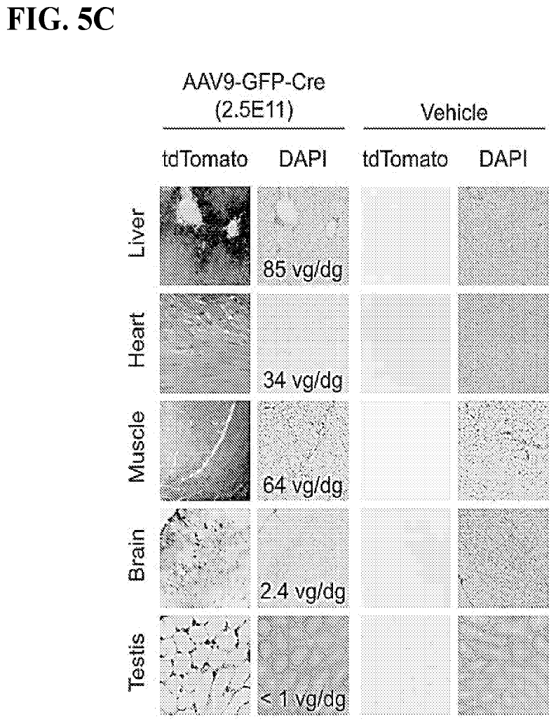

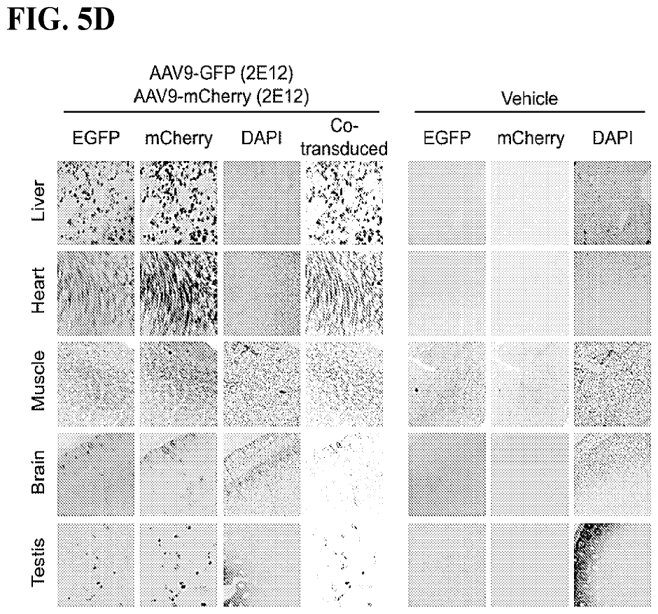

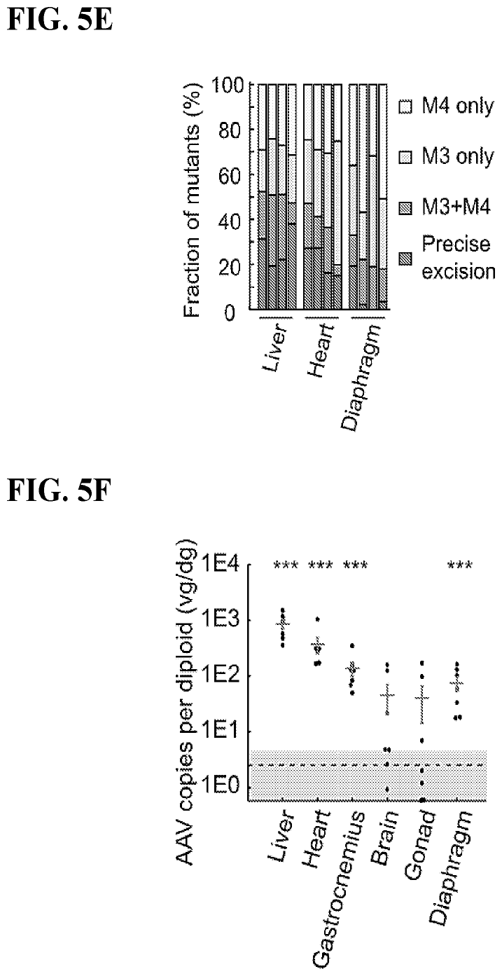

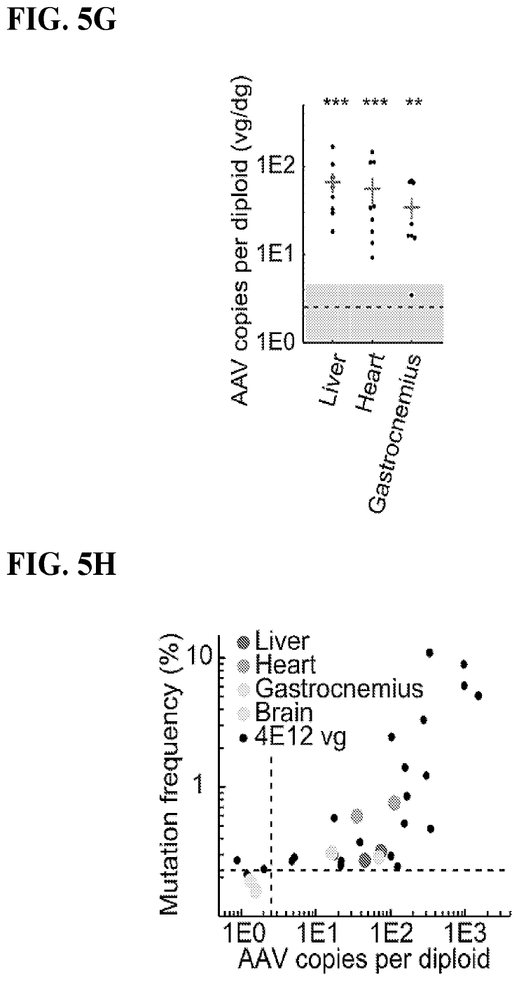

To demonstrate functional in vivo, AAV-Cas9-gRNAs targeting Msin was next pseudotyped to serotype 9 (AAV9-Cas9-gRNAs^{M3→M4}) and systematically delivered the viruses (5E11 or 4E12 vector genomes, vg) into neonatal mice by intraperitoneal injection (FIG. 4A). All AAV experiments in vivo were conducted in a randomized and double-blind fashion. Deep-sequencing of whole tissues from injected mice revealed a range of editing frequencies (up to 10.9%), similar to those observed in cell culture (plasmid, up to 10.7%. AAV, up to 6.3-24.6%). It was observed that editing frequencies exhibited inter-tissue bias for both on-target (Mstn) and off-target (chr6-3906202) sites (FIGS. 5A and 5B), which suggests either absence of transduction m some cells, or that most cells were transduced but at different efficiencies. Infection assays argue against the first scenario, because the AAV9 dosage used was in excess to transduce all examined cells within the liver, heart, and skeletal muscle (FIG. 5C). Moreover, dual AAV9s co-transduced across most cells (FIG. 5D), suggesting that gRNAs and split-Cas9 co-deliver, and further supports the feasibility of applications involving multiple gRNAs (FIG. 5E) and Cas9 proteins. The second scenario was then tested by measuring viral concentration per cell, using qPCR to quantify AAV vector genomes (vg) per mouse diploid genome (dg). Consistent with prior findings (Zincarelli, C., Soltys, S., Rengo, G. & Rabinowitz, J. E. Analysis of AAV serotypes 1-9 mediated gene expression and tropism in mice after systemic injection. Mol Ther 16, 1073-1080, doi:10.1038/mt.2008.76 (2008)), AAV9 showed preferential tropism for liver, heart and skeletal muscle (vg/dg of 850, 370, 140, respectively), while lower vg/dg were detected in the brain and gonads (FIG. 5F). Strikingly, gene-editing frequencies correlated strongly with AAV vector copies (FIG. 4B, FIGS. 5G and 5H), indicating that delivery efficiency dictates mutation rate. Therefore, despite sufficiency in infecting most cells, higher AAV9-Cas9-gRNA copies per cell continue to increase editing rates.

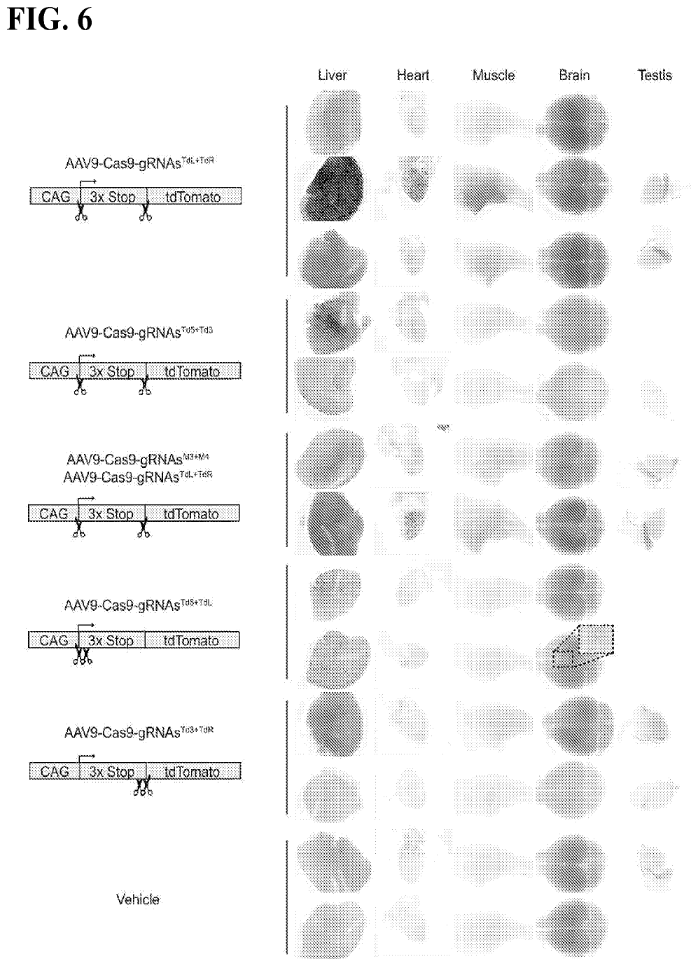

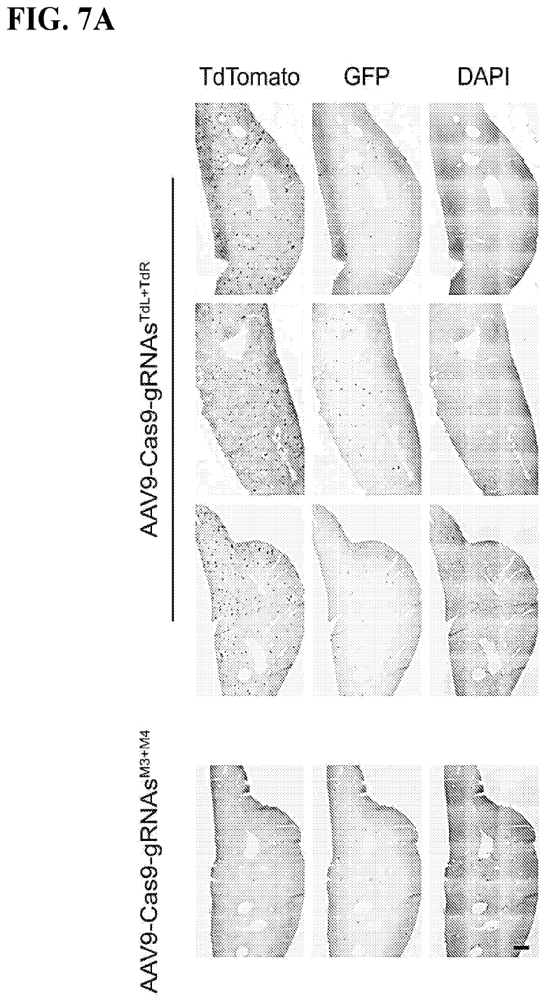

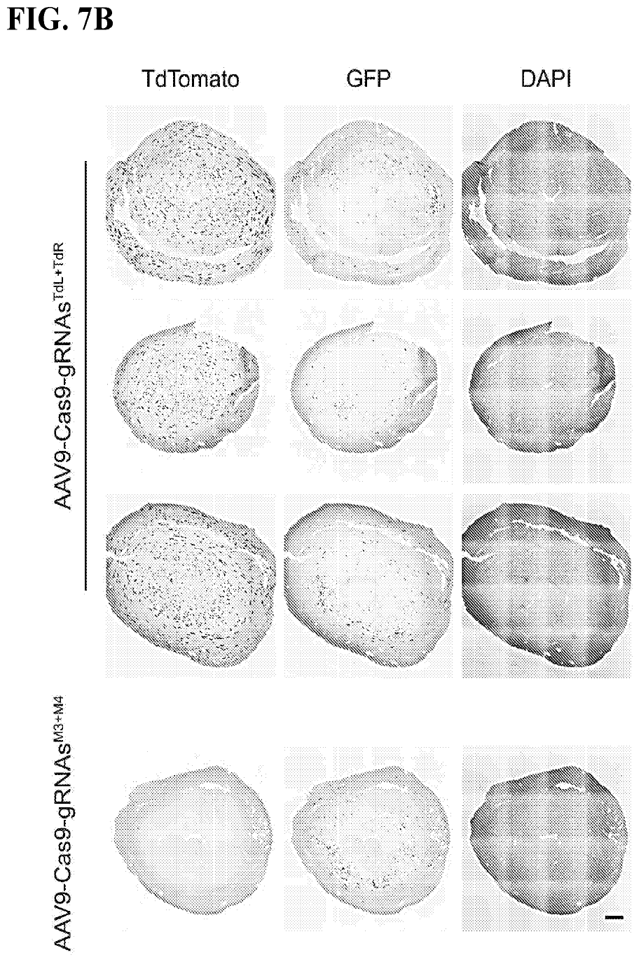

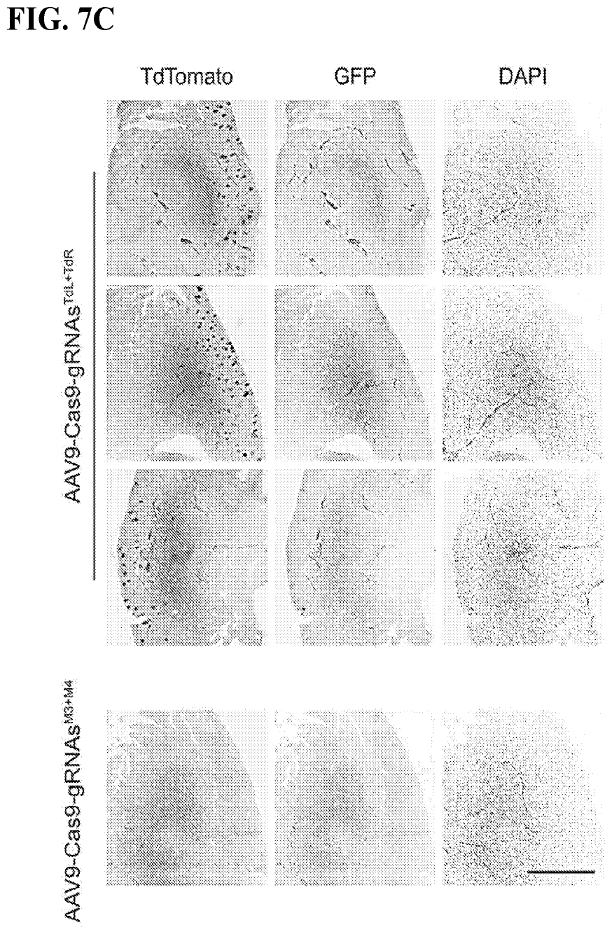

Next, to demarcate its spatial biodistribution, AAV9-Cas9-gRNAs activity was tracked at single-cell resolution, using the Ai9 mouse line (Madisen, L. et al. A robust and high-throughput Cre reporting and characterization system for the whole mouse brain. Nat Neurosci 13, 133-140, doi:10.1038/nn.2467 (2010)) that accurately couples genomic excision of a 3×Stop cassette with tdTomato fluorescence activation. Systemic delivery of AAV9-Cas9-gRNAs^{TdL+TdR} (5E11 or 4E12 vg) targeting the 3×Stop cassette generated excision-dependent tdTomato+ cells in all examined organs (FIG. 4C and FIG. 6). Targeted cells were widespread in the liver, heart and skeletal muscle (FIG. 7), arguing against clonal descent and suggesting multiple independent targeting events. Gene-edited cell clusters were detected infrequently in the brain and gonad (<0.001% of cells) (FIG. 4C), at a rate that evades detection with deep-sequencing of bulk samples (sensitivity limits ˜0.2%). Hence, AAV9-Cas9-gRNAs is robust in mice, and its widespread biodistribution also highlights that evaluation of CRISPR-Cas9 tissue-level off-targeting should complement that at the genomic-level, which has so far been the primary focus.

Furthermore, the platform enables transcriptional regulation in vivo. Mice were intramuscularly injected with the same dosage of AAV9-Cas9-VPR-gRNAs, varying only the spacer sequences to target different sets of genes. The targeted PD-L1 and CD47 genes were activated by 2-fold and 1.6-fold respectively, as determined by qRT-PCR and total mRNA-sequencing (FIGS. 4D and 4E). This demonstrates, for the first time, postnatal transcriptional regulation with CRISPR-Cas9.

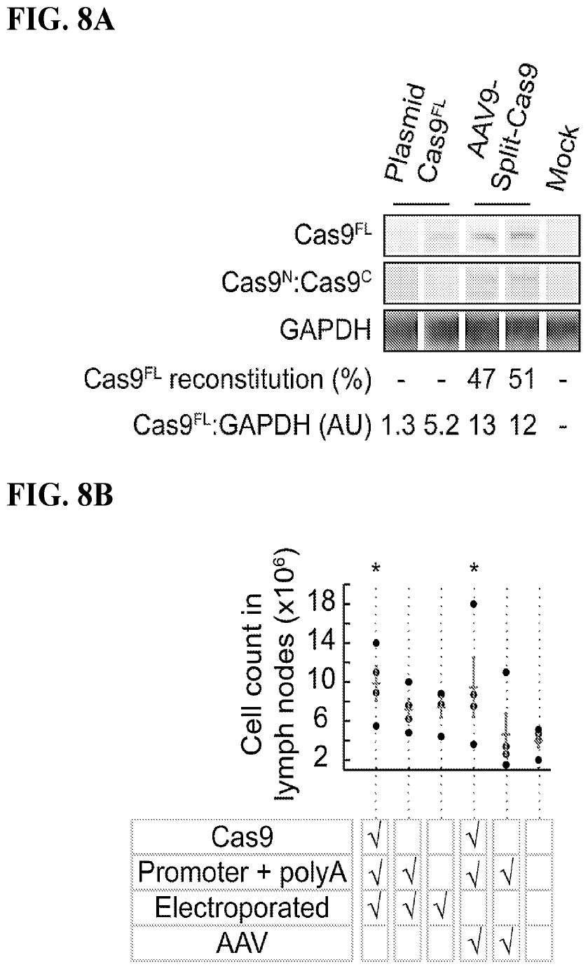

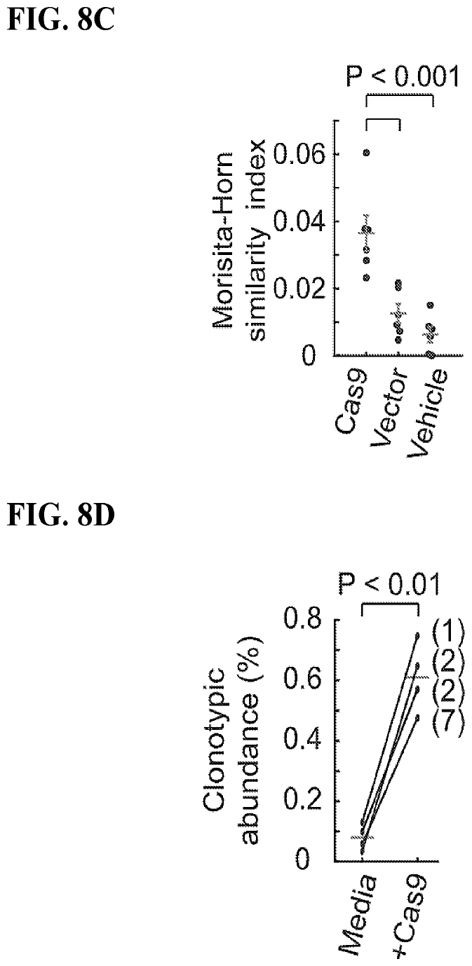

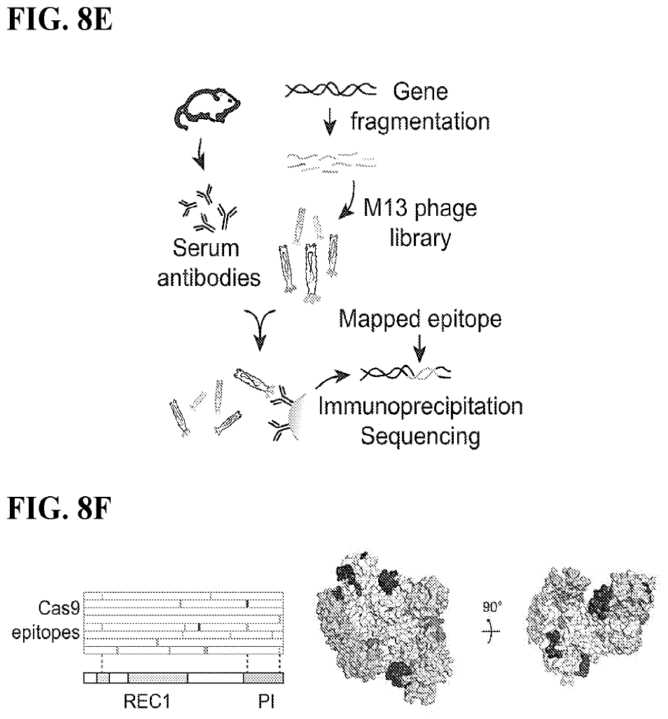

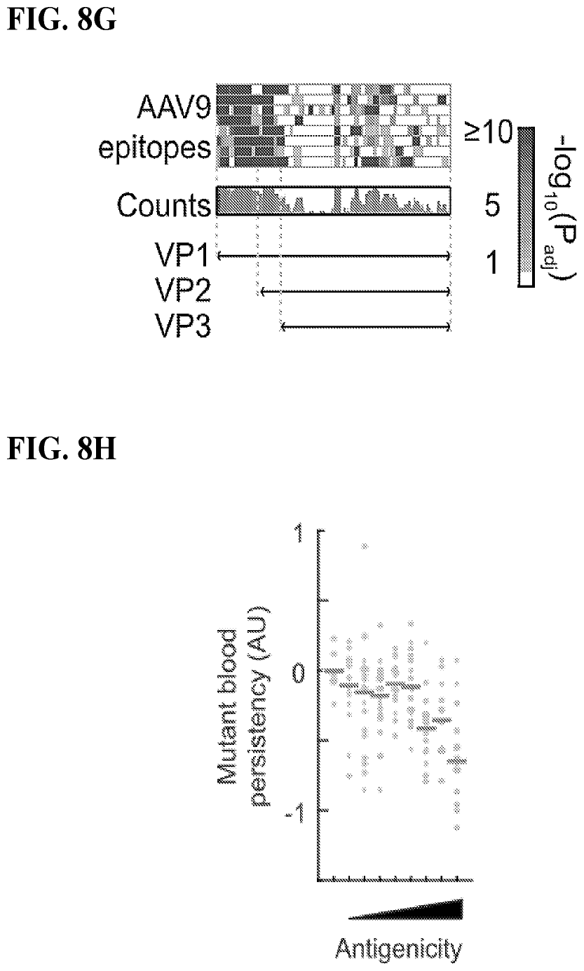

Having validated the AAV-split-Cas9 platform, it was next to examine its immunogenicity, comparing it in parallel with intramuscular DNA electroporation. Following expression of Cas9 in the adult tibialis anterior muscle (FIG. 8A), the draining lymph nodes enlarged with increased cell counts (FIG. 8B). Cellular infiltration was largely absent in the lymph nodes of mice administered the same vectors without the Cas9 coding sequence, indicating a Cas9-driven immune response. T-cells orchestrate antigen-specific immune responses, with each clonal lineage expressing a unique T-cell receptor β-chain (TCR-β) CDR3 motif that mediates most of the antigen-contact. To identify T-cell(s) mediating the cellular response, the TCR-β repertoires of lymphocytes infiltrating the draining lymph nodes were sequenced. It was observed that Cas9-exposure stimulated skewed expansion of T-cell clonotypic subsets (FIG. 8C), which implies antigen-specific T-cell activation and proliferation. Four TCR-β clonotypes were common to all Cas9-experienced animals (n=4) and undetected in all unexposed animals (n=8). Because bona fide T-cells would proliferate with antigen recall, it was sought to confirm antigen-specific T-cell expansion by challenging extracted lymphocytes with purified Cas9 protein. Of the four initial clonotypes, one was narrowed down on (Vβ16, CDR3: CASSLDRGQDTQYF) as a true Cas9-responsive T-cell clonotype, which proliferated according to Cas9 protein restimulation (FIG. 8D). Hence, Cas9 elicits cellular immunogenicity, with an antigen-specific T-cell clonotype common among injected mice (i.e., public response), and with the remaining T-cell infiltrates largely dissimilar between individuals (i.e., private response).

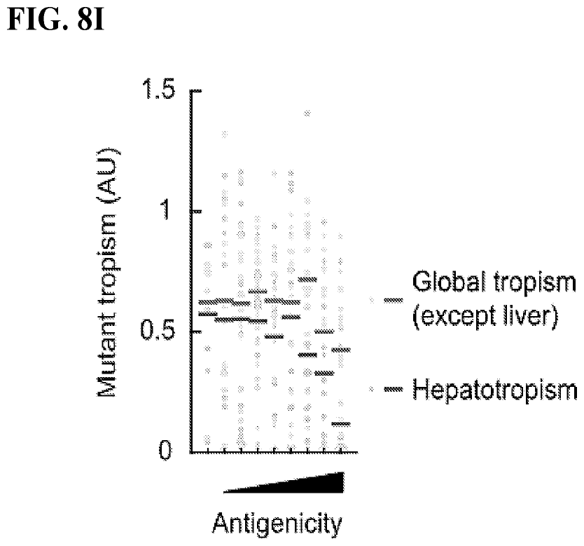

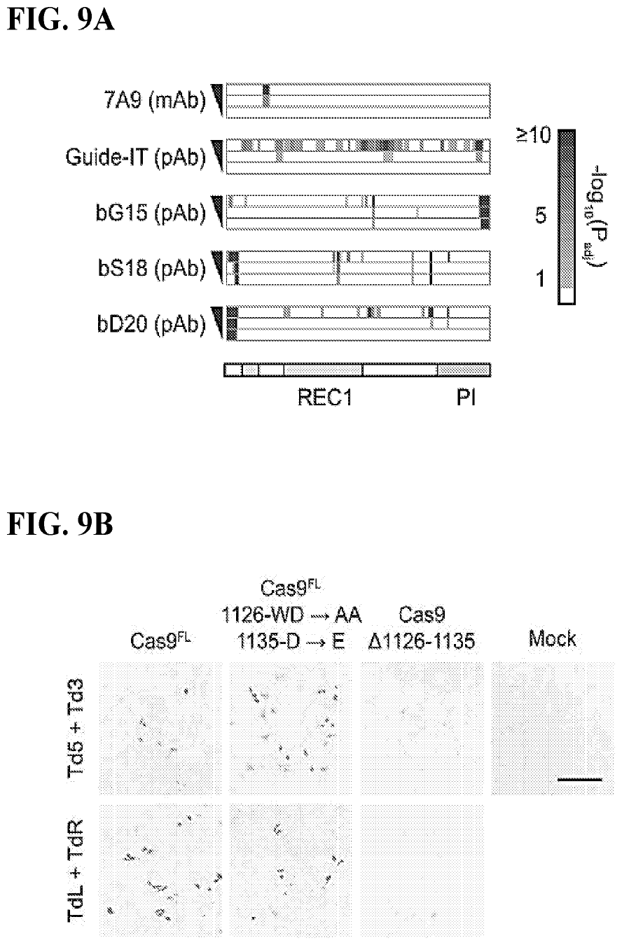

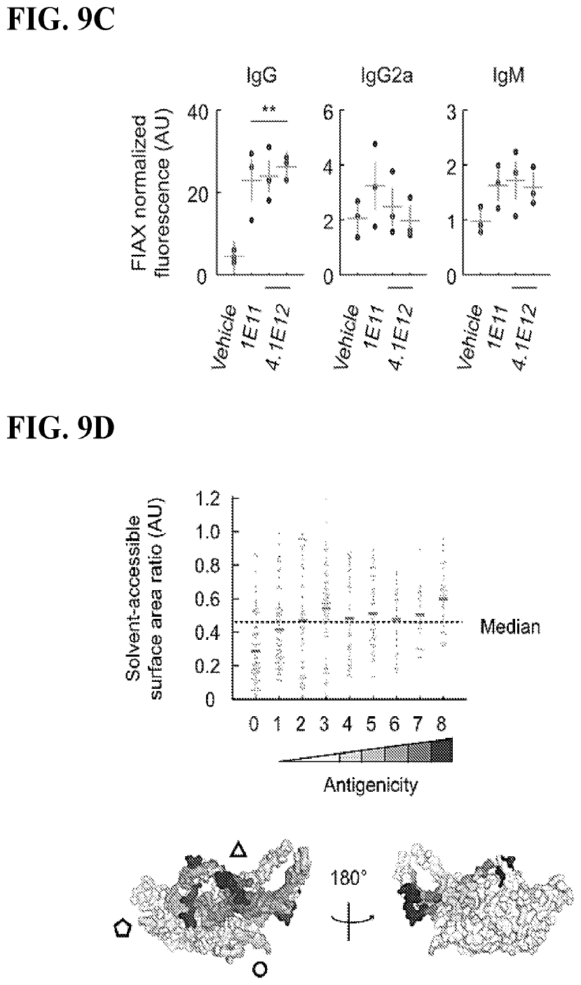

To map Cas9 antibody epitopes, serum from each animal was co-incubated with M13 phage display libraries tiling the Cas9 transgene, and antibody targets were determined by Ig:phage pull-down (FIG. 8E, FIG. 9A). Epitope mapping shows that individual Cas9-experienced animals exhibit an antibody repertoire targeting unique residues of Cas9; but three linear epitopes were observed more than once (FIG. 8F). 1352-ITGLYETRI-1360 consists of residues recognizing gRNA stem loop 2 (Nishimasu, H. et al. Crystal structure of Cas9 in complex with guide RNA and target DNA. Cell 156, 935-949, doi:10.1016/j.cell.2014.02.001 (2014)); 122-IVDEVAYHEKYP-133 resides in the REC1 domain also contributing to Cas9:gRNA interactions, but do not cover residues mediating the contact (Nishimasu, H. et al. Crystal structure of Cas9 in complex with guide RNA and target DNA. Cell 156, 935-949, doi: 10.1016/j.cell.2014.02.001 (2014)); 1126-WDPKKYGGFD-1135 resides in the PAM-binding loop and contains conserved residues, but maintains wild-type Cas9 endonucleolytic function when selectively mutated (1125-DWD→AAA (Jinek, M. et al. Structures of Cas9 endonucleases reveal RNA-mediated conformational activation. Science 343, 1247997, doi:10.1126/science.1247997 (2014)), or D1135E (Kleinstiver, B. P. et al. Engineered CRISPR-Cas9 nucleases with altered PAM specificities. Nature 523, 481-485, doi:10.1038/nature14592 (2015)) for increasing Cas9 specificity). Combining these residue changes retain Cas9 activity (FIG. 9B). This result demonstrates that identified antigenic protein regions can be altered away from the original immunogenic amino acid sequences. Future large-scale functional variant profiling could reveal more residues amenable to epitope-recoding, so as to derive less immunogenic Cas9 and Cpf1 proteins.

Unlike that with Cas9, AAV9 elicited capsid-specific antibodies (FIG. 9C) against epitopes that were shared among injected animals at surprisingly high degrees (FIG. 8G), reminiscent of a public response to viruses recently observed also in humans (Xu, G. J. et al. Viral immunology. Comprehensive serological profiling of human populations using a synthetic human virome. Science 348, aaa0698, doi: 10.1126/science.aaa0698 (2015)). Epitope-mapping provides intriguing support that AAV9 antigenicity derives from biophysical and functional aspects, instead of purely sequence-level motifs. The metastable VP1 unique and VP1/2 common regions are antigenic, suggesting their externalization from the viral interior for antigen capture. Immunodominant epitopes in VP3 lie predominantly on the capsid surface (FIG. 9D). Notably, while many of these residues can be separately double-alanine mutated without disrupting viral assembly (Adachi, K., Enoki, T., Kawano, Y., Veraz, M. & Nakai, H. Drawing a high-resolution functional map of adeno-associated virus capsid by massively parallel sequencing. Nat Commun 5, 3075, doi:10.1038/ncomms4075 (2014)), they are predominantly implicated (Adachi, K., Enoki, T., Kawano, Y., Veraz, M. & Nakai, H. Drawing a high-resolution functional map of adeno-associated virus capsid by massively parallel sequencing. Nat Commun 5, 3075, doi:10.1038/ncomms4075 (2014)) in maintaining viral blood persistency (FIG. 8H) and liver-selective tropism (FIG. 8I). Together, the immunodominant VP3 epitopes (372-FMIPQYGYLTLNDGSQAVG-390, 436-MNPLIDQYLY-445, and 494-TQNNNSEFAWPG-505) cover 12/18 of these residues associated with AAV9 hepatotropism. Hence, AAV9 elicits humoral immunogenicity that overlaps among all animals, across substantial regions of the capsid protein that modulate viral biodistribution.

To obtain a better understanding of the immunological reaction, mRNA-sequencing was next conducted on tissues from AAV9-Cas9-VPR-gRNAs and AAV9-turboRFP treated mice (4E12 and 1E11 vg), and the whole transcriptomes were compared against those from mice treated with AAV9-turboRFP only (1E11 vg). Consistent with a change in tissue composition following immune cell infiltration, differentially expressed genes are significantly enriched for immunological gene ontology processes. CD8+ T-cells are particular intriguing, because they effect tissue damage by cytolysis. However, a closer look at genes encoding key differentiation signals (e.g. IL-12, Ifn-α/β, IL-2) and cytolytic effector proteins (e.g. Prfl, Gmzb, FasL) revealed that these were not altered at statistically significant levels. This led to examining functional readouts more sensitively by intra-tissue immunofluorescence and histology.

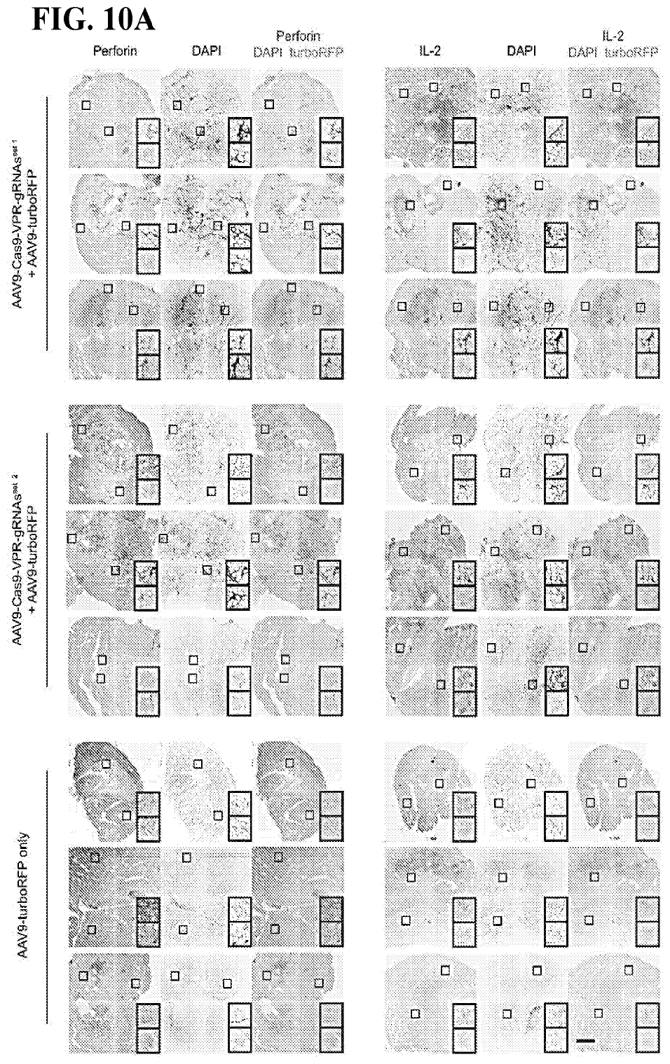

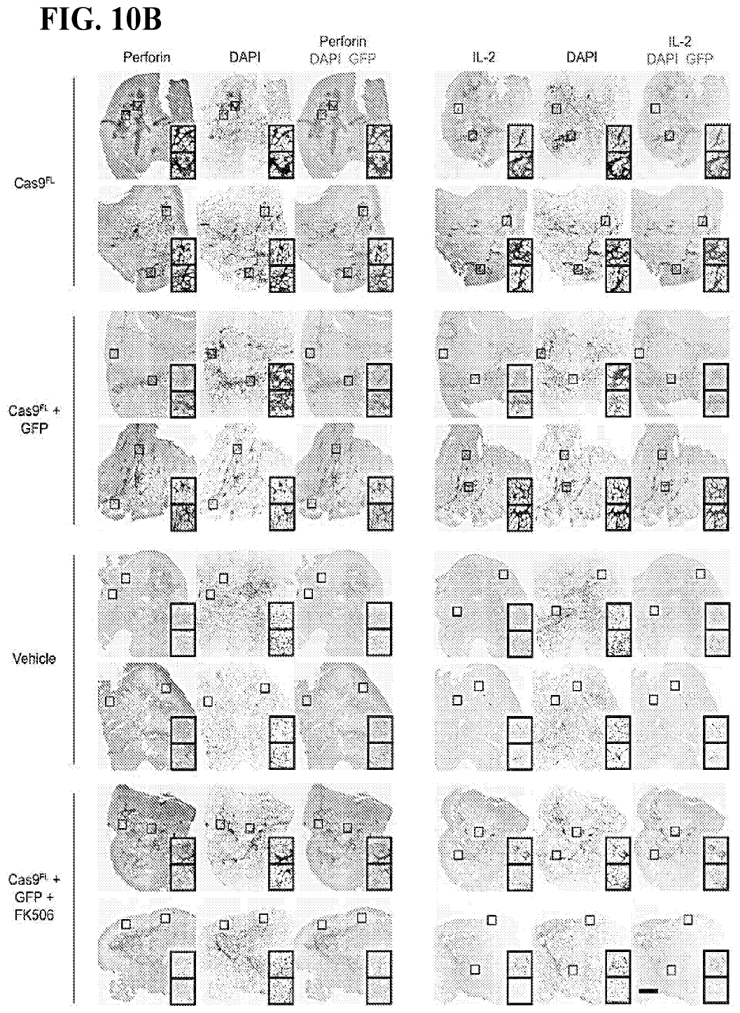

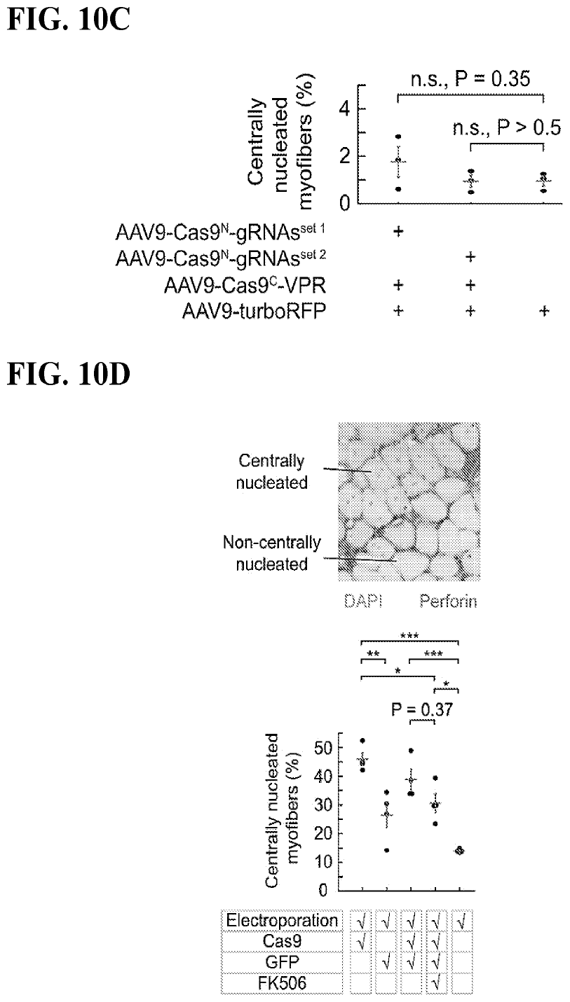

Interleukin-2 (IL-2) is pivotal for cytolytic T-cell differentiation (Pipkin, M. E. et al. Interleukin-2 and inflammation induce distinct transcriptional programs that promote the differentiation of effector cytolytic T cells. Immunity 32, 79-90, doi:10.1016/j.immuni.2009.11.012 (2010)). Downstream, perforin (Prfl) is the essential pore-forming protein released by cytolytic T lymphocytes and NK cells to destroy target cells. In line with mRNA-sequencing, immunofluorescence indicated minimal IL-2 or perforin protein secretion within AAV9-Cas9-VPR-gRNAs-injected muscles (FIG. 10A). Absence of elevated perforin release suggests minimal downstream cytolysis. Consistent with this, there was an absence of overt histological damage (FIG. 10C), in stark contrast to that seen with DNA electroporation. IL-2 and perforin were both strongly elevated in muscles electroporated with Cas9-encoding DNA and myofiber degeneration-repair clearly visible (FIGS. 10B and 10D). This cellular damage is not due to simple physical disruptions from DNA electroporation, because IL2 and perforin levels and myofiber cellular damage were significantly increased over that in vehicle-electroporated muscles and could be alleviated by the administration of immunosuppressant (FK506), indicating an immunological basis. Hence, although AAV-CRISPR-Cas9 activates the host immune system, it does not trigger overt cellular damage observed with alternative delivery methods.

CRISPR-Cas9 allows user-defined DNA-RNA-protein interactions, driving a wide range of applications that includes epigenetic regulation and protein-complex recruitment. The use of CRISPR-Cas9 in vivo has the potential not just to correct genetic defects, but also to modulate the epigenome. Split-Cas9 shortens the coding sequence below that of all known Cas9 orthologs, allowing facile domain-fusions that AAV-Cas9^{FL}s are unable to accommodate in their current forms (Ran, F. A. et al. In vivo genome editing using Staphylococcus aureus Cas9. Nature 520, 186-191, doi:10.1038/nature14299 (2015); Nelson, C. E. et al. In vivo genome editing improves muscle function in a mouse model of Duchenne muscular dystrophy. Science 351, 403-407, doi:10.1126/science.aad5143 (2016); Tabebordbar, M. et al. In vivo gene editing in dystrophic mouse muscle and muscle stem cells. Science 351, 407-411, doi:10.1126/science.aad5177 (2016); Long, C. et al. Postnatal genome editing partially restores dystrophin expression in a mouse model of muscular dystrophy. Science 351, 400-403, doi:10.1126/science.aad5725 (2016); Yang, Y. et al. A dual AAV system enables the Cas9-mediated correction of a metabolic liver disease in newborn mice. Nature biotechnology, doi:10.1038/nbt.3469 (2016); Yin, H. et al. Therapeutic genome editing by combined viral and non-viral delivery of CRISPR system components in vivo. Nature biotechnology, doi:10.1038/nbt.3471 (2016); Senis, E. et al. CRISPR/Cas9-mediated genome engineering: an adeno-associated viral (AAV) vector toolbox. Biotechnology journal 9, 1402-1412, doi:10.1002/biot.201400046 (2014); Swiech, L. et al. In vivo interrogation of gene function in the mammalian brain using CRISPR-Cas9. Nature biotechnology 33, 102-106, doi:10.1038/nbt.3055 (2015)). This platform enables in vivo applications including and beyond that of genome-editing, which has so far only begin to demonstrate the power of CRISPR-Cas9 in manipulating live mammals.

The fundamental consideration for clinical implementation of AAV-CRISPR-Cas9 lies in its safety. Alternative delivery methods such as DNA electroporation and adenoviruses (Wang, D. et al. Adenovirus-Mediated Somatic Genome Editing of Pten by CRISPR/Cas9 in Mouse Liver in Spite of Cas9-Specific Immune Responses. Hum Gene Ther 26, 432-442, doi:10.1089/hum.2015.087 (2015)) cause severe inflammation and immunological reactions within the host. Inherently benign delivery vectors are hence particularly attractive. Precedents of immune evasion/tolerance by viruses have been documented in experimental and endogenous settings, mediated by processes ranging through insufficient activation, sub-threshold inflammatory milieu, and/or active immunosuppression (Mays, L. E. & Wilson, J. M. The complex and evolving story of T cell activation to AAV vector-encoded transgene products. Mol Ther 19, 16-27, doi:10.1038/mt.2010.250 (2011); Zajac, A. J. et al. Viral immune evasion due to persistence of activated T cells without effector function. J Exp Med 188, 2205-2213 (1998); Curtsinger, J. M., Lins, D. C. & Mescher, M. F. Signal 3 determines tolerance versus full activation of naive CD8 T cells: dissociating proliferation and development of effector function. J Exp Med 197, 1141-1151, doi:10.1084/jem.20021910 (2003)). This example shows that AAV-CRISPR-Cas9 activates immune responses without overt cellular damage. This attests to its comparatively favorable safety profile, both for use in research models and ultimately human patients.

Constructs and sequences. U6-driven gRNA plasmids were constructed as described (Mali, P. et al. RNA-guided human genome engineering via Cas9. Science 339, 823-826, doi:10.1126/science.1232033 (2013)). AAV plasmid backbone was derived from pZac2.1-CASI-EGFP-RGB, a gift from Luk Vandenberghe. Minicircles parental plasmids were cloned in ZYCY10P3S2T, and minicircles were generated as described (Kay, M. A., He, C. Y. & Chen, Z. Y. A robust system for production of minicircle DNA vectors. Nature biotechnology 28, 1287-1289, doi:10.1038/nbt.1708 (2010)). AAV plasmids were cloned in Stbl3 (Life Technologies C7373-03). All other plasmids were cloned in DH5a (NEB C2987H). Protein transgenes were expressed from ubiquitous hybrid promoters: SMVP promoter (generated by fusing SV40 enhancer-CMV-promoter-chimeric intron), CASI promoter (Balazs, A. B. et al. Antibody-based protection against HIV infection by vectored immunoprophylaxis. Nature 481, 81-84, doi:10.1038/nature10660 (2012)), or CAG promoter (Matsuda, T. & Cepko, C. L. Electroporation and RNA interference in the rodent retina in vivo and in vitro. Proceedings of the National Academy of Sciences of the United States of America 101, 16-22, doi:10.1073/pnas.2235688100 (2004)). SMVP plasmid was derived from pMAXGFP (Lonza). pCAG-GFP was a gift from Connie Cepko (Addgene plasmid #11150). pAAV-CMV-HI-EGFP-Cre-WPRE-SV40pA was obtained from the University of Pennsylvania Vector Core.

AAV Packaging and Purification.

AAVs were packaged via the triple-transfection method (Grieger, J. C., Choi, V. W. & Samulski, R. J. Production and characterization of adeno-associated viral vectors. Nat Protoc 1, 1412-1428, doi:10.1038/nprot.2006.207 (2006); Zolotukhin, S. et al. Recombinant adeno-associated virus purification using novel methods improves infectious titer and yield. Gene Ther 6, 973-985, doi:10.1038/sj.gt.3300938 (1999)). HEK293 cells (Cell Biolabs AAV-100 or Agilent 240073) were plated in growth media consisting of DMEM+glutaMAX+pyruvate+10% FBS (Life Technologies), supplemented with 1×MEM non-essential amino acids (Gibco). Confluency at transfection was between 70-90%. Media was replaced with fresh pre-warmed growth media before transfection. For each 15-cm dish, 20 μg of pHelper (Cell Biolabs), 10 μg of pRepCap [encoding capsid proteins for AAV-DJ (Cell Biolabs) or AAV9 (UPenn Vector Core)], and 10 μg of pAAV were mixed in 500 μl of DMEM, and 200 μg of PEI “MAX” (Polysciences) (40 kDa, 1 mg/ml in H₂O, pH 7.1) added for PEI:DNA mass ratio of 5:1. The mixture was incubated for 15 min., and transferred drop-wise to the cell media. For large-scale AAV production, HYPERFlask ‘M’ (Corning) were used, and the transfection mixture consisted of 200 μg of pHelper, 100 μg of pRepCap, 100 μg of pAAV, and 2 mg of PEIMAX. The day after transfection, media was changed to DMEM+glutamax+pyruvate+2% FBS. Cells were harvested 48-72 hrs after transfection by scrapping or dissociation with 1×PBS (pH7.2)+5 mM EDTA, and pelleted at 1500 g for 12 min. Cell pellets were resuspended in 1-5 ml of lysis buffer (Tris HCl pH 7.5+2 mM MgCl+150 mM NaCl), and freeze-thawed 3× between dry-ice-ethanol bath and 37° C. water bath. Cell debris was clarified via 4000 g for 5 min., and the supernatant collected. Downstream processing differed depending on applications.

For preparation of AAV-containing lysates, the collected supernatant was treated with 50 U/ml of Benzonase (Sigma-Aldrich) and 1 U/ml of Riboshredder (Epicentre) for 30 min. at 37° C. to remove unpackaged nucleic acids, filtered through a 0.45 μm PVDF filter (Millipore), and used directly on cells or stored in −80° C.

For purification of AAV via chloroform-ammonium sulfate precipitation, 1/10^{th} volume of chloroform and NaCl (1 M final concentration) was added to the lysate and shaken vigorously. After centrifugation, the supernatant was incubated with PEG-8000 (10% final w/v) on ice for ≥1 hr or overnight. PEG-precipitated virions were centrifuged (4000 g, 30 min., 4° C.), and resuspended in 50 mM HEPES buffer (pH8). 50 U/ml of Benzonase (Sigma-Aldrich) and 1 U/ml of Riboshredder (Epicentre) were added and incubated for 30 min. at 37° C. An equal volume of chloroform was then added, and vigorously vortexed. After centrifugation, the aqueous phase was collected and residual chloroform evaporated for 30 min. Ammonium sulfate precipitation of AAVs were performed with a 0.5 M-2 M cut-off. AAVs were then resuspended and dialyzed in 1×PBS+35 mM NaCl, quantified for viral titers, and stored in −80° C.

All experiments with purified AAVs utilized the iodixanol density gradient ultracentrifugation purification method (Grieger, J. C., Choi, V. W. & Samulski, R. J. Production and characterization of adeno-associated viral vectors. Nat Protoc 1, 1412-1428, doi:10.1038/nprot.2006.207 (2006); Zolotukhin, S. et al. Recombinant adeno-associated virus purification using novel methods improves infectious titer and yield. Gene Ther 6, 973-985, doi:10.1038/sj.gt.3300938 (1999)), unless otherwise stated. The collected AAV supernatant was first treated with 50 U/ml Benzonase and 1 U/ml Riboshredder for 30 min. at 37° C. After incubation, the lysate was concentrated to <3 ml by ultrafiltration with Amicon Ultra-15 (50 kDa MWCO) (Millipore), and loaded on top of a discontinuous density gradient consisting of 2 ml each of 15%, 25%, 40%, 60% Optiprep (Sigma-Aldrich) in an 11.2 ml Optiseal polypropylene tube (Beckman-Coulter). The tubes were ultracentrifuged at 58000 rpm, at 18° C., for 1.5 hr, on an NVT65 rotor. The 40% fraction was extracted, and dialyzed with 1×PBS (pH 7.2) supplemented with 35 mM NaCl, using Amicon Ultra-15 (50 kDa or 100 kDa MWCO) (Millipore). The purified AAVs were quantified for viral titers, and stored in −80° C.

AAV2/9-CMV-HI-EGFP-Cre-WPRE-SV40 (Lot V4565MI-R), AAV2/9-CB7-CI-EGFP-WPRE-rBG [Lot CS0516(293)], AAV2/9-CB7-CI-mCherry-WPRE-rBG (Lot V4571MI-R), and AAV2/9-CMV-turboRFP-WPRE-rBG (Lot V4528MI-R-DL) were obtained from the University of Pennsylvania Vector Core.

AAV titers (vector genomes) were quantified via hydrolysis-probe qPCR (Aurnhammer, C. et al. Universal real-time PCR for the detection and quantification of adeno-associated virus serotype 2-derived inverted terminal repeat sequences. Human gene therapy methods 23, 18-28, doi:10.1089/hgtb.2011.034 (2012)) against standard curves generated from linearized parental AAV plasmids.

TABLE 1
A list of gRNA spacer sequences used in this study.

| | Spacer | gRNAs set (for |
|---|---|---|
| Sp gRNAs | sequence, including 5′ G from U6 promoter | AAV9-Cas9-VPR) |
| Acvr2b B1 | GGGCCATGTGGACATCCATGAGGTGAGACAGTGCCAGCGT | |
| Acvr2b B3 | GGCCTGAAGCCACTACAGCTGCTGGAGATCAAGGCTCG | |
| Acvr2a A3 | GGCCCTAGCATCTAAGTTCTCGCAGGC | |
| Acvr2a A4 | GGTCATTCCATCTCAGCTGTGACAGCAGCGCAGAA | |
| Mstn M3 | GTCAAGCCCAAAGTCTCTCCGGGACCTCTT | 1 and 2 |
| Mstn M4 | GGAATCCCGGTGCTGCCGCTACCCCCTCA | 1 and 2 |
| Ai9 Td5 | GCTAGAGAATAGGAACTTCTT | |
| Ai9 TdL | GAAAGAATTGATTTGATACCG | |
| Ai9 Td3 | GATCCCCATCAAGCTGATC | |
| Ai9 TdR | GGTATGCTATACGAAGTTATT | |
| PD-L1 P1 | GCTCGAGATAAGACC | 1 |
| PD-L1 P2 | GCTAAAGTCATCCGC | 1 |
| FST F1 | GGTTCTTATTTGCGT | |
| FST F2 | GGAAATCAAAGCGGC | 1 and 2 |
| CD47 C1 | GAAGGAGTTCCTCGG | 1 |
| CD47 C2 | GAGGAGGTCCACTTC | 1 |

TABLE 2
A list of locus-specific genotyping primers for deep-sequencing used in this study.

| Target locus | Sequence |
|---|---|
| Acvr2b F | CTTTCCCTACACGACGCTCTTCCGATCTNNNNNNCTGGAGTGTTAGAGTGGGCG |
| Acvr2b R | GGAGTTCAGACGTGTGCTCTTCCGATCTGACTGCCCCATGGAAAGACA |
| Mstn F | CTTTCCCTACACGACGCTCTTCCGATCTNNNNNNGGGCCATGAAAGGAAAAATGAAGT |
| Mstn R | GGAGTTCAGACGTGTGCTCTTCCGATCTGCCTCTGGGGTTTGCTTGGT |
| Acvr2a F | CTTTCCCTACACGACGCTCTTCCGATCTNNNNNNGAGATATAAGCTGAATAAGGCCAATGACATACT |
| Acvr2a R | GGAGTTCAGACGTGTGCTCTTCCGATCTCTACTGCTCTTTCCTGCCGA |

TABLE 3
A list of qPCR probes and primers used in this
study.

| Target locus | Sequence |
|---|---|
| Acyr2b F | GCCTACTCGCTGCTGCCCATT |
| Acvr2b R | CCTGGAGACCCCCAAAAGCTC |
| Acvr2b probe | /5HEX/AGATCT+TC+CC+AC+TT+CA+GGT/ |
| | 3IABkFQ/ |
| AAV ITR F | GGAACCCCTAGTGATGGAGTT |
| AAV ITR R | CGGCCTCAGTGAGCGA |
| AAV ITR probe | /56-FAM/CACTCCCTCTCTGCGCGCTCG/3BH |

Coding sequences of split-Cas9.

Coding sequence for SpCas9^{N}-RmaIntN:
MAPKKKRKVGIHGVPAADKKYSIGLDIGTNSVGWAVITDEYKVPSKKFKVLGNTDRHSIKKNLIGALLFDSGETAEATRLKRTARRRYTRRKNR
ICYLQEIFSNEMAKVDDSFFHRLEESFLVEEDKKHERHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAILRRQEDFYPFLKDNREKIEKILTFRIPYYVGP
LARGNSRFAWMTRKSEETITPWNFEEVVDKGASAQSFIERMTNFDKNLPNEKVLPKHSLLYEYFTVYNELTKVKYVTEGMRKPAFLSGEQKKAI
VDLLFKTNRKVTVKQLKEDYFKKIECFDSVEISGVEDRFNASLGTYHDLLKIIKDKDFLDNEENEDILEDIVLTLTLFEDREMIEERLKTYAHL
FDDKVMKQLKRRRYTGWGRLSRKLINGIRDKQSGKTILDFLKSDGFANRNFMQLIHDDSLTFKEDIQKAQVC [symbol] [symbol] [symbol] [symbol] [symbol] [symbol] [symbol]
.
ATGGCCCCAAAGAAGAAGCGGAAGGTCGGTATCCACGGAGTCCCAGCAGCCGACAAGAAGTACTCCATTGGGCTCGATAT
CGGCACAAACAGCGTCGGCTGGGCCGTCATTACGGACGAGTACAAGGTGCCGAGCAAAAAATTCAAAGTTCTGGGCAATA
CCGATCGCCACAGCATAAAGAAGAACCTCATTGGCGCCCTCCTGTTCGACTCCGGGGAAACGGCCGAAGCCACGCGGCT
CAAAAGAACAGCACGGCGCAGATATACCCGCAGAAAGAATCGGATCTGCTACCTGCAGGAGATCTTTAGTAATGAGATGG
CTAAGGTGGATGACTCTTTCTTCCATAGGCTGGAGGAGTCCTTTTTGGTGGAGGAGGATAAAAAGCACGAGCGCCACCCA
ATCTTTGGCAATATCGTGGACGAGGTGGCGTACCATGAAAAGTACCCAACCATATATCATCTGAGGAAGAAGCTTGTAGAC
AGTACTGATAAGGCTGACTTGCGGTTGATCTATCTCGCGCTGGCGCATATGATCAAATTTCGGGGACACTTCCTCATCGAG
GGGGACCTGAACCCAGACAACAGCGATGTCGACAAACTCTTTATCCAACTGGTTCAGACTTACAATCAGCTTTTCGAAGAG
AACCCGATCAACGCATCCGGAGTTGACGCCAAAGCAATCCTGAGCGCTAGGCTGTCCAAATCCCGGCGGCTCGAAAACCT
CATCGCACAGCTCCCTGGGGAGAAGAAGAACGGCCTGTTTGGTAATCTTATCGCCCTGTCACTCGGGCTGACCCCCAACT
TTAAATCTAACTTCGACCTGGCCGAAGATGCCAAGCTTCAACTGAGCAAAGACACCTACGATGATGATCTCGACAATCTGC
TGGCCCAGATCGGCGACCAGTACGCAGACCTTTTTTTGGCGGCAAAGAACCTGTCAGACGCCATTCTGCTGAGTGATATT
CTGCGAGTGAACACGGAGATCACCAAAGCTCCGCTGAGCGCTAGTATGATCAAGCGCTATGATGAGCACCACCAAGACTT
GACTTTGCTGAAGGCCCTTGTCAGACAGCAACTGCCTGAGAAGTACAAGGAAATTTTCTTCGATCAGTCTAAAAATGGCTA
CGCCGGATACATTGACGGCGGAGCAAGCCAGGAGGAATTTTACAAATTTATTAAGCCCATCTTGGAAAAAATGGACGGCAC
CGAGGAGCTGCTGGTAAAGCTTAACAGAGAAGATCTGTTGCGCAAACAGCGCACTTTCGACAATGGAAGCATCCCCCACC
AGATTCACCTGGGCGAACTGCACGCTATCCTCAGGCGGCAAGAGGATTTCTACCCCTTTTTGAAAGATAACAGGGAAAAGA
TTGAGAAAATCCTCACATTTCGGATACCCTACTATGTAGGCCCCCTCGCCCGGGGAAATTCCAGATTCGCGTGGATGACTC
GCAAATCAGAAGAGACCATCACTCCCTGGAACTTCGAGGAAGTCGTGGATAAGGGGGCCTCTGCCCAGTCCTTCATCGAA
AGGATGACTAACTTTGATAAAAATCTGCCTAACGAAAAGGTGCTTCCTAAACACTCTCTGCTGTACGAGTACTTCACAGTTT
ATAACGAGCTCACCAAGGTCAAATACGTCACAGAAGGGATGAGAAAGCCAGCATTCCTGTCTGGAGAGCAGAAGAAAGCT
ATCGTGGACCTCCTCTTCAAGACGAACCGGAAAGTTACCGTGAAACAGCTCAAAGAAGACTATTTCAAAAAGATTGAATGTT
TCGACTCTGTTGAAATCAGCGGAGTGGAGGATCGCTTCAACGCATCCCTGGGAACGTATCACGATCTCCTGAAAATCATTA
AAGACAAGGACTTCCTGGACAATGAGGAGAACGAGGACATTCTTGAGGACATTGTCCTCACCCTTACGTTGTTTGAAGATA
GGGAGATGATTGAAGAACGCTTGAAAACTTACGCTCATCTCTTCGACGACAAAGTCATGAAACAGCTCAAGAGGCGCCGAT
ATACAGGATGGGGGCGGCTGTCAAGAAAACTGATCAATGGGATCCGAGACAAGCAGAGTGGAAAGACAATCCTGGATTTT
CTTAAGTCCGATGGATTTGCCAACCGGAACTTCATGCAGTTGATCCATGATGACTCTCTCACCTTTAAGGAGGACATCCAG
AAAGCACAAGTT

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

TGA
Coding sequence for RmaIntC-SpCas9^{C}-P2A-turboGFP: [symbol] [symbol] [symbol] [symbol]
SGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGDSRADPKKKRKVSRAGSGATNFSLLKQAGDVEENPGPMPAMKIECR
ITGTLNGVEFELVGGGEGTPEQGRMTNKNKSTKGALTFSPYLLSHVMGYGFYHFGTYPSGYENPFLHAINNGGYTNTRIEKYEDGGVLHVSFSY
RYEAGRVIGDFKVVGTGFPEDSVIFTDKIIRSNATVEHLHPMGDNVLVGSFARTFSLRDGGYYSFVVDSHMHFKSAIHPSILQNGGPMFAFRRV
EELHSNTELGIVEYQHAFKTPIAFARSRAR.

[symbol]

[symbol]

[symbol]

[symbol]

[symbol]

[symbol]

[symbol]

[symbol]

[symbol]

[symbol]

T
CTGGCCAGGGGGACAGTCTTCACGAGCACATCGCTAATCTTGCAGGTAGCCCAGCTATCAAAAAGGGAATACTGCAGACC
GTTAAGGTCGTGGATGAACTCGTCAAAGTAATGGGAAGGCATAAGCCCGAGAATATCGTTATCGAGATGGCCCGAGAGAA
CCAAACTACCCAGAAGGGACAGAAGAACAGTAGGGAAAGGATGAAGAGGATTGAAGAGGGTATAAAAGAACTGGGGTCCC
AAATCCTTAAGGAACACCCAGTTGAAAACACCCAGCTTCAGAATGAGAAGCTCTACCTGTACTACCTGCAGAACGGCAGGG
ACATGTACGTGGATCAGGAACTGGACATCAATCGGCTCTCCGACTACGACGTGGATCATATCGTGCCCCAGTCTTTTCTCA
AAGATGATTCTATTGATAATAAAGTGTTGACAAGATCCGATAAAAATAGAGGGAAGAGTGATAACGTCCCCTCAGAAGAAGT
TGTCAAGAAAATGAAAAATTATTGGCGGCAGCTGCTGAACGCCAAACTGATCACACAACGGAAGTTCGATAATCTGACTAA
GGCTGAACGAGGTGGCCTGTCTGAGTTGGATAAAGCCGGCTTCATCAAAAGGCAGCTTGTTGAGACACGCCAGATCACCA
AGCACGTGGCCCAAATTCTCGATTCACGCATGAACACCAAGTACGATGAAAATGACAAACTGATTCGAGAGGTGAAAGTTA
TTACTCTGAAGTCTAAGCTGGTCTCAGATTTCAGAAAGGACTTTCAGTTTTATAAGGTGAGAGAGATCAACAATTACCACCA
TGCGCATGATGCCTACCTGAATGCAGTGGTAGGCACTGCACTTATCAAAAAATATCCCAAGCTTGAATCTGAATTTGTTTAC
GGAGACTATAAAGTGTACGATGTTAGGAAAATGATCGCAAAGTCTGAGCAGGAAATAGGCAAGGCCACCGCTAAGTACTTC
TTTTACAGCAATATTATGAATTTTTTCAAGACCGAGATTACACTGGCCAATGGAGAGATTCGGAAGCGACCACTTATCGAAA
CAAACGGAGAAACAGGAGAAATCGTGTGGGACAAGGGTAGGGATTTCGCGACAGTCCGGAAGGTCCTGTCCATGCCGCA
GGTGAACATCGTTAAAAAGACCGAAGTACAGACCGGAGGCTTCTCCAAGGAAAGTATCCTCCCGAAAAGGAACAGCGACA
AGCTGATCGCACGCAAAAAAGATTGGGACCCCAAGAAATACGGCGGATTCGATTCTCCTACAGTCGCTTACAGTGTACTGG
TTGTGGCCAAAGTGGAGAAAGGGAAGTCTAAAAAACTCAAAAGCGTCAAGGAACTGCTGGGCATCACAATCATGGAGCGA
TCAAGCTTCGAAAAAAACCCCATCGACTTTCTCGAGGCGAAAGGATATAAAGAGGTCAAAAAAGACCTCATCATTAAGCTTC
CCAAGTACTCTCTCTTTGAGCTTGAAAACGGCCGGAAACGAATGCTCGCTAGTGCGGGCGAGCTGCAGAAAGGTAACGAG
CTGGCACTGCCCTCTAAATACGTTAATTTCTTGTATCTGGCCAGCCACTATGAAAAGCTCAAAGGGTCTCCCGAAGATAATG
AGCAGAAGCAGCTGTTCGTGGAACAACACAAACACTACCTTGATGAGATCATCGAGCAAATAAGCGAATTCTCCAAAAGAG
TGATCCTCGCCGACGCTAACCTCGATAAGGTGCTTTCTGCTTACAATAAGCACAGGGATAAGCCCATCAGGGAGCAGGCA
GAAAACATTATCCACTTGTTTACTCTGACCAACTTGGGCGCGCCTGCAGCCTTCAAGTACTTCGACACCACCATAGACAGA
AAGCGGTACACCTCTACAAAGGAGGTCCTGGACGCCACACTGATTCATCAGTCAATTACGGGGCTCTATGAAACAAGAATC
GACCTCTCTCAGCTCGGTGGAGACAGCAGGGCTGACCCCAAGAAGAAGAGGAAGGTGTCTCGAGCTGGATCCGGAGCCA
CGAACTTCTCTCTGTTAAAGCAAGCAGGGGACGTGGAAGAAAACCCCGGTCCTATGCCCGCCATGAAGATCGAGTGCC
GCATCACCGGCACCCTGAACGGCGTGGAGTTCGAGCTGGTGGGCGGCGGAGAGGGCACCCCCGAGCAGGGCCGCAT
GACCAACAAGATGAAGAGCACCAAAGGCGCCCTGACCTTCAGCCCCTACCTGCTGAGCCACGTGATGGGCTACGGCTT
CTACCACTTCGGCACCTACCCCAGCGGCTACGAGAACCCCTTCCTGCACGCCATCAACAACGGCGGCTACACCAACAC
CCGCATCGAGAAGTACGAGGACGGCGGCGTGCTGCACGTGAGCTTCAGCTACCGCTACGAGGCCGGCCGCGTGATCG
GCGACTTCAAGGTGGTGGGCACCGGCTTCCCCGAGGACAGCGTGATCTTCACCGACAAGATCATCCGCAGCAACGCCA
CCGTGGAGCACCTGCACCCCATGGGCGATAACGTGCTGGTGGGCAGCTTCGCCCGCACCTTCAGCCTGCGCGACGGC
GGCTACTACAGCTTCGTGGTGGACAGCCACATGCACTTCAAGAGCGCCATCCACCCCAGCATCCTGCAGAACGGGGGC
CCCATGTTCGCCTTCCGCCGCGTGGAGGAGCTGCACAGCAACACCGAGCTGGGCATCGTGGAGTACCAGCACGCCTTC
AAGACCCCCATCGCCTTCGCCAGATCTCGAGCTCGATGA
Coding sequence for RmaIntC-SpCas9^{C}-3PR: [symbol] [symbol] [symbol]
SGQGDSLHEHIANLAGSPAIKKGILQTVKVVDELVKVMGRHK
PENIVIEMARENQTTQKGQKNSRERMKRIEEGIKELGSQILKEHPVENTQLQNEKLYLYYLQNGRDMYVDQELDINRLSDYDVDHIVPQSFLKD
DSIDNKVLTRSDKNRGKSDNVPSEEVVKKMKNYWRQLLNAKLITQRKFDNLTKAERGGLSELDKAGFIKRQLVETRQITKHVAQILDSRMNTKY
DENDKLIREVKVITLKSKLVSDFRKDFQFYKVREINNYHHAHDAYLNAVVGTALIKKYPKLESEFVYGDYKVYDVRKMIAKSEQEIGKATAKYF
FYSNIMNFFKTEITLANGEIRKRPLIETNGETGEIVWDKGRDFATVRKVLSMPQVNIVKKTEVQTGGFSKESILPKRNSDKLIARKKDWDPKKY
GGFDSPTVAYSVLVVAKVEKGKSKKLKSVKELLGITIMERSSFEKNPIDFLEAKGYKEVKKDLIIKLPKYSLFELENGRKRMLASAGELQKGNE
LALPSKYVNFLYLASHYEKLKGSPEDNEQKQLFVEQHKHYLDEIIEQISEFSKRVILADANLDKVLSAYNKHRDKPIREQAENIIHLFTLTNLG
APAAFKYFDTTIDRKRYTSTKEVLDATLIHQSITGLYETRIDLSQLGGDSRADPKKKRKVSPGIRRLDALISTSLYKKAGYKEASGSGRADALD
DFDLDMIGSDALDDFDLDMIGSDALDDFDLDMLGSDALDDFDLDMLINSRSSGSPKKKRKVGSQYLPDTDDRHRIEEKRKRTYETFKSIMKKSP
FSGPTDPRPPPRRIAVPSRSSASVPKPAPQPYPFTSSLSTINYDEFPTMVFPSGQISQASALAPAPPQVLPQAPAPAPAPAMVSALAQAPAPVP
VLAPGPPQAVAPPAPKPTQAGEGTLSEALLQLQFDDEDLGALLGNSTDPAVFTDLASVDNSEFQQLLNQGIPVAPHTTEPMINEYPEAITRLVT
GAQRPPDPAPAPLGAPGLPNGLLSGDEDFSSIADMDFSALLGSGSGSRDSREGMFLPKPEAGSAISDVFEGREVCQPKRIRPFHPPGSPWANRP
LPASLAPTPTGPVHEPVGSLTPAPVPQPLDPAPAVTPEASHLLEDPDEETSQAVKALREMADTVIPQKEEAAICGQMDLSHPPPRGHLDELTTT
LESMTEDLNLDSPLTPELNEILDTFLNDECLLHAMHISTGLSIFDTSLF.

[symbol]

[symbol]

[symbol]

[symbol]

[symbol]

[symbol]

[symbol]

[symbol]

[symbol]

[symbol]

T
CTGGCCAGGGGGACAGTCTTCACGAGCACATCGCTAATCTTGCAGGTAGCCCAGCTATCAAAAAGGGAATACTGCAGACC
GTTAAGGTCGTGGATGAACTCGTCAAAGTAATGGGAAGGCATAAGCCCGAGAATATCGTTATCGAGATGGCCCGAGAGAA
CCAAACTACCCAGAAGGGACAGAAGAACAGTAGGGAAAGGATGAAGAGGATTGAAGAGGGTATAAAAGAACTGGGGTCCC
AAATCCTTAAGGAACACCCAGTTGAAAACACCCAGCTTCAGAATGAGAAGCTCTACCTGTACTACCTGCAGAACGGCAGGG
ACATGTACGTGGATCAGGAACTGGACATCAATCGGCTCTCCGACTACGACGTGGATCATATCGTGCCCCAGTCTTTTCTCA
AAGATGATTCTATTGATAATAAAGTGTTGACAAGATCCGATAAAAATAGAGGGAAGAGTGATAACGTCCCCTCAGAAGAAGT
TGTCAAGAAAATGAAAAATTATTGGCGGCAGCTGCTGAACGCCAAACTGATCACACAACGGAAGTTCGATAATCTGACTAA
GGCTGAACGAGGTGGCCTGTCTGAGTTGGATAAAGCCGGCTTCATCAAAAGGCAGCTTGTTGAGACACGCCAGATCACCA
AGCACGTGGCCCAAATTCTCGATTCACGCATGAACACCAAGTACGATGAAAATGACAAACTGATTCGAGAGGTGAAAGTTA
TTACTCTGAAGTCTAAGCTGGTcTCAGATTTCAGAAAGGACTTTCAGTTTTATAAGGTGAGAGAGATCAACAATTACCACCAT
GCGCATGATGCCTACCTGAATGCAGTGGTAGGCACTGCACTTATCAAAAAATATCCCAAGCTTGAATCTGAATTTGTTTACG
GAGACTATAAAGTGTACGATGTTAGGAAAATGATCGCAAAGTCTGAGCAGGAAATAGGCAAGGCCACCGCTAAGTACTTCT
TTTACAGCAATATTATGAATTTTTTCAAGACCGAGATTACACTGGCCAATGGAGAGATTCGGAAGCGACCACTTATCGAAAC
AAACGGAGAAACAGGAGAAATCGTGTGGGACAAGGGTAGGGATTTCGCGACAGTCCGGAAGGTCCTGTCCATGCCGCAG
GTGAACATCGTTAAAAAGACCGAAGTACAGACCGGAGGCTTCTCCAAGGAAAGTATCCTCCCGAAAAGGAACAGCGACAA
GCTGATCGCACGCAAAAAAGATTGGGACCCCAAGAAATACGGCGGATTCGATTCTCCTACAGTCGCTTACAGTGTACTGGT
TGTGGCCAAAGTGGAGAAAGGGAAGTCTAAAAAACTCAAAAGCGTCAAGGAACTGCTGGGCATCACAATCATGGAGCGAT
CAAGCTTCGAAAAAAACCCCATCGACTTTCTCGAGGCGAAAGGATATAAAGAGGTCAAAAAAGACCTCATCATTAAGCTTC
CCAAGTACTCTCTCTTTGAGCTTGAAAACGGCCGGAAACGAATGCTCGCTAGTGCGGGCGAGCTGCAGAAAGGTAACGAG
CTGGCACTGCCCTCTAAATACGTTAATTTCTTGTATCTGGCCAGCCACTATGAAAAGCTCAAAGGGTCtCCCGAAGATAATG
AGCAGAAGCAGCTGTTCGTGGAACAACACAAACACTACCTTGATGAGATCATCGAGCAAATAAGCGAATTCTCCAAAAGAG
TGATCCTCGCCGACGCTAACCTCGATAAGGTGCTTTCTGCTTACAATAAGCACAGGGATAAGCCCATCAGGGAGCAGGCA
GAAAACATTATCCACTTGTTTACTCTGACCAACTTGGGCGCGCCTGCAGCCTTCAAGTACTTCGACACCACCATAGACAGA
AAGCGGTACACCTCTACAAAGGAGGTCCTGGACGCCACACTGATTCATCAGTCAATTACGGGGCTCTATGAAACAAGAATC
GACCTCTCTCAGCTCGGTGGAGACAGCAGGGCTGACCCCAAGAAGAAGAGGAAGGTGTCGCCAGGGATCCGTCGACTTG
ACGCGTTGATATCAACAAGTTTGTACAAAAAAGCAGGCTACAAAGAGGCCAGCGGTTCCGGACGGGCTGACGCATTGG
ACGATTTTGATCTGGATATGCTGGGAAGTGACGCCCTCGATGATTTTGACCTTGACATGCTTGGTTCGGATGCCCTTGAT
GACTTTGACCTCGACATGCTCGGCAGTGACGCCCTTGATGATTTCGACCTGGACATGCTGATTAACTCTAGAAGTTCCG
GATCTCCGAAAAAGAAACGCAAAGTTGGTAGCCAGTACCTGCCCGACACCGACGACCGGCACCGGATCGAGGAAAAG
CGGAAGCGGACCTACGAGACATTCAAGAGCATCATGAAGAAGTCCCCCTTCAGCGGCCCCACCGACCCTAGACCTCCA
CCTAGAAGAATCGCCGTGCCCAGCAGATCCAGCGCCAGCGTGCCAAAACCTGCCCCCCAGCCTTACCCCTTCACCAGC
AGCCTGAGCACCATCAACTACGACGAGTTCCCTACCATGGTGTTCCCCAGCGGCCAGATCTCTCAGGCCTCTGCTCTGG
CTCCAGCCCCTCCTCAGGTGCTGCCTCAGGCTCCTGCTCCTGCACCAGCTCCAGCCATGGTGTCTGCACTGGCTCAGGC
ACCAGCACCCGTGCCTGTGCTGGCTCCTGGACCTCCACAGGCTGTGGCTCCACCAGCCCCTAAACCTACACAGGCCGG
CGAGGGCACACTGTCTGAAGCTCTGCTGCAGCTGCAGTTCGACGACGAGGATCTGGGAGCCCTGCTGGGAAACAGCAC
CGATCCTGCCGTGTTCACCGACCTGGCCAGCGTGGACAACAGCGAGTTCCAGCAGCTGCTGAACCAGGGCATCCCTGT
GGCCCCTCACACCACCGAGCCCATGCTGATGGAATACCCCGAGGCCATCACCCGGCTCGTGACAGGCGCTCAGAGGC
CTCCTGATCCAGCTCCTGCCCCTCTGGGAGCACCAGGCCTGCCTAATGGACTGCTGTCTGGCGACGAGGACTTCAGCTC
TATCGCCGATATGGATTTCTCAGCCTTGCTGGGCTCTGGCAGCGGCAGCCGGGATTCCAGGGAAGGGATGTTTTTGCCG
AAGCCTGAGGCCGGCTCCGCTATTAGTGACGTGTTTGAGGGCCGCGAGGTGTGCCAGCCAAAACGAATCCGGCCATTT
CATCCTCCAGGAAGTCCATGGGCCAACCGCCCACTCCCCGCCAGCCTCGCACCAACACCAACCGGTCCAGTACATGAG
CCAGTCGGGTCACTGACCCCGGCACCAGTCCCTCAGCCACTGGATCCAGCGCCCGCAGTGACTCCCGAGGCCAGTCAC
CTGTTGGAGGATCCCGATGAAGAGACGAGCCAGGCTGTCAAAGCCCTTCGGGAGATGGCCGATACTGTGATTCCCCAG
AAGGAAGAGGCTGCAATCTGTGGCCAAATGGACCTTTCCCATCCGCCCCCAAGGGGCCATCTGGATGAGCTGACAACC
ACACTTGAGTCCATGACCGAGGATCTGAACCTGGACTCACCCCTGACCCCGGAATTGAACGAGATTCTGGATACCTTCC
TGAACGACGAGTGCCTCTTGCATGCCATGCATATCAGCACAGGACTGTCCATCTTCGACACATCTCTGTTTTGA

Cell Culture Transfection and Transduction.

All cells were incubated at 37° C. and 5% CO₂.

C2C12 cells were obtained from the American Tissue Collection Center (ATCC, Manassas, Va.), and grown in growth media (DMEM+glutaMAX+10% FBS). Cells were split with TypLE Express (Invitrogen) every 2-3 days and before reaching 80% confluency, to prevent terminal differentiation. Passage number was kept below 15. For transfection of C2C12 myoblasts, 10⁵ cells were plated per well in a 24-well plate, in 500 μl of growth media. The following day, fresh media was replaced, and 800 ng of total plasmid DNA was transfected with 2.4 μl of Lipofectamine 2000 (Life Technologies). 1:1 mass ratio of vectors encoding Cas9:gRNA(s) was used. Media was replaced with differentiation media (DMEM+glutaMAX+2% donor horse serum) on the 1^{st} and 3^{rd} days post-lipofection.

For differentiation of C2C12 into myotubes, 2×10⁴ cells were plated per well in a 96-well plate, in 100 μl of growth media. At confluency, 1-2 day(s) after plating, media was replaced with fresh differentiation media (DMEM+glutaMAX+2% donor horse serum), and further incubated for 4 days. Fresh differentiation media was replaced before transduction with AAVs. Culture media was replaced with fresh differentiation media 1d after transduction, and cells incubated for stated durations.

The 3×Stop-tdTomato reporter cell line was derived from tail-tip fibroblasts of Ai9 (Madisen, L. et al. A robust and high-throughput Cre reporting and characterization system for the whole mouse brain. Nat Neurosci 13, 133-140, doi:10.1038/nn.2467 (2010)) mouse (JAX 007905), and immortalized with lentiviruses encoding the large SV40 T-antigen (GenTarget Inc, LVP016-Puro). Cells were cultured in DMEM+pyruvate+glutaMAX+10% FBS. Lipofectamine 2000 (Life Technologies) was used for transfection of plasmids, and images were taken 5 days after transfection. For transduction with AAVs, cells were plated at 2×10⁴ per well in a 96-well plate, in 100 μl of growth media. AAV-containing lysates or purified AAVs were applied at confluency of 70-90%. Culture media was replaced with fresh growth media the next day, and cells incubated for stated durations.

The GC-1 spg mouse spermatogonial cell line (CRL-2053) was obtained from ATCC. Cells were cultured and transduced similarly to the 3×Stop-tdTomato cell line, with a Cas9^{N}:Cas9^{C} of 1:1.

Animals.

All animal procedures were approved by the Harvard University Institutional Animal Care and Use Committee.

Ai9 (Madisen, L. et al. A robust and high-throughput Cre reporting and characterization system for the whole mouse brain. Nat Neurosci 13, 133-140, doi:10.1038/nn.2467 (2010)) mice (JAX No. 007905) were used for tdTomato activation, and for systemic AAV9-Cas9-gRNAs and AAV9-GFP-Cre experiments. C57BL/6 male mice were used for in vivo electroporation and intramuscular AAV injections.

All animals were randomly allocated to treatment groups and handled equally.

In vivo electroporation. Animals were anesthetized using isoflurane and injected with 50 μl of 2 mg/ml hyaluronidase (Sigma-Aldrich, H4272) in the tibialis anterior muscle. After 1 hr, plasmids in vehicle (10 mM Tris-HCl pH 8.0) were injected into the muscle, followed by electroporation (Aihara, H. & Miyazaki, J. Gene transfer into muscle by electroporation in vivo. Nature biotechnology 16, 867-870, doi:10.1038/nbt0998-867 (1998)) (10 pulses of 20 ms at 100 V/cm with 100 ms intervals) using an ECM 830 Electro Square Porator (BTX Harvard Apparatus) and a two-needle array.

For conditions with immunosuppression, FK506 (Sigma-Aldrich, F4679) was administered daily at 5 mg/kg (body weight), commencing 1 day before electroporation.

For Cas9-specific T-cell clonotyping and antibody epitope-mapping, both tibialis anterior muscles of 11-week old male C57BL/6 mice were each electroporated with 30 μg of pSMVP-Cas9^{FL}. Control mice were electroporated with 30 μg of plasmid vector control (consisting of the same plasmid with Cas9 coding sequence removed) per muscle. Vehicle electroporations were similarly performed. For intramuscular IL-2 and perforin immunofluorescence staining and centrally nucleated myofiber quantification, 30 μg of DNA minicircles vectors encoding SpCas9^{FL}, 30 μg total of pCRII-U6-gRNA, and/or 15 μg of pCAG-GFP were used as indicated.

Genotyping and Analysis.

C2C12 cells were harvested 4 days post-lipofection, with 100 μl of QuickExtract DNA Extraction Solution (Epicentre) per well of a 24-well plate; and C2C12 myotubes were harvested 7 days post-AAV transduction, with 20 μl of DNA QuickExtract per well of a 96-well plate. Cell lysates were heated at 65° C. for 10 min., 95° C. for 8 min., and stored at −20° C. Each locus was amplified from 0.5 μl of cell culture lysate per 25 μl PCR reaction, for 20-25 cycles.

Bulk tissues were each placed in 100 μl of QuickExtract DNA Extraction Solution, and heated at 65° C. for 15 min., 95° C. for 10 min. 0.5 μl of lysate was used per 25 μl PCR reaction, and thermocycled for 25 cycles.

For barcoding for deep sequencing, 1 μl of each unpurified PCR reaction was added to 20 μl of barcoding PCR reaction, and thermocycled [95° C. for 3 min., and 10 cycles of (95° C. for 10 s, 72° C. for 65 s)]. Amplicons were pooled, the whole sequencing library purified with self-made SPRI beads (9% PEG final concentration), and sequenced on a Miseq (Illumina) for 2×251 cycles. FASTQ were analyzed with BLAT (Kent, W. J. BLAT—the BLAST-like alignment tool. Genome Res 12, 656-664, doi:10.1101/gr.229202 (2002)) (with parameters —t=dna —q=dna —tileSize=11 —stepSize=5 —oneOff=1 —repMatch=10000000 —minMatch=4 —minIdentity=90 —maxGap=3 —noHead) and post-alignment analyses performed with MATLAB (MathWorks). Alignments due to primer dimers were excluded by filtering off sequence alignments that did not extend >2 bp into the loci from the locus-specific primers. To minimize the impact of sequencing errors, conservative variant calling was performed by ignoring base substitutions, and calling only variants that overlap with a ±30 bp window from the designated Cas9-gRNA cut sites. Negative controls were equally analyzed for baseline sequencing error rates, to which statistical tests were performed against.

Off-target sites for Mstn gRNAs were predicted using the online CRISPR Design Tool (Hsu, P. D. et al. DNA targeting specificity of RNA-guided Cas9 nucleases. Nature biotechnology 31, 827-832, doi:10.1038/nbt.2647 (2013)) (world wide website crispr.mit.edu). Off-target sites were ranked by number of mismatches to the on-target sequence, and deep sequencing performed on top hits. Sequencing reads were analyzed equally between experimental samples (AAV9-Cas9-gRNAs^{M3→M4}) and control samples (AAV9-Cas9-gRNAs^{TdL+TdR}) using BLAT. Variant calls were performed for insertions and deletions that lie within a ±15 bp window from potential off-target cut-sites.

Quantitative Reverse-Transcription PCR (qRT-PCR) for Gene Expression.

Cells were processed with Taqman Cells-to-Ct kits (Thermo Fisher Scientific #4399002) as per manufacturer's instructions, with the modification that each qRT-PCR reaction was scaled down to 25 μl. Taqman hydrolysis probes (Thermo Fisher Scientific) used: PD-L1 (Mm00452054_m1), FST (Mm00514982_m1), CD47 (Mm00495011_m1), and house-keeping gene Abl1 (Mm00802029_m1). Gene expression from targeted genes were normalized to that of Abl1 (ΔCt) and fold-changes were calculated against AAV-Cas9^{C}-VPR-only controls (no-gRNA) (2^{-ΔΔct}). Basal gene expression percentiles for C2C12 myotubes and GC-1 spermatogonial cells (type B. spermatogonia) were retrieved from the Gene Expression Omnibus (GEO) repository (GDS2412 and GDS2390 respectively).

Total RNA from skeletal muscle tissues was extracted via TRIzol. Reverse transcription was conducted with High-Capacity cDNA Reverse Transcription Kit (Applied Biosystems #4368814), and 5 μl of each reaction was used for qRT-PCR in 1× FastStart Essential DNA Probes Master (Roche #06402682001). Gene expression from targeted genes were normalized to that of Abl1 (ΔCt) and fold-changes were calculated against AAV9-turboRFP-only controls (2^{-ΔΔct}).

AAV Administration in Mice.

AAV and control experiments were conducted in a randomized and double-blind fashion. The allocation code was unblinded only after analyses were completed. AAV9-Cas9-gRNAs injections utilized AAV9-Cas9N-gRNA:AAV9-Cas9^{C}-P2A-turboGFP ratio of 1:1.

3-day old neonates were each intraperitoneally injected with 4E12, 5E11, or 2.5E11 vector genomes (vg) of total AAV9. Vector volumes were kept at 100 μl. Animals were euthanized via CO₂ asphyxia and cervical dislocation 3 weeks following injections. For AAV9-GFP and AAV9-mCherry co-transduction experiments, animals were euthanized 9 days after injection. For qPCR and deep sequencing of whole tissues, samples were taken from the heart body wall, liver, gastrocnemius muscle, olfactory bulb, ovary, testis, and diaphragm.

AAV9-Cas9-VPR-gRNAs were intramuscularly injected at AAV9-Cas9^{N}-gRNAs:AAV9-Cas9^{C}-VPR ratio of 1:1, at a total of 4E12 vg. To demarcate transduced tissues, 1E11 of AAV9-turboRFP was coadministered in the same mix. Controls mice were injected with 1E11 of AAV9-turboRFP only, with the final injection mix at the same volume.

For determining AAV9- and Cas9-specific T-cell clonotypes and antibody epitopes, both tibialis anterior muscles of 11-week old male C57BL/6 mice were each injected with AAV9-Cas9^{N} and AAV9-Cas9^{C} (2E12 vg each). For control mice, 4E12 of AAV9 vector control (consisting of the same AAV genome with the Cas9 coding sequence removed) was injected per muscle. Vehicle (1×PBS+35 mM NaCl) injections were similarly performed. For IL-2 and perforin immunofluorescence staining and centrally nucleated myofiber quantification, muscles were injected with AAV9-Cas9-VPR-gRNAs at 4E12 vg and AAV9-turboRFP at 1E11 vg, while control mouse muscles were injected with the same volume of vehicle and AAV9-turboRFP at 1E11 vg.

Quantitative PCR (qPCR) for AAV Genomic Copies in Tissues.

Each qPCR reaction consists of 1× FastStart Essential DNA Probes Master (Roche #06402682001), 100 nM of each hydrolysis probe (against the AAV ITR and the mouse Acvr2b locus), 340 nM of AAV ITR reverse primer, 100 nM each for all other forward and reverse primers, and 2.5 μl of input tissue lysate. A mastermix was first constituted before splitting 22.5 μl into each well, after which tissue lysates were added. Thermocycling conditions were: [95° C. 15 min.; 40 cycles of (95° C. 1 min., 60° C. 1 min.)]. FAM and HEX fluorescence were taken every cycle. AAV genomic copies per mouse diploid genome were calculated against standard curves. For each tissue sample, two repeated samplings were performed for qPCR and deep-sequencing, all on separate days, and the means plotted with s.e.m. qPCR false positive rate were calculated similarly from two vehicle-injected negative control mice, to which statistical tests were performed against.

Cas9 Re-Stimulation and TCR-β Repertoire Sequencing.

Lymphocytes were isolated from 2× inguinal lymph nodes and 1× popliteal lymph node per bilaterally injected mouse. Lymph nodes were scored, and incubated for 30 min. in RPMI+1 mg/ml collagenase at RT. Lymphocytes were released by meshing through 70 μm nylon sieves, washed twice with 1×PBS+5 mM EDTA, and resuspended in 500 μl of growth media (RPMI 1640+10% FBS+1×Pen/Strep/AmphoB+50 μM 2-βME). Cell counting was performed on a Countess device (Life Technologies). For Cas9 restimulation experiments, 2.5 μg of recombinant Cas9 (NEB) was incubated with >10⁶ cells in 500 μl of growth media for 3 days. Non-restimulated wells were conducted in parallel without Cas9. Cellular RNA was extracted using QIAshredder and RNeasy micro (Qiagen). Reverse transcription was performed with SMARTscribe (Clontech), using SMARTNNN template-switching adapter as described (Mamedov, I. Z. et al. Preparing unbiased T-cell receptor and antibody cDNA libraries for the deep next generation sequencing profiling. Frontiers in immunology 4, 456, doi:10.3389/fimmu.2013.00456 (2013)). KAPA HiFi polymerase was used for PCR. Individual RNA molecules were counted based on Unique Molecular Identifiers using MIGEC (Shugay, M. et al. Towards error-free profiling of immune repertoires. Nature methods 11, 653-655, doi:10.1038/nmeth.2960 (2014)), aligned with MIXCR (Bolotin, D. A. et al. MiXCR: software for comprehensive adaptive immunity profiling. Nature methods 12, 380-381, doi:10.1038/nmeth.3364 (2015)), and post-analysis performed with MATLAB (MathWorks). Morisita-Horn indices per exposure condition were calculated by pairwise comparisons among 4 mice (2 animals from electroporation dataset and 2 animals from AAV dataset).

Fluorescent Immunoassay.

To determine antibody specificity and class-switching, serum levels of AAV9-specific IgM, IgG, and IgG2a from AAV9-treated mice were compared to that from vehicle-injected control mice. AAV9 viruses (1E9 vg) were coated on each well of a 96-well PVDF MaxiSorp plate for 1 hr in 1×TBST, followed by 1 hr of blocking in 1×TBST+3% BSA. After 3 washes with 1×TBST, 1:100 diluted mouse serum was applied at 25 μl per well, for 1 hr. After 3 washes with 1×TBST, 1:200 diluted anti-mouse secondary antibodies were added [goat anti-mouse IgG-CF633 (Biotium 20120), goat anti-mouse IgG2a-CF594 (Biotium 20259) and goat anti-mouse IgM-Dy550 (Pierce PISA510151)], and incubated for 1 hr. Wells were then washed 5× with 1×TBST, and fluorescence readings in 100 μl of 1×TBS were taken via a plate reader. All steps were conducted at RT. To account for autofluorescence from sera, fluorescence readings were normalized against that from wells treated similarly except for the exclusion of secondary antibodies.

Epitope Mapping by M13 Phage Display.

M13KE genome was amplified by PCR, with one end terminating with the pIII peptidase cleavage signal, and the other end terminating with a 4×Gly linker followed by the mature pIII. Cas9 and AAV9 VP1 capsid coding sequence PCR products were each randomly fragmented with NEBNext dsDNA Fragmentase until about 50-300 bp. Purified fragments were treated with NEBNext End-Repair Module. After DNA purification, fragments were blunted ligated into the M13KE PCR product overnight at 16° C. The entire ligation reaction was purified, and transformed into ER2738 (Lucigen), at 200 ng per 25 μl of bacteria, with electroporation conditions of 10 μF, 600Ω, and 1.8 kV. After 30 min. recovery in SOC media, the culture was amplified by combining with 20 ml of early-log ER2738 culture. After 4 hrs, the culture supernatant was collected, and incubated to a final concentration of 3.33% PEG-8000 and 417 mM of NaCl, overnight at 4° C. M13 phage was pelleted, and resuspended in 2 ml of TBS. Phage titers were determined by LB/IPTG/X-gal blue-white plague counting, averaging >1E11 pfu/μl. For Ig:phage pull-down, 20 μl of each phage library was incubated with 5 μl of mouse serum or titrated amount of purified antibody controls [7A9 (Novus Bio), Guide-IT (Clontech), bG15 (Santa Cruz), bS18 (Santa Cruz), bD20 (Santa Cruz), non-binding mouse IgG isotype control (Santa Cruz)], and made up to 50 μl with TBST, for 1 hr at RT. For each reaction, 25 μl of Protein A/G magnetic beads (Millipore PureProteome) was first washed twice with TBST, resuspended to 10 μl, and added to the reaction for additional 30 min. incubation. The beads were then washed 5× with TBST, and captured Ig:phage eluted with 100 μl of 200 mM glycine-HCl, pH 2.2, 1 mg/ml BSA for 8 min. The eluant was neutralized with 15 μl of 1 M Tris-HCl, pH 8.5. 5 μl of captured phage display eluant was used per PCR reaction, with 20 cycles of spacer amplification and 10 cycles of barcoding, and sequenced on a Miseq (Illumina). Each serum sample was processed for technical replication on separate days. Differential binding of phage was determined using DESeq2 (Love, M. I., Huber, W. & Anders, S. Moderated estimation of fold change and dispersion for RNA-seq data with DESeq2. Genome biology 15, 550, doi:10.1186/s13059-014-0550-8 (2014)) in R (Team, R. C. (ISBN 3-900051-07-0, 2014)), with all Cas9-unexposed (n=16 animals) or AAV-unexposed (n=16 animals) samples as appropriate controls for non-specific binding. Alignments and further analyses were performed with MATLAB (MathWorks). Visualization of epitopes on Cas9 (PBD ID: 4CMP, chain A) and AAV9 VP3 (PBD ID: 3UX1) structures was conducted with Pymol. Phenotypic data of double-alanine AAV9 mutants were obtained from Adachi, K., Enoki, T., Kawano, Y., Veraz, M. & Nakai, H. Drawing a high-resolution functional map of adeno-associated virus capsid by massively parallel sequencing. Nat Commun 5, 3075, doi:10.1038/ncomms4075 (2014), with mutant viral blood persistency calculated as the difference in blood viral levels 72 hr. and 10 min. post-injection (both normalized to that of wildtype AAV9, with 0 denoting wildtype phenotype, and negative values denoting loss of blood persistency). Mutant tropism represented by ‘Phenotypic Difference’ values as described in Adachi, K., Enoki, T., Kawano, Y., Veraz, M. & Nakai, H. Drawing a high-resolution functional map of adeno-associated virus capsid by massively parallel sequencing. Nat Commun 5, 3075, doi:10.1038/ncomms4075 (2014). Solvent-accessibility surface area (sasa) ratios for AAV9 capsid (PDB ID: 3UX1) were first calculated as described in Mandell, D. J. et al. Biocontainment of genetically modified organisms by synthetic protein design. Nature 518, 55-60, doi:10.1038/nature14121 (2015), and the final sasa ratio per residue calculated as the mean from a ±5 bp sliding window centered on the residue.

Total mRNA-Sequencing.

1 μg of TRIzol-extracted RNA from muscle tissues were enriched for polyA-tailed mRNA and processed with NEBNext Ultra Directional RNA Library Prep Kit (New England Biolabs), followed by sequencing on a NextSeq sequencer (Illumina), giving 30 million reads per sample. Reads were aligned to the mm10 reference genome and FPKM quantified with the Cufflinks workflow (Trapnell, C. et al. Transcript assembly and quantification by RNA-Seq reveals unannotated transcripts and isoform switching during cell differentiation. Nature biotechnology 28, 511-515, doi:10.1038/nbt.1621 (2010)), with differential expression tested with Cuffdiff (Trapnell, C. et al. Differential analysis of gene regulation at transcript resolution with RNA-seq. Nature biotechnology 31, 46-53, doi:10.1038/nbt.2450 (2013)). GO-terms network was visualized with ClueGO (Bindea, G. et al. ClueGO: a Cytoscape plug-in to decipher functionally grouped gene ontology and pathway annotation networks. Bioinformatics 25, 1091-1093, doi:10.1093/bioinformatics/btp101 (2009)) Cytoscape (Shannon, P. et al. Cytoscape: a software environment for integrated models of biomolecular interaction networks. Genome Res 13, 2498-2504, doi:10.1101/gr.1239303 (2003)) plug-in.

Histology and Immunofluorescence Staining.

Mouse organs and tissue samples were dissected, fixed in 4% paraformaldehyde in 1×DPBS for 1.5 hr, followed by 3×5 min. washes with 1×DPBS. Samples were immersed in 30% sucrose until submersion, embedded in O.C.T. compound (Tissue-Tek), frozen in liquid-nitrogen-cold isopentane, and cryosectioned on a Microm HM550 (Thermo Scientific). Skeletal muscles were sectioned to a thickness of 12 μm, while the liver and heart were sectioned at 20 μm.

For immunofluorescence, TA muscle sections were blocked in 1×PBST+3% BSA for 1 hr at RT, immunostained with primary antibodies at RT for 1 hr, followed by 3× washes with PBS/T. Slides were then incubated with secondary antibodies at RT for 1 hr, followed by 3× washes with PBS/T. Anti-mouse IL-2 and perforin antibodies were used at 1:100 (Santa Cruz sc-7896 and sc-9105 respectively), followed by 1:200 of secondary anti-rabbit CF633 (Biotium). Immunostained slides were mounted with mounting media containing DAPI (Vector Laboratories, H1500).

Western Blot.

Muscles were harvested 2 weeks after AAV injections or plasmid electroporation. ˜10 mm³ tissue clippings were flash-frozen in liquid nitrogen, followed by lysis in 300-500 μl of T-PER Tissue Protein Extraction Solution (Thermo Scientific) supplemented with 1× Complete Protease Inhibitor (Roche), and homogenized in gentleMACS M tubes (Miltenyi Biotec). 10-15 μl of each tissue lysate was ran on 8% Bolt Bis-Tris Plus gels (Life Technologies) in 1× Bolt MOPS SDS running buffer at 165 V for 50 min. Protein transfer was performed with iBlot (Life Technologies) onto PVDF membranes, using program 3 for 13 min. Western blots were conducted with 1:200 of anti-Cas9 Guide-IT polyclonal antibody (Clontech 632607), 1:400 of anti-GAPDH polyclonal antibody (Santa Cruz sc-25778), and 1:2500 of anti-rabbit IgG-HRP secondary antibody (Santa Cruz sc-2004), using an iBind device (Life Technologies). Stained membranes were developed with SuperSignal West Femto Maximum Sensitivity Substrate (Thermo Scientific) and imaged on Chemidoc MP (Bio-Rad). Band intensities were quantified with ImageJ (NIH).

Immunosuppression.

FK506 was dissolved in 100% DMSO, and the stock solution further diluted 1:100 in vehicle for final concentrations of 1% DMSO, 10% Cremophor (Sigma-Aldrich, C5135), and 1×PBS. Mice were injected daily with 5 mg/kg (body weight) of FK506, with the first injection commencing 1 day before in vivo electroporation.

30 μg of minicircle-SMVP-Cas9 was injected for Cas9-only injections, 15 μg of pCAG-GFP for GFP-only injections, and 30 μg of minicircle-SMVP-Cas9 and 15 μg of pCAG-GFP for Cas9+GFP injections. 4 mice were injected per condition.

Imaging and Analyses.

Confocal images were taken using a Zeiss LSM780 inverted microscope. For live cell-imaging, each image consists of 3× z-stacks (7 μm intervals) and 2×2 tiles. For muscle sections, 3× z-stacks (7 μm intervals) were used. For liver and heart sections, 4× z-stacks (10 μm intervals) were used. For imaging of all tissue sections, tiling to cover entire samples were used, followed by stitching. Stacked fluorescence images were projected by maximum intensity with Zen 2011 (Carl Zeiss).

Epifluorescence images were taken with an Axio Observer D1 (Carl Zeiss) or Axio Observer Z1 (Carl Zeiss).

For co-transduction analysis after AAV9-GFP and AAV9-mCherry administration, pixels that contain both GFP and mCherry fluorescence intensities above the background thresholds were identified, and the lower intensity values from either channel were used to populate a merged image. All other pixels in the merged image were set to null.

Whole organ images were taken on a SMZ1500 (Nikon) fluorescence dissection stereomicroscope equipped with a SPOT RT3 camera (Diagnostic Instruments), for an imaging area of 16 mm by 12 mm, with 3 s exposure for the liver and 4 s exposure for the heart, muscle, brain and gonads. All images were acquired with a gain setting of 8 using the SPOT imaging software (Diagnostic Instruments, Sterling Heights, Mich.). Images for each organ were inverted and thresholded equally across animals.

Images were analyzed with ImageJ (NIH), CellProfiler (Carpenter, A. E. et al. CellProfiler: image analysis software for identifying and quantifying cell phenotypes. Genome biology 7, R100, doi:10.1186/gb-2006-7-10-r100 (2006)) and MATLAB (MathWorks).

### Cas9 Orthologs and Applications of AAV-Split-Cas9

### Targeting Range of Cas9 Orthologs (FIG. 1A)

Cas9 orthologs, such as those from S. aureus (Sa) (Ran, F. A. et al. In vivo genome editing using Staphylococcus aureus Cas9. Nature 520, 186-191, doi:10.1038/nature14299 (2015)), S. thermophilus (St1) (Esvelt, K. M. et al. Orthogonal Cas9 proteins for RNA-guided gene regulation and editing. Nature methods 10, 1116-1121, doi:10.1038/nmeth.2681 (2013)) and N. meningitides (Nm) (Esvelt, K. M. et al. Orthogonal Cas9 proteins for RNA-guided gene regulation and editing. Nature methods 10, 1116-1121, doi: 10.1038/nmeth.2681 (2013)), present exciting and complementary ways to manipulate the genome. However, with more restrictive PAM requirements, these orthologs are biologically unable to recognize the large spectrum of genomic sites accessible by SpCas9. This diminishes the key attractiveness of CRISPR-Cas9 in facile DNA-addressing. PAM requirements can be altered by artificially evolving Cas9s, which significantly broadens the targeting ranges (Kleinstiver, B. P. et al. Engineered CRISPR-Cas9 nucleases with altered PAM specificities. Nature 523, 481-485, doi:10.1038/nature14592 (2015); Kleinstiver, B. P. et al. Broadening the targeting range of Staphylococcus aureus CRISPR-Cas9 by modifying PAM recognition. Nature biotechnology, doi:10.1038/nbt.3404 (2015)). Considering canonical, non-canonical and altered PAMs, SpCas9 requires an NRG, NGR, or NGCG PAM, while SaCas9 requires an NNGRR or NNNRRT PAM (these are referred to as Sp* and Sa* respectively in FIG. 1A). We note that this is an underestimate of the current Sp*Cas9 targeting range, because SpCas9 fused with DNA-binding domains allows targeting of the NGC PAM (Bolukbasi, M. F. et al. DNA-binding-domain fusions enhance the targeting range and precision of Cas9. Nature methods 12, 1150-1156, doi:10.1038/nmeth.3624 (2015)). The relaxed NGC PAM is not included in our analysis, in the spirit to maintain conservative comparison in the absence of such engineering conducted with any of the other Cas9 orthologs. We also note that in the context of AAV delivery, in order to harness the SpCas9-fusion variants for enhancing targeting range and specificity, a split-Cas9 approach would be necessary. Together, these engineering efforts further increase the gap between Sp* and Sa* Cas9s against the other orthologs, with Sp* Cas9 retaining the broadest targeting range.

The advantage of a relaxed PAM is exponential when multiple sites are targeted within a genome, where the probability of finding multiple suitable sites is a product of the PAM densities. A useful application of multiplex CRISPR-Cas9 would be to generate genomic excisions, as we apply here. The PAM density also dictates the feasibility of widely used CRISPR-Cas9 tools. For example, specificity of CRISPR-Cas9 gene-targeting is significantly increased with the use of paired Cas9-nickases (Mali, P. et al. CAS9 transcriptional activators for target specificity screening and paired nickases for cooperative genome engineering. Nature biotechnology 31, 833-838, doi:10.1038/nbt.2675 (2013); Ran, F. A. et al. Double nicking by RNA-guided CRISPR Cas9 for enhanced genome editing specificity. Cell 154, 1380-1389, doi:10.1016/j.cell.2013.08.021 (2013)) or dCas9-FokI (Tsai, S. Q. et al. Dimeric CRISPR RNA-guided FokI nucleases for highly specific genome editing. Nature biotechnology 32, 569-576, doi:10.1038/nbt.2908 (2014); Tsai, S. Q. et al. Dimeric CRISPR RNA-guided FokI nucleases for highly specific genome editing. Nature biotechnology 32, 569-576, doi:10.1038/nbt.2908 (2014)), which requires proximal binding of two Cas9-gRNA complexes to effect double-strand breaks. Importantly, these approaches operate on the basis that endonucleolytic activity is constituted only when both Cas9-gRNA complexes are within a certain molecular distance from each other (<100 bp for offset nicking with Cas9-nickases; 15 bp or 25 bp for dCas9-FokI). Existence of two Cas9-gRNA target sites within these specific distances is hence necessary for function. The numbers of human (FIG. 1A) and mouse exonic sites that can be targeted with these specificity-enhancing approaches are orders of magnitude higher for SpCas9, compared to the other orthologs.

### Activity of Cas9 Orthologs

While SpCas9 has been successfully employed to target a myriad of genomic sites across a broad spectrum of species, it is now well-documented that individual gRNAs can exhibit variable targeting efficiencies. Likewise, there are hints that the other orthologs exhibit similar variability. For example, in the first demonstration of SaCas9 for gene-editing in the liver (Ran, F. A. et al. In vivo genome editing using Staphylococcus aureus Cas9. Nature 520, 186-191, doi:10.1038/nature14299 (2015)), Pcsk9 was targeted at ˜40%, while Apob at 0% to 8.9%.

It would hence be illuminating to compare activities of various orthologs on a global scale, to determine if putative target sites can be edited, and how efficiently if so. For example, St1Cas9 generally underperformed SpCas9 across >1000 tested gRNAs (Chari, R., Mali, P., Moosburner, M. & Church, G. M. Unraveling CRISPR-Cas9 genome engineering parameters via a library-on-library approach. Nature methods 12, 823-826, doi:10.1038/nmeth.3473 (2015)). Comprehensive comparisons between CRISPR-Cas9s and CRISPR-Cpf1s are anticipated for these highly enticing systems.

AAV-Split-Cas9 Allows Domain Fusions and Compatibility with Self-Complementary AAVs

Split-Cas9 shortens the coding sequences significantly below all known Cas9 orthologs, which liberates the severely limited AAV capacity for the many exciting applications with CRISPR-Cas9. Since Cas9 and Cpf1 proteins generally adopt a bi-lobed structure (Nishimasu, H. et al. Crystal structure of Cas9 in complex with guide RNA and target DNA. Cell 156, 935-949, doi:10.1016/j.cell.2014.02.001 (2014); Jinek, M. et al. Structures of Cas9 endonucleases reveal RNA-mediated conformational activation. Science 343, 1247997, doi:10.1126/science.1247997 (2014); Nishimasu, H. et al. Crystal Structure of Staphylococcus aureus Cas9. Cell 162, 1113-1126, doi:10.1016/j.cell.2015.08.007 (2015); Hirano, H. et al. Structure and Engineering of Francisella novicida Cas9. Cell 164, 950-961, doi:10.1016/j.cell.2016.01.039 (2016)), it is foreseen this split-reconstitution engineering framework to be generically applicable, even for orthologs yet to be characterized, which would likely be necessary when novel Cas9 and Cpf1 domain fusions are to be made (such as with more specific and efficient nucleolytic domains, epigenetic effectors, protein complex recruiters, inducible domains, nucleotide base editors, and the like). These protein domains generally range in the hundreds to thousands of base-pairs. For comparison, the all-in-one AAV-SpCas9^{FL}-gRNA (Senis, E. et al. CRISPR/Cas9-mediated genome engineering: an adeno-associated viral (AAV) vector toolbox. Biotechnology journal 9, 1402-1412, doi:10.1002/biot.201400046 (2014)) and AAV-SaCas9^{FL}-gRNA (Ran, F. A. et al. In vivo genome editing using Staphylococcus aureus Cas9. Nature 520, 186-191, doi:10.1038/nature14299 (2015)) designs as described are >4.8 kb, and would not be able to accommodate additional elements in their current forms.

Furthermore, we show here that genome-editing frequency is highly dependent on delivery efficiency. The split-reconstitution paradigm could also grant compatibility of Cas9s and Cpf1s with self-complementary AAV (payload limit of 2.4-3.3 kb), which confers transduction efficiency often superior to conventional single-stranded AAVs (McCarty, D. M. Self-complementary AAV vectors; advances and applications. Mol Ther 16, 1648-1656, doi:10.1038/mt.2008.171 (2008)).

### Split-Cpf1 and Split-Ago

On a similar design principle as our approach, splitting Cpf1 orthologs at sites that maximize the likelihood for proper folding of each lobe might be attempted. Structures for Cpf1 proteins have been determined (Dong D, Ren K, Qiu X, Zheng J, Guo M, Guan X, Liu H, Li N, Zhang B, Yang D, Ma C, Wang S, Wu D, Ma Y, Fan S, Wang J, Gao N, Huang Z, The crystal structure of Cpf1 in complex with CRISPR RNA, Nature, 2016 April 28; 532(7600):522-6; Yamano T, Nishimasu H, Zetsche B, Hirano H, Slaymaker I M, Li Y, Fedorova I, Nakane T, Makarova K S, Koonin E V, Ishitani R, Zhang F, Nureki O, Crystal Structure of Cpf1 in Complex with Guide RNA and Target DNA, Cell, 2016 May 5; 165(4):949-62). From the LbCpf1 structure (PDB ID: 5ID6), the less structured linkers at V280-E292, Q513-K520, and N803-F810 might be appropriate split-sites. From the AsCpf1 structure (PDB ID: 5B43), the less structured linkers between S311-S325, T522-K530, M795-E804, and N878-K887 might be appropriate split-sites. Because both structures were determined from nucleic-acid(s) bound Cpf1 proteins, free Cpf1 apoenzyme structures might reveal more of such potential split-sites.

Similarly, previously determined protein structures show that Argonaute proteins are also bilobal, and hence would be appropriate for the current approach. The long prokaryotic argonautes comprise of an N-terminal PAZ lobe and a C-terminal PIWI lobe, connected by Linker 2 (L2) (See Swarts, D. C. et al. The evolutionary journey of Argonaute proteins. Nature structural & molecular biology 21, 743-753, doi:10.1038/nsmb.2879 (2014); Song, J. J., Smith, S. K., Hannon, G. J. & Joshua-Tor, L. Crystal structure of Argonaute and its implications for RISC slicer activity. Science 305, 1434-1437, doi:10.1126/science.1102514 (2004); Wang, Y., Sheng, G., Juranek, S., Tuschl, T. & Patel, D. J. Structure of the guide-strand-containing argonaute silencing complex. Nature 456, 209-213, doi:10.1038/nature07315 (2008); Sheng, G. et al. Structure-based cleavage mechanism of Thermus thermophilus Argonaute DNA guide strand-mediated DNA target cleavage. Proceedings of the National Academy of Sciences of the United States of America 111, 652-657, doi:10.1073/pnas.1321032111 (2014)). This conserved bilobal architecture suggests that the L2 domain is an appropriate split-site. For PfAgo (PDB: 1U04), the L2 domain lies E276-R363. Within the L2, Q347-L356 is less structured and might be most preferred. Based on the PfAgo structure, the less structured region at Y413-E443 might also be an appropriate split-site. For TtAgo (PDB: 3DLH, 4N47), the L2 domain lies E272-F338. Based on the TtAgo structure, D269-W283, R315-L321, and T504-P515 are less structured regions, and might be most preferred. NgAgo structure has not been determined, but based on the high structural conservation among orthologs between species and across prokaryotes to eukaryotes, a similar bilobal protein structure is likely. From homology alignment of NgAgo with PfAgo and TtAgo using HHPred (Soding, J., Biegert, A. & Lupas, A. N. The HHpred interactive server for protein homology detection and structure prediction. Nucleic Acids Res 33, W244-248, doi:10.1093/nar/gki408 (2005)), Q417-A438, Y481-T502, and S696-Q707 are potential split-sites.

### AAV-Cas9-gRNA for Homologous Recombination (HR)

The significantly increased space granted by AAV-split-Cas9 and evidence of efficient AAV co-transduction are particularly relevant for applications demanding template-directed HR. To accomplish HR, donor DNA templates have to be co-delivered into the cell with Cas9-gRNA. Even when using the smaller Cas9 orthologs (˜3.3 kb), the incorporation of viral ITRs (0.3 kb), generic transcriptional regulators (˜0.8 kb) and a single polIII promoter-gRNA cassette (0.4 kb) would already push the payload (˜4.8 kb) to the limit of AAV capacity, precluding the incorporation of a HR donor sequence. This implies that to accomplish HR-directed genome-editing, employing dual AAVs would be necessary (Yang, Y. et al. A dual AAV system enables the Cas9-mediated correction of a metabolic liver disease in newborn mice. Nature biotechnology, doi:10.1038/nbt.3469 (2016)), in line with the approach undertaken in this study.

Secondly, both non-homologous end-joining (NHEJ) and HR are processes downstream of dsDNA cleavage, and editing frequencies via either mechanism would depend on and reflect the extent of Cas9-mediated dsDNA cleavage. Hence, editing via NHEJ could serve as a first proxy for potential HR. We characterize the first steps in this process, showing the direct dependence of NHEJ frequency with delivery efficiency across organs. This suggests that HR efficiency would likely also depend on delivery efficiency. Subsequent investigations into developmental stage, cell states and types, and donor sequence properties will be necessary to pinpoint and optimize the parameters underlying competence for HR following systemic delivery of Cas9-gRNA.

### Tissue-Specificity and Small-Molecule Regulation of AAV-Cas9-gRNA

In this study, we demonstrate use of an excision-dependent fluorescence reporter mouse line (Madisen, L. et al. A robust and high-throughput Cre reporting and characterization system for the whole mouse brain. Nat Neurosci 13, 133-140, doi:10.1038/nn.2467 (2010)) for detecting Cas9-gRNA activity in situ. Demarcation of AAV9-Cas9-gRNA biodistribution revealed edited cells across multiple tissue types and organs, enabled by the robustness of AAV9 for systemic delivery. On the other hand, this wide viral spread urges that careful monitoring and confinement of AAV-Cas9-gRNA would be prudent. Enticingly, the dual-AAVs format offers multi-tiered safeguards to restrict Cas9-gRNA activity to specific tissues of interest. The ability to use independent transcriptional and translational elements within the two AAVs would enable stricter tissue-specific regulation, such as by intersecting two or more tissue-specific elements. In addition, using independent AAV serotypes for each split-half and gRNA(s) would further confine Cas9-gRNA function to tissues where tropisms overlap Enhancing tissue-level specificity complements the increased genome-level specificity that has been demonstrated with Cas9 engineering (Mali, P. et al. CAS9 transcriptional activators for target specificity screening and paired nickases for cooperative genome engineering. Nature biotechnology 31, 833-838, doi:10.1038/nbt.2675 (2013); Ran, F. A. et al. Double nicking by RNA-guided CRISPR Cas9 for enhanced genome editing specificity. Cell 154, 1380-1389, doi:10.1016/j.cell.2013.08.021 (2013); Tsai, S. Q. et al. Dimeric CRISPR RNA-guided FokI nucleases for highly specific genome editing. Nature biotechnology 32, 569-576, doi:10.1038/nbt.2908 (2014); Guilinger, J. P., Thompson, D. B. & Liu, D. R. Fusion of catalytically inactive Cas9 to FokI nuclease improves the specificity of genome modification. Nature biotechnology 32, 577-582, doi:10.1038/nbt.2909 (2014); Slaymaker, I. M. et al. Rationally engineered Cas9 nucleases with improved specificity. Science, doi:10.1126/science.aad5227 (2015)). For example, intein insertions can render full-length SpCas9 conditionally inactive/active in response to small molecules, thereby also increasing genomic target-specificity (Davis, K. M., Pattanayak, V., Thompson, D. B., Zuris, J. A. & Liu, D. R. Small molecule-triggered Cas9 protein with improved genome-editing specificity. Nat Chem Biol 11, 316-318, doi:10.1038/nchembio.1793 (2015)). However, none of the 15 tested intein-inserted Cas9 variants retained full Cas9^{FL} activity, potentially due to disruption of the Cas9 structure; furthermore, the coding sequences of these variants (5.4 kb) exceed the AAV payload limitation. To capitalize on the increased targeting specificity of small-molecule inducible Cas9, it is foreseen that combining inducible split-inteins (Mootz, H. D., Blum, E. S., Tyszkiewicz, A. B. & Muir, T. W. Conditional protein splicing: a new tool to control protein structure and function in vitro and in vivo. J Am Chem Soc 125, 10561-10569, doi:10.1021/ja0362813 (2003)) and structure-guided engineered AAV-split-Cas9 (this study) would confer exquisite functional regulation in vivo.

1. A method of altering a target nucleic acid in a cell comprising
providing to the cell a first nucleic acid encoding a first portion of a Cas9 protein and a guide RNA (gRNA),
providing to the cell a second nucleic acid encoding a second portion of the Cas9 protein and optionally a transcriptional regulator,
wherein the cell expresses the first portion of the Cas9 protein, the gRNA and the second portion of the Cas9 protein or the second portion of the Cas9 and the transcriptional regulator fusion protein,
wherein the first portion of the Cas9 protein and the second portion of the Cas9 protein, or the first portion of the Cas9 protein and the second portion of the Cas9 and the transcriptional regulator fusion protein are joined together to form the Cas9 protein or the Cas9 fusion protein, and
wherein the gRNA and the Cas9 protein, or the gRNA and the Cas9 fusion protein form a co-localization complex with the target nucleic acid and alter the expression of the target nucleic acid.

2. The method of claim 1, wherein the first nucleic acid encodes a first portion of the Cas9 protein having a N-split-intein RmaIntN and wherein the second nucleic acid encodes a second portion of the Cas9 protein having a C-split-intein RmaIntC and wherein the first portion of the Cas9 protein and the second portion of the Cas9 protein are joined together to form the Cas9 protein.

3. The method of claim 1, wherein the first portion of the Cas9 protein is the N-terminal lobe of the Cas9 protein up to amino acid V713 and the second portion of the Cas9 protein is the C-terminal lobe of the Cas9 protein beginning at D714.

4. The method of claim 3, wherein the gRNA having a truncated spacer sequence guides the Cas9 protein or the Cas9 fusion protein to the target nucleic acid and regulate the expression of the target nucleic acid without cleaving the target nucleic acid.

5. The method of claim 1, wherein the first nucleic acid and the second nucleic acid are delivered to the cell by separate vectors.

6. The method of claim 1, wherein the vector is adeno-associated virus.

7. (canceled)

8. (canceled)

9. The method of claim 1, wherein the first nucleic acid encodes a first portion of the Cas9 protein having a first split-intein and wherein the second nucleic acid encodes a second portion of the Cas9 protein having a second split-intein complementary to the first split-intein and wherein the first portion of the Cas9 protein and the second portion of the Cas9 protein are joined together to form the Cas9 protein.

10. (canceled)

11. The method of claim 1, wherein the first portion of the Cas9 protein is the N-terminal lobe of the Cas9 protein and the second portion of the Cas9 protein is the C-terminal lobe of the Cas9 protein.

12.-15. (canceled)

16. The method of claim 1, wherein the Cas9 protein is an enzymatically active Cas9 protein or a Cas9 protein nickase.

17. A method of altering a target nucleic acid in a cell of a subject comprising
delivering to the cell of the subject a first nucleic acid encoding a first portion of a Cas9 protein and a guide RNA (gRNA) wherein the first nucleic acid is within a first vector,
delivering to the cell of the subject a second nucleic acid encoding a second portion of the Cas9 protein and optionally a transcriptional regulator wherein the second nucleic acid is within a second vector,
wherein the cell expresses the first portion of the Cas9 protein, the gRNA and the second portion of the Cas9 protein or the second portion of the Cas9 and the transcriptional regulator fusion protein,
wherein the first portion of the Cas9 protein and the second portion of the Cas9 protein, or the first portion of the Cas9 protein and the second portion of the Cas9 and the transcriptional regulator fusion protein are joined together to form the Cas9 protein or the Cas9 fusion protein, and
wherein the gRNA and the Cas9 protein, or the gRNA and the Cas9 fusion protein form a co-localization complex with the target nucleic acid and alter the expression of the target nucleic acid.

18.-34. (canceled)

35. A method of modulating a target gene expression in a cell comprising providing to the cell a first recombinant adeno-associated virus comprising a first nucleic acid encoding an N-terminal portion of the Cas9 protein (Cas9^{N}) and a gRNA,
providing to the cell a second recombinant adeno-associated virus comprising a second nucleic acid encoding a fusion protein comprising a C-terminal portion of the Cas9 protein (Cas9^{C}) fused with a transcriptional regulator (TR),
wherein the cell expresses the Cas9^{N} protein and the Cas9^{C}-TR fusion protein and joins them to form a full length Cas9^{FL}-TR fusion protein, and
wherein the cell expresses the gRNA, and the gRNA directs the Cas9^{FL}-TR fusion protein to the target gene and modulates target gene expression.

36. The method of claim 35, wherein the Cas9 is a Type II CRISPR system Cas9 and the transcriptional regulator is VPR.

37. The method of claim 35, wherein the first nucleic acid encodes the N-terminal portion of the Cas9 protein (Cas9^{N}) having a N-split-intein RmaIntN and wherein the second nucleic acid encodes the fusion protein comprising a C-terminal portion of the Cas9 protein (Cas9^{C}) fused with a transcriptional regulator (TR) and having a C-split-intein RmaIntC and wherein the first portion of the Cas9 protein and the second portion of the Cas9 protein are joined together to form the Cas9 protein.

38.-42. (canceled)

43. The method of claim 35, wherein the N-terminal portion of the Cas9 protein (Cas9^{N}) is the N-terminal lobe of the Cas9 protein up to amino acid V713 and the C-terminal portion of the Cas9 protein is the C-terminal lobe of the Cas9 protein beginning at D714.

44. (canceled)

45. The method of claim 35, wherein the gRNA has truncated spacer sequence and directs Cas9^{FL}-TR fusion protein binding to target DNA without cleaving the target DNA.

46. A method of imaging a target nucleic acid in a cell comprising
providing to the cell a first recombinant adeno-associated virus comprising a first nucleic acid encoding an N-terminal portion of the Cas9 protein (Cas9^{N}) and a gRNA,
providing to the cell a second recombinant adeno-associated virus comprising a second nucleic acid encoding a fusion protein comprising a C-terminal portion of the Cas9 protein (Cas9^{C}) fused with a fluorescent protein,
wherein the cell expresses the Cas9^{N} protein and the Cas9^{C} fluorescent fusion protein and joins them to form a full length Cas9^{FL} fluorescent fusion protein, and
wherein the cell expresses the gRNA, and the gRNA directs the Cas9^{FL} fluorescent fusion protein to the target nucleic acid and produces fluorescent imaging of the target nucleic acid.

47.-75. (canceled)


SEQUENCE LISTING (50 sequences)

SEQ ID NO: 1 - protein, 14 aa, Artificial Sequence
  Description of Artificial Sequence Synthetic peptide
CASSLDRGQDTQYF

SEQ ID NO: 2 - protein, 1368 aa, Streptococcus pyogenes [sequence omitted]

SEQ ID NO: 3 - DNA, 83 bp, Artificial Sequence
  Description of Artificial Sequence Synthetic oligonucleotide
gttttagagctagaaatagcaagttaaaataaggctagtccgttatcaacttgaaaaagtggcaccgagtcggtgcttttttt

SEQ ID NO: 4 - protein, 9 aa, Streptococcus pyogenes
ITGLYETRI

SEQ ID NO: 5 - protein, 12 aa, Streptococcus pyogenes
IVDEVAYHEKYP

SEQ ID NO: 6 - protein, 10 aa, Streptococcus pyogenes
WDPKKYGGFD

SEQ ID NO: 7 - protein, 19 aa, Adeno-associated virus
FMIPQYGYLTLNDGSQAVG

SEQ ID NO: 8 - protein, 10 aa, Adeno-associated virus
MNPLIDQYLY

SEQ ID NO: 9 - protein, 12 aa, Adeno-associated virus
TQNNNSEFAWPG

SEQ ID NO: 10 - DNA, 40 bp, Artificial Sequence
  Description of Artificial Sequence Synthetic oligonucleotide
gggccatgtggacatccatgaggtgagacagtgccagcgt

SEQ ID NO: 11 - DNA, 38 bp, Artificial Sequence
  Description of Artificial Sequence Synthetic oligonucleotide
ggcctgaagccactacagctgctggagatcaaggctcg

SEQ ID NO: 12 - DNA, 27 bp, Artificial Sequence
  Description of Artificial Sequence Synthetic oligonucleotide
ggccctagcatctaagttctcgcaggc

SEQ ID NO: 13 - DNA, 35 bp, Artificial Sequence
  Description of Artificial Sequence Synthetic oligonucleotide
ggtcattccatctcagctgtgacagcagcgcagaa

SEQ ID NO: 14 - DNA, 30 bp, Artificial Sequence
  Description of Artificial Sequence Synthetic oligonucleotide
gtcaagcccaaagtctctccgggacctctt

SEQ ID NO: 15 - DNA, 29 bp, Artificial Sequence
  Description of Artificial Sequence Synthetic oligonucleotide
ggaatcccggtgctgccgctaccccctca

SEQ ID NO: 16 - DNA, 21 bp, Artificial Sequence
  Description of Artificial Sequence Synthetic oligonucleotide
gctagagaataggaacttctt

SEQ ID NO: 17 - DNA, 21 bp, Artificial Sequence
  Description of Artificial Sequence Synthetic oligonucleotide
gaaagaattgatttgataccg

SEQ ID NO: 18 - DNA, 19 bp, Artificial Sequence
  Description of Artificial Sequence Synthetic oligonucleotide
gatccccatcaagctgatc

SEQ ID NO: 19 - DNA, 21 bp, Artificial Sequence
  Description of Artificial Sequence Synthetic oligonucleotide
ggtatgctatacgaagttatt

SEQ ID NO: 20 - DNA, 15 bp, Artificial Sequence
  Description of Artificial Sequence Synthetic oligonucleotide
gctcgagataagacc

SEQ ID NO: 21 - DNA, 15 bp, Artificial Sequence
  Description of Artificial Sequence Synthetic oligonucleotide
gctaaagtcatccgc

SEQ ID NO: 22 - DNA, 15 bp, Artificial Sequence
  Description of Artificial Sequence Synthetic oligonucleotide
ggttcttatttgcgt

SEQ ID NO: 23 - DNA, 15 bp, Artificial Sequence
  Description of Artificial Sequence Synthetic oligonucleotide
ggaaatcaaagcggc

SEQ ID NO: 24 - DNA, 15 bp, Artificial Sequence
  Description of Artificial Sequence Synthetic oligonucleotide
gaaggagttcctcgg

SEQ ID NO: 25 - DNA, 15 bp, Artificial Sequence
  Description of Artificial Sequence Synthetic oligonucleotide
gaggaggtccacttc

SEQ ID NO: 26 - DNA, 54 bp, Artificial Sequence
  Description of Artificial Sequence Synthetic primer
  modified_base (29)..(34): a, c, t, g, unknown or other
ctttccctacacgacgctcttccgatctnnnnnnctggagtgttagagtgggcg

SEQ ID NO: 27 - DNA, 48 bp, Artificial Sequence
  Description of Artificial Sequence Synthetic primer
ggagttcagacgtgtgctcttccgatctgactgccccatggaaagaca

SEQ ID NO: 28 - DNA, 58 bp, Artificial Sequence
  Description of Artificial Sequence Synthetic primer
  modified_base (29)..(34): a, c, t, g, unknown or other
ctttccctacacgacgctcttccgatctnnnnnngggccatgaaaggaaaaatgaagt

SEQ ID NO: 29 - DNA, 48 bp, Artificial Sequence
  Description of Artificial Sequence Synthetic primer
ggagttcagacgtgtgctcttccgatctgcctctggggtttgcttggt

SEQ ID NO: 30 - DNA, 67 bp, Artificial Sequence
  Description of Artificial Sequence Synthetic primer
  modified_base (29)..(34): a, c, t, g, unknown or other
ctttccctacacgacgctcttccgatctnnnnnngagatataagctgaataaggccaatgacatact

SEQ ID NO: 31 - DNA, 48 bp, Artificial Sequence
  Description of Artificial Sequence Synthetic primer
ggagttcagacgtgtgctcttccgatctctactgctctttcctgccga

SEQ ID NO: 32 - DNA, 21 bp, Artificial Sequence
  Description of Artificial Sequence Synthetic primer
gcctactcgctgctgcccatt

SEQ ID NO: 33 - DNA, 21 bp, Artificial Sequence
  Description of Artificial Sequence Synthetic primer
cctggagacccccaaaagctc

SEQ ID NO: 34 - DNA, 19 bp, Artificial Sequence
  Description of Artificial Sequence Synthetic probe
agatcttcccacttcaggt

SEQ ID NO: 35 - DNA, 21 bp, Artificial Sequence
  Description of Artificial Sequence Synthetic primer
ggaacccctagtgatggagtt

SEQ ID NO: 36 - DNA, 16 bp, Artificial Sequence
  Description of Artificial Sequence Synthetic primer
cggcctcagtgagcga

SEQ ID NO: 37 - DNA, 21 bp, Artificial Sequence
  Description of Artificial Sequence Synthetic probe
cactccctctctgcgcgctcg

SEQ ID NO: 38 - protein, 831 aa, Artificial Sequence
  Description of Artificial Sequence Synthetic polypeptide
MAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVCLAGDTLITLADGRRVPIRELVSQQNFSVWALNPQTYRLERARVSRAFCTGIKPVYRLTTRLGRSIRATANHRFLTPQGWKRVDELQPGDYLALPRRIPTAS

SEQ ID NO: 39 - DNA, 2496 bp, Artificial Sequence
  Description of Artificial Sequence Synthetic polynucleotide
atggccccaaagaagaagcggaaggtcggtatccacggagtcccagcagccgacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttgtctggctggcgatactctcattaccctggccgatggacgacgagtgcctattagagaactggtgtcacagcagaatttttccgtgtgggctctgaatcctcagacttaccgcctggagagggctagagtgagtagagctttctgtaccggcatcaaacctgtgtaccgcctcaccactagactggggagatccattagggccactgccaaccaccgatttctcacacctcagggctggaaacgagtcgatgaactccagcctggagattacctggctctgcctaggagaatccctactgcctcctga

SEQ ID NO: 40 - protein, 970 aa, Artificial Sequence
  Description of Artificial Sequence Synthetic polypeptide
MAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVSRAGSGATNFSLLKQAGDVEENPGPMPAMKIECRITGTLNGVEFELVGGGEGTPEQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARSRAR

SEQ ID NO: 41 - DNA, 2913 bp, Artificial Sequence
  Description of Artificial Sequence Synthetic polynucleotide
atggcggcggcgtgcccggaactgcgtcagctggcgcagagcgatgtgtattgggatccgattgtgagcattgaaccggatggcgtggaagaagtgtttgatctgaccgtgccgggcccgcataactttgtggcgaacgatattattgcgcataactctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtctcgagctggatccggagccacgaacttctctctgttaaagcaagcaggggacgtggaagaaaaccccggtcctatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagctcgatga

SEQ ID NO: 42 - protein, 1271 aa, Artificial Sequence
  Description of Artificial Sequence Synthetic polypeptide
MAAACPELRQLAQSDVYWDPIVSIEPDGVEEVFDLTVPGPHNFVANDIIAHNSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSRADPKKKRKVSPGIRRLDALISTSLYKKAGYKEASGSGRADALDDFDLDMLGSDALDDFDLDMLGSDALDDFDLDMLGSDALDDFDLDMLINSRSSGSPKKKRKVGSQYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALLGSGSGSRDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 43 - DNA, 3816 bp, Artificial Sequence
  Description of Artificial Sequence Synthetic polynucleotide
atggcggcggcgtgcccggaactgcgtcagctggcgcagagcgatgtgtattgggatccgattgtgagcattgaaccggatggcgtggaagaagtgtttgatctgaccgtgccgggcccgcataactttgtggcgaacgatattattgcgcataactctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttga

SEQ ID NO: 44 - DNA, 23 bp, Mus sp.
aagtctctccgggacctcttggg

SEQ ID NO: 45 - DNA, 23 bp, Mus sp.
aaggctctccaggacctcttggg

SEQ ID NO: 46 - DNA, 23 bp, Mus sp.
aagtatctctgggacctcttcag

SEQ ID NO: 47 - DNA, 23 bp, Mus sp.
gagtccctccgggagctcttggg

SEQ ID NO: 48 - DNA, 23 bp, Mus sp.
gtgctgccgctaccccctcacgg

SEQ ID NO: 49 - DNA, 23 bp, Mus sp.
gtgctgcagctgccacctcaggg

SEQ ID NO: 50 - DNA, 23 bp, Mus sp.
gtgcagccgctaccaccacaaag